(19) United States
(12) Patent Application Publication
(10) Pub. No.: US 2023/0034402 A1
(43) Pub. Date: Feb. 2, 2023

(54) ATTACHMENT OF BIOLOGICAL AND NON-BIOLOGICAL OBJECTS

(71) Applicant: RESISTELL AG, Muttenz (CH)

(72) Inventors: Michael SIEGERT, Leeuwarden (NL); Eric DELARZE, Muttenz (CH); Amanda LURASCHI, Muttenz (CH); Michal SWIATKOWSKI, Muttenz (CH); Grzegorz WIELGOSZEWSKI, Muttenz (CH)

(73) Assignee: RESISTELL AG, Muttenz (CH)

(21) Appl. No.: 17/788,572

(22) PCT Filed: Dec. 23, 2020
(86) PCT No.: PCT/EP2020/087821

§ 371 Date: Jun. 23, 2022

(30) Foreign Application Priority Data
Dec. 23, 2019 (EP) PCT/EP2019/086974

(51) Int. Cl. G01N 33/543

(52) U.S. Cl. CPC G01N 33/54393

(57) ABSTRACT

A kit-of-parts for attaching an object on a substrate (1, 1′) having (i) at least a first solution having at least one first compound, wherein the first compound is at least one of a gelling agent, a gellable agent, and a thickening agent, and (ii) at least a first substrate (1, 1′) having a surface (2, 2′). The first solution is suitable for forming at least a first dispersion of an object in the first solution when a the object is added to the first solution. The first dispersion is suitable for attaching the object on a functionalized surface (3, 3′) of the substrate (1, 1′) when the first dispersion is added to the functionalized surface (3, 3′) of the substrate (1, 1′).

### TECHNICAL FIELD

The present invention relates to a kit-of-parts for attaching a preferably biological object and/or a non-biological object on a substrate according to claim 1, a method of producing a kit-of-parts according to claim 20, the use of a substrate for attaching a preferably biological object and/or a non-biological object according to claim 22, the use of a first solution for attaching a preferably biological object and/or a non-biological object on a functionalized substrate according to claim 28, a method of attaching a preferably biological object and/or a non-biological object on a substrate according to claim 34, the use of a kit-of-parts in a method of attaching a preferably biological object and/or non-biological object according to claim 43, and the substrate comprising a preferably biological object and/or non biological object being attached thereon according to claim 44, respectively.

### PRIOR ART

Attachment of biological matter to abiotic surface is of general interest in many industries. It is a natural, yet often an undesired phenomenon, for example in the case of biofilms which can lead to microbial influenced corrosion, infestation of medical devices, clogging (e.g. pipes, bioreactors, or wastewater plants), and so forth. Likewise, the attachment of biological matter can also be intended, for example in medical implants, biosensors, fuel cells, imaging, cell cultures, bioreactors, and numerous other research and industry applications.

One very recent application for the attachment of biological matter is the measurement of nanomotion as disclosed in EP 2 766 722 B1. Namely, EP 2 766 722 B1 discloses a rapid antibiotic susceptibility test (AST) that is based on atomic force microscopy. The AST is called nanomotion AST because it uses movements of AFM cantilevers that are caused by microorganisms and other biological material attached to these cantilevers. The nanomotion AST provides the same results within a few minutes to hours regardless of strain identity. It comprises a device and a cantilever mounted in the device. Once an effective toxin, for example an antibiotic, is added, cells attached to the cantilevers die and the movements of the cantilevers' cease. Obviously, this technology requires robust cell attachment.

A major issue with nanomotion ASTs is cell attachment to cantilevers. It is not unusual that only one out of five attempts to attach cells to cantilevers is successful. Other bioactivity tests require cell attachment as well. For example, cells can be attached on a surface to detect their movements using microscopy. In other cases, cellular vital signs are not necessarily of interest, for example in the case of cell attachment aided by polymers on Matrix-assisted laser desorption ionization time-of-flight mass spectrometry (MALDI-TOF MS) trays. However, the use of such polymers as additives leads to cell aggregation and cell death which is often not desired, for example when vital signs are to be recorded. Some negative consequences of cell aggregation are that a clean distinction of single cells is impossible, that cell aggregates change their metabolism and that biofilms form, or that cellular signals become unevenly distributed. In summary, the applications for bio-immobilization are plentiful and there is a need for rapid and robust cell attachment to abiotic surfaces, avoiding cell death or aggregation.

### SUMMARY OF THE INVENTION

It is therefore an object of the present invention to enable an improved attachment of a biological object on a substrate. In particular, it is an object to enable an attachment of a biological object on a substrate in a rapid and robust manner.

This object is achieved with a kit-of-parts according to claim 1, with a method of producing a kit-of-parts according to claim 20, with the use of a substrate for attaching an object according to claim 22, with the use of a first solution for attaching an object on a functionalized substrate according to claim 28, with a method of attaching an object on a substrate according to claim 34, the use of a kit-of-parts in a method of attaching an object according to claim 43, and with a substrate comprising an object being attached thereon according to claim 44, respectively.

In particular, a kit-of-parts for attaching a biological object on a substrate is provided, which comprises (i) at least a first solution comprising at least one first compound, wherein the first compound is at least one of a gelling agent, a gellable agent, and a thickening agent, and (ii) at least a first substrate comprising a surface. The first solution is suitable for forming at least a first dispersion of at least one biological object in the first solution when at least one biological object is added to the first solution. The first dispersion is suitable for attaching the biological object on the surface of the substrate when the first dispersion is added to the surface of the substrate.

The kit-of-parts preferably further comprises instructions for the attachment of the object, wherein the instructions comprise the step of preparing the first dispersion by dispersing the at least one object in the first solution. Optionally, the instructions may further comprise the step of adding the first dispersion to the optionally functionalized surface of the substrate so as to attach the object on the optionally functionalized surface of the substrate.

To this end it is conceivable that at least a first part of the surface of the substrate is functionalized, see further below. In this case, the first dispersion and the functionalized part of the substrate are preferably suitable for attaching the biological object on the functionalized surface of the substrate when the first dispersion is added to the functionalized surface of the substrate.

Since the first solution is used for forming a dispersion comprising the potentially living objects to be attached it is preferred that the first solution comprises a pH-value that is physiological. In particular, it is preferred that the pH-value of the first solution is in the range of about 6 to 8, particularly preferably about 7. It is furthermore preferred that a temperature of the first solution is and/or remains below a temperature being lethal for the objects to be attached. As such, it is conceivable that the instructions of the kit-of-parts furthermore comprise the step of i) preparing the first dispersion at a temperature being at or below about 37° C. and/or in the range of between about 15° C. to 40° C., more preferably in the range of about 20° C. to 25° C. and/or ii) adding the first dispersion to the optionally functionalized surface of the substrate at a temperature being at or below about 37° C. and/or in the range of about 15° C. to 40° C., more preferably in the range of about 20° C. to 25° C. The instructions may further comprise the step of heating the first solution to a temperature of at least 40° C. or more, for example of at least 60° C. or more such as 90° C. or more so as to dissolve the first compound. Consequently, the instructions may further comprise the step of cooling the heated first solution to a temperature of 37° C. or less before the one or more objects are added to the first solution.

Likewise, a method of producing a kit-of-parts for attaching a biological object on a substrate is provided, the method comprising the steps of (i) providing at least a first solution comprising at least one first compound, wherein the first compound is at least one of a gelling agent, a gellable agent, and a thickening agent, and (ii) providing at least a first substrate comprising a surface. The first solution is suitable for forming at least a first dispersion of at least one biological object in the first solution when at least one biological object is added to the first solution. The first dispersion is suitable for attaching the biological object on the surface of the substrate when the first dispersion is added to the surface of the substrate.

Also, in this case it is conceivable that at least a first part of the surface of the substrate is functionalized, wherein the first dispersion and the functionalized part of the substrate are preferably suitable for attaching the biological object on the functionalized surface of the substrate when the first dispersion is added to the functionalized surface of the substrate.

Furthermore, the present invention also relates to the use of substrate for attaching at least one biological object dispersed in at least a first solution, wherein the first solution comprises at least one first compound, wherein the first compound is at least one of a gelling agent, a gellable agent, and a thickening agent, and wherein at least a first part of the surface of the substrate is preferably functionalized.

In addition, the present invention also relates to the use of a first solution for attaching at least one biological object dispersed in the first solution on a surface of a substrate, wherein the first solution comprises at least one first compound, wherein the first compound is at least one of a gelling agent, a gellable agent, and a thickening agent, and wherein at least a first part of the surface of the substrate is preferably functionalized.

Moreover, a method of attaching a biological object on a substrate is provided, which method comprises the steps of (i) preparing at least a first dispersion of at least one biological object in a first solution, and (ii) adding the first dispersion to the surface of the substrate, whereby the biological object is attached on the surface of the substrate. The first solution comprises at least one first compound, wherein the first compound is at least one of a gelling agent, a gellable agent, and a thickening agent.

Furthermore, the present invention relates to the use of a kit-of-parts as described above and further below in a method of attaching an object on a surface of a substrate as described above and further below.

The present invention also relates to a substrate comprising at least one object being attached thereon, wherein the substrate comprises at least one surface and at least one first layer being arranged on at least a part of the surface and being formed from at least a first dispersion as obtained in the method of attaching an object on a surface of a substrate as described above and further below.

For the sake of completeness, it is mentioned again that it is conceivable that at least a first part of the surface of the substrate is functionalized, wherein the first dispersion and the functionalized part of the substrate are preferably suitable for attaching the biological object on the functionalized surface of the substrate when the first dispersion is added to the functionalized surface of the substrate.

The inventors have surprisingly found out that the use of a first solution comprising at least one of a gelling agent, gellable agent, and a thickening agent possibly in combination with a functionalized surface of the substrate results in an improved attachment of the biological object as compared to the attachment methods known in the state of the art. In particular, the invention enables an attachment of the biological objects in high numbers. Furthermore, the formation of an agglomerations of the attached objects is reduced or even prevented. In other words, a more homogeneous attachment is achieved. At the same time, it also enables a rapid and facile attachment that can be carried out using standard laboratory equipment. Besides, and as opposed to substrates known in the art which have to be used within a few hours, the first solution comprising the at least one first compound and the preferably functionalized substrate can be stored over several weeks. To this end it is therefore preferred to provide the first solution comprising the first compound in a storage means such as a container or the like.

The object preferably is a biological object and/or a non-biological object. That is, it is conceivable to attach one or more biological objects, or one or more non-biological objects, or a mixture of one or more biological objects as well as one or more non-biological objects. The biological object preferably is at least one of a cell, a virus such as a phage, and a matter of biological origin such as peptides, proteins, polysaccharides, vesicles, protein-RNA co-polymers, protein-DNA co-polymers, capsules, spores, and so forth. The matter of biological origin is preferably particulate. The cell can correspond to pathogenic or non-pathogenic prokaryotic cells, eukaryotic cells, aggregates thereof, or tissue. Pathogenicity may occur in humans, animals, plants, or fungi. The cells can have different genotypic and phenotypic traits and do not need to be of the same identity. However, it is important to note that further biological objects that have not been explicitly mentioned here can be likewise attached. However, it is also conceivable that the object is a non-biological object such as a protein, lipid, nucleic acid such as DNA, a nanotube or nano bead made of elementary carbon metal oxides such as titanium oxide, a nanodevice, a glucide, a hydrocarbon, an aliphatic or aromatic polymer such as a phenolic polymer, and the like.

In the following, further aspects of the kit-of-parts, the method of producing a kit-of-parts, the use of the substrate for attaching the biological object and/or the non-biological object, the use of the first solution for attaching the biological object and/or the non-biological object on the substrate, the method of attaching the biological object and/or the non-biological object on the substrate, the use of a kit-of-parts in a method of attaching an object on a surface of a substrate, as well as the substrate comprising at least one object being attached thereon are discussed. For convenience, no explicit distinction is made between the kits-of-parts, the methods, the uses, and the substrate. Instead, any explanations provided below likewise apply to all of them.

As already mentioned, it is conceivable that at least a first part of the surface of the substrate is functionalized, and wherein the first dispersion and the functionalized surface of the substrate are suitable for attaching the object on the functionalized surface of the substrate when the first dispersion is added to the functionalized surface of the substrate. To this end, pre-treated substrates having a functionalized surface can be provided. However, it is likewise conceivable to provide untreated, i.e. non-functionalized substrates together with means for functionalizing said substrates. In the former case the kit-of-parts could further comprise the pre-treated substrates. In the latter case the kit-of-parts could further comprise the untreated substrates as well as the means for functionalizing said untreated substrates. In the latter case it is conceivable that the instructions furthermore comprise the step of adding the second solution comprising the second compound to the surface of the substrate so as to functionalize the surface of the substrate. This step of adding the second solution to the substrate is particularly preferably performed prior to the step of adding the first dispersion to the substrate.

It should be noted that only one part of the surface of the substrate can be functionalized, or that two or more parts of the surface of the substrate can be functionalized. Said two or more functionalized parts could be arranged immediately adjacent to one another or at a distance from one another. However, it is likewise conceivable that the entire surface of the substrate is functionalized. Explanations provided herein with reference to a partial functionalization of the surface of the substrate likewise apply to an entire functionalization of the surface of the substrate.

That is to say, it is conceivable that at least one of i) at least a first part of the surface of the substrate is preferably physically and/or chemically functionalized and ii) the kit-of-parts further comprises at least one second compound being suitable for chemically functionalizing at least a first part of the surface of the substrate.

Hence, the functionalization of the surface of the substrate can correspond to a chemical functionalization that is achieved by applying at least one second compound to the surface of the substrate, wherein the second compound interacts with the surface of the substrate. Said interaction between the second compound and the surface of the substrate results in the functionalization of the surface of the substrate. In other words, the surface of the substrate is functionalized by its interaction with the second compound. To this end it is therefore conceivable to provide at least a second compound in the kits-of-parts. This is particularly preferred when the second compound is directly applicable onto the surface of the substrate. However, the second compound can also be provided in a solution. Hence, the kit-of-parts could further comprise at least a second solution comprising the at least one second compound. Said second solution is preferably provided in a suitable storage means such as a container or the like. Hence, during the method of attaching the biological object and/or the non-biological object on the surface of the substrate it is conceivable to prepare at least one second solution and to add that second solution to the substrate so as to functionalize the surface of the substrate in a first step, and to then add the first dispersion comprising the first solution and the biological objects and/or the non-biological objects to the functionalized surface of the substrate in a second step. However, it is likewise conceivable to provide the kit-of-parts with a pretreated substrate, wherein its surface has been functionalized as just described before the functionalized substrate is arranged in the kits-of-parts. In this case a user can simply prepare the first dispersion and can readily add said first dispersion to the functionalized substrate.

Additionally, or alternatively, the functionalization of the surface of the substrate can correspond to a physical functionalization that is achieved by generating a surface structure in the surface of the substrate and/or by generating at least one layer on the surface of the substrate.

The generation of a surface structure in the surface of the substrate is preferably performed by surface modification methods that are well-known in the art. Said surface modification methods can correspond to chemical or physical surface modification methods and include, without limitation: etching with bases (e.g. KOH), etching with acids (e.g. HF), ion milling, deep reactive ion etching (DRIE), focused ion beam (FIB), scanning electron microscopy (SEM), as high energy electrons can modify surfaces, ion implantation/doping, electroplating, epitaxy methods such as liquid phase epitaxy (LPE), molecular beam epitaxy (MBE), VPE—vapour phase epitaxy (VPE), metal-organic vapor phase epitaxy (MOVPE), sputtering methods such as thin film sputtering methods, DC-sputtering, RF-sputtering, magnetron sputtering, as well as mechanical modifications such as scratching or laser-based modifications.

The surface structure can be seen as a surface relief that is generated in the surface of the substrate. That is, it is preferably constituted by several elevations and recesses. The dimensions of the surface structure, i.e. a height or depth of said elevations and recesses, preferably are microscopic dimensions. In other words, the dimensions of the surface structure preferably essentially correspond to the size of the object that shall be attached.

The one or more layers that are generated on the surface of the substrate are preferably a monolayer, particularly preferably an atomic monolayer. A preferred thickness of the one or more layers lies in the range of several hundred nanometers.

The at least one layer can comprise at least one metal compound and/or at least one oxide compound and/or at least one silicon compound and/or at least one nitride compound and/or at least one sulphide compound. The metal compound preferably comprises or consists of a noble metal such as gold, platinum, and palladium and combinations thereof. The oxide compound is preferably selected from titanium oxide, iron oxide, nickel oxide, aluminium oxide, silicone dioxide, cupric oxide, cuprous oxide and combinations thereof, such as an iron-nickel-oxide. The nitride compound preferably corresponds to silicon nitride. The sulphide compound is preferably selected from molybdenum sulphide, iron sulphide, nickel sulphide, iron-nickel sulphide, manganese sulphide, copper sulphide, titanium sulphide, uranium sulphide, cobalt sulphide, aluminium sulphide, chromium sulphide, yttrium sulphide and combinations thereof.

The second compound preferably is at least one of a polymer or a copolymer thereof, a polymerizable agent, a cross-linking agent, and a compound comprising at least one functional group. The polymer or the copolymer thereof and/or the polymerizable agent can be at least one of a polysaccharide compound, a polyaminosaccharide compound, a polyaminoacid compound, a polydopamine compound, a glycoproteine compound, a nucleic acid compound, an epoxy resin compound, a polysilane compound, a polysiloxane compound, a polyphosphate compound, a boron nitride polymer compound, a fluoropolymer compound, a polyallylamine compound, a polysulphide compound, a polyphenol compound, and a silicon-based polymer. The polyaminosaccharide compound preferably is chitosan. Additionally or alternatively the polyaminoacid compound preferably is polylysine, particularly preferably poly-D-lysine. Additionally or alternatively the glycoprotein compound preferably is laminin. Additionally or alternatively the nucleic acid compound preferably is desoxy ribonucleic acid. Additionally or alternatively the epoxy resin compound preferably is at least one of a bisphenol polymer compound and polyacetylene compound. Additionally or alternatively the polyphenol compound preferably is a polyphenolic protein, preferably a polyphenolic protein secreted by Mytilus sp., such as the polyphenolic protein secreted by Mytilus edulis. 110-140 kDa that is commercially available as Cell-Tak™. Additionally or alternatively, the second compound can be a recombinant Mytilus protein, preferably a recombinant Mytilus protein being produced by bacteria such as MAPTRix™, 23 kDa. Additionally or alternatively, the silicon-based polymer preferably corresponds to a polymeric organosilicon compound, preferably polydimethylsiloxane (PDMS). If polydimethylsiloxane is used as the second compound it is particularly preferred to additionally provide one or more curing agents that are configured to cure the said second compound. Additionally or alternatively the polyallylamine compound preferably comprises primary and/or secondary and/or tertiary polymers and preferably corresponds to a copolymer of polyallylamine and polystyrene. A conceivable copolymer of polyallylamine and polystyrene corresponds to the commercially available compound Bacpro® II. The cross-linking agent can be at least one of a homobifunctional cross-linking agent, a heterobifunctional cross-linking agent, and a photoreactive cross-linking agent, preferably an aldehyde-comprising cross-linking agent, particularly preferably glutaraldehyde. A homobifunctional cross-linking agent is understood as an agent comprising identical reactive groups at either ends. A heterobifunctional cross-linking agents is understood as an agent that possesses two different reactive groups. A photoreactive cross-linking agent is understood as a heterobifunctional cross-linking agent that become reactive upon exposure to radiation. The functional group can be at least one of an organic group, an inorganic group, and an organometallic group, preferably an organosilicon compound or an organosulfur compound, particularly preferably (3-aminopropyl)triethoxysilane (APTES) or 4-aminothiophenol (4-ATP).

The second solution preferably comprises at least one of a protic solvent, an aprotic solvent, a nonpolar solvent, a polar solvent, an organic compound, an inorganic compound, a liquid gas, and a melt. For example, the second solution could comprise acetone, ethanol, ethylene glycol, toluene, or naphthalene. It is preferred that the second solution is an aqueous solution, particularly preferably an aqueous solution that further comprises at least one of a polar water-soluble solvent such as an alcohol, a dissolved salt such as sodium chloride, and an acid such as acetic acid or hydrochloric acid. Depending on the chemical or physical properties of these compounds, it is preferred to apply the second solution to the substrate at an elevated temperature and/or pressure, see below.

The first compound can be at least one of a polymer and a polymerizable agent. Preferably the first compound is at least one of a polysaccharide, an amide-based polymer, a silicon-based polymer, and an ionomer. The polysaccharide is preferably selected from agarose, agar, alginate, dextran. Additionally or alternatively the amide-based polymer preferably corresponds to polyacrylamide. Additionally or alternatively the silicon-based polymer preferably corresponds to a polymeric organosilicon compound, preferably polydimethylsiloxane. Additionally or alternatively, the ionomer preferably corresponds to an inorganic polymer, preferably to a fluorinated polymer. The ionomer particularly preferably corresponds to the commercially available compound known as Nafion®.

In other words, the first compound may be chosen among saccharides, disachharides, oligosacchraides, or polysachharides and their respective mixtures. Suitable monosaccharides are in particular glucose, fructose and galactose. Suitable disaccharides are lactose, sucrose and maltose. Suitable polysaccharides are agarose, galactan, agaropectin, alginate, and mixtures thereof. An example of such a mixture of polysaccharides is agar. The first compound may further be chosen among synthetic polymers such as polyacrylamide, polyalkylene glycols, polysiloxanes or fluorpolymers. Suitable polyalkylene glycols may be chosen among polyethylene glycols, polypropylene glycols or copolymers thereof. Suitable polysiloxanes may be chosen among polydimethylsiloxane. Suitable fluoropolymers may be chosen among polymers or copolymers of tetrafluoroethylene such as polytetrafluoroethylene (PTFE) or sulphonated tetrafluoroethylene (Nafion®).

The second compound may be chosen among aldehydes, dialdehydes or polyaldehydes and their respective mixtures. Suitable dialdehydes are aliphatic dialdehydes or aromatic dialdehydes. An example of an aliphatic dialdehyde is glutaraldehyde. The second compound can be further chosen among polyelectrolytes such as poly(sodium-p-styrene sulfonate), poly(allylamine hydrochloride) or copolymers thereof, polynucleotides, polypeptides, polysaccharides such as a polyaminosaccharide for example polyglucosamin, also known as chitosan, polypeptides such as poly-alpha-lysine or poly-D-lysine, proteins such as collagens, glycoproteins such as laminins or mussel adhesive proteins, enzymes, or aminosilanes such as APTES (3-aminopropyl)-triethoxysilane, APDEMS (3-aminopropyl)-diethoxy-methylsilane, APDMES (3-aminopropyl)-dimethyl-ethoxysilane, or APTMS (3-aminopropyl)-trimethoxysilane. The second compound may be further chosen among polystyrenes or polyallylamines and mixtures thereof. Examples of polystyrenes are polymers of sodium-styrene sulfonate, such as poly(sodium-p-styrene sulfonate. A suitable mixture of polystyrenes and polyallylamines is Poly(sodium-p-styrene sulfonate)/poly(allylamino hydrochloride), also known as PSS/PAH, i.e. a polyelectrolyte. The PAH-PSS co-polymer is a so-called layer-by-layer polymer, wherein one layer is formed by PAH (poly(allylamino hydrochloride)) and the other layer is formed by PSS (Poly(sodium-p-styrene sulfonate)). PAH is charged positively and PSS is charged negatively, which makes the PAH-PSS co-polymer advantageous for attaching positively or negatively charged objects. The layer being proximate to the object to be attached is chosen in accordance with the charge of the object to be attached. An example of a mussel adhesive protein is the Mussel Adhesion Protein extracellular matrix (MAPTrix™). The Mussel Adhesion Protein extracellular matrix (MAPTrix™) as used herein has been commercially bought as the MAPTrix™ Adhesive Kit from the supplier Sigma-Aldrich, which indicates Kollodis Biosciences as producer. It corresponds to a Tyrosinase-pretreated powder with a molecular weight of about 23 kDa. The MAPTrix™ Adhesive Kit is a formulation of polyphenolic mussel adhesive proteins recombinantly produced in Kollodi's proprietary E. coli expression system. The recombinant mussel adhesive protein is a hybrid of Mytilus edulis fp-1 and fp-5 or a hybrid of Mytilus edulis fp-1, fp-3 and fp-5. The second compound may be further chosen among thiols such as aromatic thiols. An example of an aromatic thiol is tiophenol. A suitable tiophenol is aminothiophenol such as 2-aminothiopnelol, 3-aminothiopnelol, or 4-aminothiopnelol. As mentioned earlier, the object preferably is a biological object. Suitable biological objects are cells. The cells may be prokaryotic cells and/or eukaryotic cells. Examples of prokaryotic cells are bacteria such as enterobacteria and mycobacteria. Examples of eukaryotic cells are mammalian cells and yeast. An example of enterobacteria is Escherichia coli. An example of mycobacteria is Mycobacterium smegmatis. An example of mammalian cells are Vero cells. An example of yeast is Candida albicans.

Preferred first compounds used for forming the first dispersion and preferred second compounds to which the first dispersion is added in order to attach the object to the substrate are the following.

For example, it is preferred to use agarose as the first compound, an Enterobacterium such as Escherichia coli as the object to be attached, and poly-D-lysine as the second compound.

It is also preferred to use agar as the first compound, an Enterobacterium such as Escherichia coli as the object to be attached, and poly-D-lysine as the second compound.

It is also preferred to use alginate as the first compound, an Enterobacterium such as Escherichia coli as the object to be attached, and poly-D-lysine as the second compound.

It is also preferred to use Nafion® as the first compound, an Enterobacterium such as Escherichia coli as the object to be attached, and poly-D-lysine as the second compound.

It is also preferred to use polydimethylsiloxane (PDMS) as the first compound, an Enterobacterium such as Escherichia coli as the object to be attached, and poly-D-lysine as the second compound.

It is also preferred to use polyethylene glycol (PEG) as the first compound, an Enterobacterium such as Escherichia coli as the object to be attached, and poly-D-lysine as the second compound.

It is furthermore preferred to use agarose as the first compound, an Enterobacterium such as Escherichia coli as the object to be attached, and laminin as the second compound.

It is also preferred to use agar as the first compound, an Enterobacterium such as Escherichia coli as the object to be attached, and laminin as the second compound.

It is also preferred to use alginate as the first compound, an Enterobacterium such as Escherichia coli as the object to be attached, and laminin as the second compound.

It is also preferred to use Nafion® as the first compound, an Enterobacterium such as Escherichia coli as the object to be attached, and laminin as the second compound.

It is also preferred to use polydimethylsiloxane (PDMS) as the first compound, an Enterobacterium such as Escherichia coli as the object to be attached, laminin as the second compound.

It is also preferred to use polyethylene glycol (PEG) as the first compound, an Enterobacterium such as Escherichia coli as the object to be attached, and laminin as the second compound.

It is furthermore preferred to use agarose as the first compound, an Enterobacterium such as Escherichia coli as the object to be attached, and chitosan as the second compound.

It is also preferred to use agar as the first compound, an Enterobacterium such as Escherichia coli as the object to be attached, and chitosan as the second compound.

It is also preferred to use alginate as the first compound, an Enterobacterium such as Escherichia coli as the object to be attached, and chitosan as the second compound.

It is also preferred to use Nafion® as the first compound, an Enterobacterium such as Escherichia coli as the object to be attached, and chitosan as the second compound.

It is also preferred to use polydimethylsiloxane (PDMS) as the first compound, an Enterobacterium such as Escherichia coli as the object to be attached, chitosan as the second compound.

It is also preferred to use polyethylene glycol (PEG) as the first compound, an Enterobacterium such as Escherichia coli as the object to be attached, and chitosan as the second compound.

It is furthermore preferred to use agarose as the first compound, an Enterobacterium such as Escherichia coli as the object to be attached, and glutaraldehyde as the second compound.

It is also preferred to use agar as the first compound, an Enterobacterium such as Escherichia coli as the object to be attached, and glutaraldehyde as the second compound.

It is also preferred to use alginate as the first compound, an Enterobacterium such as Escherichia coli as the object to be attached, and glutaraldehyde as the second compound.

It is also preferred to use Nafion® as the first compound, an Enterobacterium such as Escherichia coli as the object to be attached, and glutaraldehyde as the second compound.

It is also preferred to use polydimethylsiloxane (PDMS) as the first compound, an Enterobacterium such as Escherichia coli as the object to be attached, and glutaraldehyde as the second compound.

It is also preferred to use polyethylene glycol (PEG) as the first compound, an Enterobacterium such as Escherichia coli as the object to be attached, and glutaraldehyde as the second compound.

It is furthermore preferred to use agarose as the first compound, an Enterobacterium such as Escherichia coli as the object to be attached, and (3-Aminopropyl)triethoxysilane (APTES) as the second compound.

It is also preferred to use agar as the first compound, an Enterobacterium such as Escherichia coli as the object to be attached, and (3-Aminopropyl)triethoxysilane (APTES) as the second compound.

It is also preferred to use alginate as the first compound, an Enterobacterium such as Escherichia coli as the object to be attached, and (3-Aminopropyl)triethoxysilane (APTES) as the second compound.

It is also preferred to use Nafion® as the first compound, an Enterobacterium such as Escherichia coli as the object to be attached, and (3-Aminopropyl)triethoxysilane (APTES) as the second compound.

It is also preferred to use polydimethylsiloxane (PDMS) as the first compound, an Enterobacterium such as Escherichia coli as the object to be attached, and (3-Aminopropyl)triethoxysilane (APTES) as the second compound.

It is also preferred to use polyethylene glycol (PEG) as the first compound, an Enterobacterium such as Escherichia coli as the object to be attached, and (3-Aminopropyl)triethoxysilane (APTES) as the second compound.

It is furthermore preferred to use agarose as the first compound, an Enterobacterium such as Escherichia coli as the object to be attached, and poly(sodium-p-styrene sulfonate)/poly(allylamine hydrochloride) copolymer (PSS/PAH) as the second compound.

It is also preferred to use agar as the first compound, an Enterobacterium such as Escherichia coli as the object to be attached, and poly(sodium-p-styrene sulfonate)/poly(allylamine hydrochloride) copolymer (PSS/PAH) as the second compound.

It is also preferred to use alginate as the first compound, an Enterobacterium such as Escherichia coli as the object to be attached, and poly(sodium-p-styrene sulfonate)/poly(allylamine hydrochloride) copolymer (PSS/PAH) as the second compound.

It is also preferred to use Nafion® as the first compound, an Enterobacterium such as Escherichia coli as the object to be attached, and poly(sodium-p-styrene sulfonate)/poly(allylamine hydrochloride) copolymer (PSS/PAH) as the second compound.

It is also preferred to use polydimethylsiloxane (PDMS) as the first compound, an Enterobacterium such as Escherichia coli as the object to be attached, and poly(sodium-p-styrene sulfonate)/poly(allylamine hydrochloride) copolymer (PSS/PAH) as the second compound.

It is also preferred to use polyethylene glycol (PEG) as the first compound, an Enterobacterium such as Escherichia coli as the object to be attached, and poly(sodium-p-styrene sulfonate)/poly(allylamine hydrochloride) copolymer (PSS/PAH) as the second compound.

It is furthermore preferred to use agarose as the first compound, an Enterobacterium such as Escherichia coli as the object to be attached, and Mussel Adhesive recombinant protein (MAPTrix™) as the second compound.

It is also preferred to use agar as the first compound, an Enterobacterium such as Escherichia coli as the object to be attached, and Mussel Adhesive recombinant protein (MAPTrix™) as the second compound.

It is also preferred to use alginate as the first compound, an Enterobacterium such as Escherichia coli as the object to be attached, and Mussel Adhesive recombinant protein (MAPTrix™) as the second compound.

It is also preferred to use Nafion® as the first compound, an Enterobacterium such as Escherichia coli as the object to be attached, and Mussel Adhesive recombinant protein (MAPTrix™) as the second compound.

It is also preferred to use polydimethylsiloxane (PDMS) as the first compound, an Enterobacterium such as Escherichia coli as the object to be attached, and Mussel Adhesive recombinant protein (MAPTrix™) as the second compound.

It is also preferred to use polyethylene glycol (PEG) as the first compound, an Enterobacterium such as Escherichia coli as the object to be attached, and Mussel Adhesive recombinant protein (MAPTrix™) as the second compound.

It is furthermore preferred to use agarose as the first compound, an Enterobacterium such as Escherichia coli as the object to be attached, and 4-Aminothiophenol (4-ATP) as the second compound.

It is also preferred to use agar as the first compound, an Enterobacterium such as Escherichia coli as the object to be attached, and 4-Aminothiophenol (4-ATP) as the second compound.

It is also preferred to use alginate as the first compound, an Enterobacterium such as Escherichia coli as the object to be attached, and 4-Aminothiophenol (4-ATP) as the second compound.

It is also preferred to use Nafion® as the first compound, an Enterobacterium such as Escherichia coli as the object to be attached, and 4-Aminothiophenol (4-ATP) as the second compound.

It is also preferred to use polydimethylsiloxane (PDMS) as the first compound, an Enterobacterium such as Escherichia coli as the object to be attached, and 4-Aminothiophenol (4-ATP) as the second compound.

It is also preferred to use polyethylene glycol (PEG) as the first compound, an Enterobacterium such as Escherichia coli as the object to be attached, and 4-Aminothiophenol (4-ATP) as the second compound.

It is furthermore preferred to use agarose as the first compound, a Mycobacteria such as Mycobacterium smegmatis as the object to be attached, and poly-D-lysine as the second compound.

It is also preferred to use agar as the first compound, a Mycobacteria such as Mycobacterium smegmatis as the object to be attached, and poly-D-lysine as the second compound.

It is also preferred to use alginate as the first compound, a Mycobacteria such as Mycobacterium smegmatis as the object to be attached, and poly-D-lysine as the second compound.

It is also preferred to use Nafion® as the first compound, a Mycobacteria such as Mycobacterium smegmatis as the object to be attached, and poly-D-lysine as the second compound.

It is also preferred to use polydimethylsiloxane (PDMS) as the first compound, a Mycobacteria such as Mycobacterium smegmatis as the object to be attached, and poly-D-lysine as the second compound.

It is also preferred to use polyethylene glycol (PEG) as the first compound, a Mycobacteria such as Mycobacterium smegmatis as the object to be attached, and poly-D-lysine as the second compound.

It is furthermore preferred to use agarose as the first compound, a Mycobacteria such as Mycobacterium smegmatis as the object to be attached, and laminin as the second compound.

It is also preferred to use agar as the first compound, a Mycobacteria such as Mycobacterium smegmatis as the object to be attached, and laminin as the second compound.

It is also preferred to use alginate as the first compound, a Mycobacteria such as Mycobacterium smegmatis as the object to be attached, and laminin as the second compound.

It is also preferred to use Nafion® as the first compound, a Mycobacteria such as Mycobacterium smegmatis as the object to be attached, and laminin as the second compound.

It is also preferred to use polydimethylsiloxane (PDMS) as the first compound, a Mycobacteria such as Mycobacterium smegmatis as the object to be attached, laminin as the second compound.

It is also preferred to use polyethylene glycol (PEG) as the first compound, a Mycobacteria such as Mycobacterium smegmatis as the object to be attached, and laminin as the second compound.

It is furthermore preferred to use agarose as the first compound, a Mycobacteria such as Mycobacterium smegmatis as the object to be attached, and chitosan as the second compound.

It is also preferred to use agar as the first compound, a Mycobacteria such as Mycobacterium smegmatis as the object to be attached, and chitosan as the second compound.

It is also preferred to use alginate as the first compound, a Mycobacteria such as Mycobacterium smegmatis as the object to be attached, and chitosan as the second compound.

It is also preferred to use Nafion® as the first compound, a Mycobacteria such as Mycobacterium smegmatis as the object to be attached, and chitosan as the second compound.

It is also preferred to use polydimethylsiloxane (PDMS) as the first compound, a Mycobacteria such as Mycobacterium smegmatis as the object to be attached, chitosan as the second compound.

It is also preferred to use polyethylene glycol (PEG) as the first compound, a Mycobacteria such as Mycobacterium smegmatis as the object to be attached, and chitosan as the second compound.

It is furthermore preferred to use agarose as the first compound, a Mycobacteria such as Mycobacterium smegmatis as the object to be attached, and glutaraldehyde as the second compound.

It is also preferred to use agar as the first compound, a Mycobacteria such as Mycobacterium smegmatis as the object to be attached, and glutaraldehyde as the second compound.

It is also preferred to use alginate as the first compound, a Mycobacteria such as Mycobacterium smegmatis as the object to be attached, and glutaraldehyde as the second compound.

It is also preferred to use Nafion® as the first compound, a Mycobacteria such as Mycobacterium smegmatis as the object to be attached, and glutaraldehyde as the second compound.

It is also preferred to use polydimethylsiloxane (PDMS) as the first compound, a Mycobacteria such as Mycobacterium smegmatis as the object to be attached, and glutaraldehyde as the second compound.

It is also preferred to use polyethylene glycol (PEG) as the first compound, a Mycobacteria such as Mycobacterium smegmatis as the object to be attached, and glutaraldehyde as the second compound.

It is furthermore preferred to use agarose as the first compound, a Mycobacteria such as Mycobacterium smegmatis as the object to be attached, and (3-Aminopropyl)triethoxysilane (APTES) as the second compound.

It is also preferred to use agar as the first compound, a Mycobacteria such as Mycobacterium smegmatis as the object to be attached, and (3-Aminopropyl)triethoxysilane (APTES) as the second compound.

It is also preferred to use alginate as the first compound, a Mycobacteria such as Mycobacterium smegmatis as the object to be attached, and (3-Aminopropyl)triethoxysilane (APTES) as the second compound.

It is also preferred to use Nafion® as the first compound, a Mycobacteria such as Mycobacterium smegmatis as the object to be attached, and (3-Aminopropyl)triethoxysilane (APTES) as the second compound.

It is also preferred to use polydimethylsiloxane (PDMS) as the first compound, a Mycobacteria such as Mycobacterium smegmatis as the object to be attached, and (3-Aminopropyl)triethoxysilane (APTES) as the second compound.

It is also preferred to use polyethylene glycol (PEG) as the first compound, a Mycobacteria such as Mycobacterium smegmatis as the object to be attached, and (3-Aminopropyl)triethoxysilane (APTES) as the second compound.

It is furthermore preferred to use agarose as the first compound, a Mycobacteria such as Mycobacterium smegmatis as the object to be attached, and poly(sodium-p-styrene sulfonate)/poly(allylamine hydrochloride) copolymer (PSS/PAH) as the second compound.

It is also preferred to use agar as the first compound, a Mycobacteria such as Mycobacterium smegmatis as the object to be attached, and poly(sodium-p-styrene sulfonate)/poly(allylamine hydrochloride) copolymer (PSS/PAH) as the second compound.

It is also preferred to use alginate as the first compound, a Mycobacteria such as Mycobacterium smegmatis as the object to be attached, and poly(sodium-p-styrene sulfonate)/poly(allylamine hydrochloride) copolymer (PSS/PAH) as the second compound.

It is also preferred to use Nafion® as the first compound, a Mycobacteria such as Mycobacterium smegmatis as the object to be attached, and poly(sodium-p-styrene sulfonate)/poly(allylamine hydrochloride) copolymer (PSS/PAH) as the second compound.

It is also preferred to use polydimethylsiloxane (PDMS) as the first compound, a Mycobacteria such as Mycobacterium smegmatis as the object to be attached, and poly(sodium-p-styrene sulfonate)/poly(allylamine hydrochloride) copolymer (PSS/PAH) as the second compound.

It is also preferred to use polyethylene glycol (PEG) as the first compound, a Mycobacteria such as Mycobacterium smegmatis as the object to be attached, and poly(sodium-p-styrene sulfonate)/poly(allylamine hydrochloride) copolymer (PSS/PAH) as the second compound.

It is furthermore preferred to use agarose as the first compound, a Mycobacteria such as Mycobacterium smegmatis as the object to be attached, and Mussel Adhesive recombinant protein (MAPTrix™) as the second compound.

It is also preferred to use agar as the first compound, a Mycobacteria such as Mycobacterium smegmatis as the object to be attached, and Mussel Adhesive recombinant protein (MAPTrix™) as the second compound.

It is also preferred to use alginate as the first compound, a Mycobacteria such as Mycobacterium smegmatis as the object to be attached, and Mussel Adhesive recombinant protein (MAPTrix™) as the second compound.

It is also preferred to use Nafion® as the first compound, a Mycobacteria such as Mycobacterium smegmatis as the object to be attached, and Mussel Adhesive recombinant protein (MAPTrix™) as the second compound.

It is also preferred to use polydimethylsiloxane (PDMS) as the first compound, a Mycobacteria such as Mycobacterium smegmatis as the object to be attached, and Mussel Adhesive recombinant protein (MAPTrix™) as the second compound.

It is also preferred to use polyethylene glycol (PEG) as the first compound, a Mycobacteria such as Mycobacterium smegmatis as the object to be attached, and Mussel Adhesive recombinant protein (MAPTrix™) as the second compound.

It is furthermore preferred to use agarose as the first compound, a Mycobacteria such as Mycobacterium smegmatis as the object to be attached, and 4-Aminothiophenol (4-ATP) as the second compound.

It is also preferred to use agar as the first compound, a Mycobacteria such as Mycobacterium smegmatis as the object to be attached, and 4-Aminothiophenol (4-ATP) as the second compound.

It is also preferred to use alginate as the first compound, a Mycobacteria such as Mycobacterium smegmatis as the object to be attached, and 4-Aminothiophenol (4-ATP) as the second compound.

It is also preferred to use Nafion® as the first compound, a Mycobacteria such as Mycobacterium smegmatis as the object to be attached, and 4-Aminothiophenol (4-ATP) as the second compound.

It is also preferred to use polydimethylsiloxane (PDMS) as the first compound, a Mycobacteria such as Mycobacterium smegmatis as the object to be attached, and 4-Aminothiophenol (4-ATP) as the second compound.

It is also preferred to use polyethylene glycol (PEG) as the first compound, a Mycobacteria such as Mycobacterium smegmatis as the object to be attached, and 4-Aminothiophenol (4-ATP) as the second compound.

It is furthermore preferred to use agarose as the first compound, Mammalian cells such as Vero cells as the object to be attached, and poly-D-lysine as the second compound.

It is also preferred to use agar as the first compound, Mammalian cells such as Vero cells as the object to be attached, and poly-D-lysine as the second compound.

It is also preferred to use alginate as the first compound, Mammalian cells such as Vero cells as the object to be attached, and poly-D-lysine as the second compound.

It is also preferred to use Nafion® as the first compound, Mammalian cells such as Vero cells as the object to be attached, and poly-D-lysine as the second compound.

It is also preferred to use polydimethylsiloxane (PDMS) as the first compound, Mammalian cells such as Vero cells as the object to be attached, and poly-D-lysine as the second compound.

It is also preferred to use polyethylene glycol (PEG) as the first compound, Mammalian cells such as Vero cells as the object to be attached, and poly-D-lysine as the second compound.

It is furthermore preferred to use agarose as the first compound, Mammalian cells such as Vero cells as the object to be attached, and laminin as the second compound.

It is also preferred to use agar as the first compound, Mammalian cells such as Vero cells as the object to be attached, and laminin as the second compound.

It is also preferred to use alginate as the first compound, Mammalian cells such as Vero cells as the object to be attached, and laminin as the second compound.

It is also preferred to use Nafion® as the first compound, Mammalian cells such as Vero cells as the object to be attached, and laminin as the second compound.

It is also preferred to use polydimethylsiloxane (PDMS) as the first compound, Mammalian cells such as Vero cells as the object to be attached, laminin as the second compound.

It is also preferred to use polyethylene glycol (PEG) as the first compound, Mammalian cells such as Vero cells as the object to be attached, and laminin as the second compound.

It is furthermore preferred to use agarose as the first compound, Mammalian cells such as Vero cells as the object to be attached, and chitosan as the second compound.

It is also preferred to use agar as the first compound, Mammalian cells such as Vero cells as the object to be attached, and chitosan as the second compound.

It is also preferred to use alginate as the first compound, Mammalian cells such as Vero cells as the object to be attached, and chitosan as the second compound.

It is also preferred to use Nafion® as the first compound, Mammalian cells such as Vero cells as the object to be attached, and chitosan as the second compound.

It is also preferred to use polydimethylsiloxane (PDMS) as the first compound, Mammalian cells such as Vero cells as the object to be attached, chitosan as the second compound.

It is also preferred to use polyethylene glycol (PEG) as the first compound, Mammalian cells such as Vero cells as the object to be attached, and chitosan as the second compound.

It is furthermore preferred to use agarose as the first compound, Mammalian cells such as Vero cells as the object to be attached, and glutaraldehyde as the second compound.

It is also preferred to use agar as the first compound, Mammalian cells such as Vero cells as the object to be attached, and glutaraldehyde as the second compound.

It is also preferred to use alginate as the first compound, Mammalian cells such as Vero cells as the object to be attached, and glutaraldehyde as the second compound.

It is also preferred to use Nafion® as the first compound, Mammalian cells such as Vero cells as the object to be attached, and glutaraldehyde as the second compound.

It is also preferred to use polydimethylsiloxane (PDMS) as the first compound, Mammalian cells such as Vero cells as the object to be attached, and glutaraldehyde as the second compound.

It is also preferred to use polyethylene glycol (PEG) as the first compound, Mammalian cells such as Vero cells as the object to be attached, and glutaraldehyde as the second compound.

It is furthermore preferred to use agarose as the first compound, Mammalian cells such as Vero cells as the object to be attached, and (3-Aminopropyl)triethoxysilane (APTES) as the second compound.

It is also preferred to use agar as the first compound, Mammalian cells such as Vero cells as the object to be attached, and (3-Aminopropyl)triethoxysilane (APTES) as the second compound.

It is also preferred to use alginate as the first compound, Mammalian cells such as Vero cells as the object to be attached, and (3-Aminopropyl)triethoxysilane (APTES) as the second compound.

It is also preferred to use Nafion® as the first compound, Mammalian cells such as Vero cells as the object to be attached, and (3-Aminopropyl)triethoxysilane (APTES) as the second compound.

It is also preferred to use polydimethylsiloxane (PDMS) as the first compound, Mammalian cells such as Vero cells as the object to be attached, and (3-Aminopropyl)triethoxysilane (APTES) as the second compound.

It is also preferred to use polyethylene glycol (PEG) as the first compound, Mammalian cells such as Vero cells as the object to be attached, and (3-Aminopropyl)triethoxysilane (APTES) as the second compound.

It is furthermore preferred to use agarose as the first compound, Mammalian cells such as Vero cells as the object to be attached, and poly(sodium-p-styrene sulfonate)/poly(allylamine hydrochloride) copolymer (PSS/PAH) as the second compound.

It is also preferred to use agar as the first compound, Mammalian cells such as Vero cells as the object to be attached, and poly(sodium-p-styrene sulfonate)/poly(allylamine hydrochloride) copolymer (PSS/PAH) as the second compound.

It is also preferred to use alginate as the first compound, Mammalian cells such as Vero cells as the object to be attached, and poly(sodium-p-styrene sulfonate)/poly(allylamine hydrochloride) copolymer (PSS/PAH) as the second compound.

It is also preferred to use Nafion® as the first compound, Mammalian cells such as Vero cells as the object to be attached, and poly(sodium-p-styrene sulfonate)/poly(allylamine hydrochloride) copolymer (PSS/PAH) as the second compound.

It is also preferred to use polydimethylsiloxane (PDMS) as the first compound, Mammalian cells such as Vero cells as the object to be attached, and poly(sodium-p-styrene sulfonate)/poly(allylamine hydrochloride) copolymer (PSS/PAH) as the second compound.

It is also preferred to use polyethylene glycol (PEG) as the first compound, Mammalian cells such as Vero cells as the object to be attached, and poly(sodium-p-styrene sulfonate)/poly(allylamine hydrochloride) copolymer (PSS/PAH) as the second compound.

It is furthermore preferred to use agarose as the first compound, Mammalian cells such as Vero cells as the object to be attached, and Mussel Adhesive recombinant protein (MAPTrix™) as the second compound.

It is also preferred to use agar as the first compound, Mammalian cells such as Vero cells as the object to be attached, and Mussel Adhesive recombinant protein (MAPTrix™) as the second compound.

It is also preferred to use alginate as the first compound, Mammalian cells such as Vero cells as the object to be attached, and Mussel Adhesive recombinant protein (MAPTrix™) as the second compound.

It is also preferred to use Nafion® as the first compound, Mammalian cells such as Vero cells as the object to be attached, and Mussel Adhesive recombinant protein (MAPTrix™) as the second compound.

It is also preferred to use polydimethylsiloxane (PDMS) as the first compound, Mammalian cells such as Vero cells as the object to be attached, and Mussel Adhesive recombinant protein (MAPTrix™) as the second compound.

It is also preferred to use polyethylene glycol (PEG) as the first compound, Mammalian cells such as Vero cells as the object to be attached, and Mussel Adhesive recombinant protein (MAPTrix™) as the second compound.

It is furthermore preferred to use agarose as the first compound, Mammalian cells such as Vero cells as the object to be attached, and 4-Aminothiophenol (4-ATP) as the second compound.

It is also preferred to use agar as the first compound, Mammalian cells such as Vero cells as the object to be attached, and 4-Aminothiophenol (4-ATP) as the second compound.

It is also preferred to use alginate as the first compound, Mammalian cells such as Vero cells as the object to be attached, and 4-Aminothiophenol (4-ATP) as the second compound.

It is also preferred to use Nafion® as the first compound, Mammalian cells such as Vero cells as the object to be attached, and 4-Aminothiophenol (4-ATP) as the second compound.

It is also preferred to use polydimethylsiloxane (PDMS) as the first compound, Mammalian cells such as Vero cells as the object to be attached, and 4-Aminothiophenol (4-ATP) as the second compound.

It is also preferred to use polyethylene glycol (PEG) as the first compound, Mammalian cells such as Vero cells as the object to be attached, and 4-Aminothiophenol (4-ATP) as the second compound.

It is furthermore preferred to use agarose as the first compound, yeast such as Candida albicans as the object to be attached, and poly-D-lysine as the second compound.

It is also preferred to use agar as the first compound, yeast such as Candida albicans as the object to be attached, and poly-D-lysine as the second compound.

It is also preferred to use alginate as the first compound, yeast such as Candida albicans as the object to be attached, and poly-D-lysine as the second compound.

It is also preferred to use Nafion® as the first compound, yeast such as Candida albicans as the object to be attached, and poly-D-lysine as the second compound.

It is also preferred to use polydimethylsiloxane (PDMS) as the first compound, yeast such as Candida albicans as the object to be attached, and poly-D-lysine as the second compound.

It is also preferred to use polyethylene glycol (PEG) as the first compound, yeast such as Candida albicans as the object to be attached, and poly-D-lysine as the second compound.

It is furthermore preferred to use agarose as the first compound, yeast such as Candida albicans as the object to be attached, and laminin as the second compound.

It is also preferred to use agar as the first compound, yeast such as Candida albicans as the object to be attached, and laminin as the second compound.

It is also preferred to use alginate as the first compound, yeast such as Candida albicans as the object to be attached, and laminin as the second compound.

It is also preferred to use Nafion® as the first compound, yeast such as Candida albicans as the object to be attached, and laminin as the second compound.

It is also preferred to use polydimethylsiloxane (PDMS) as the first compound, yeast such as Candida albicans as the object to be attached, laminin as the second compound.

It is also preferred to use polyethylene glycol (PEG) as the first compound, yeast such as Candida albicans as the object to be attached, and laminin as the second compound.

It is furthermore preferred to use agarose as the first compound, yeast such as Candida albicans as the object to be attached, and chitosan as the second compound.

It is also preferred to use agar as the first compound, yeast such as Candida albicans as the object to be attached, and chitosan as the second compound.

It is also preferred to use alginate as the first compound, yeast such as Candida albicans as the object to be attached, and chitosan as the second compound.

It is also preferred to use Nafion® as the first compound, yeast such as Candida albicans as the object to be attached, and chitosan as the second compound.

It is also preferred to use polydimethylsiloxane (PDMS) as the first compound, yeast such as Candida albicans as the object to be attached, chitosan as the second compound.

It is also preferred to use polyethylene glycol (PEG) as the first compound, yeast such as Candida albicans as the object to be attached, and chitosan as the second compound.

It is furthermore preferred to use agarose as the first compound, yeast such as Candida albicans as the object to be attached, and glutaraldehyde as the second compound.

It is also preferred to use agar as the first compound, yeast such as Candida albicans as the object to be attached, and glutaraldehyde as the second compound.

It is also preferred to use alginate as the first compound, yeast such as Candida albicans as the object to be attached, and glutaraldehyde as the second compound.

It is also preferred to use Nafion® as the first compound, yeast such as Candida albicans as the object to be attached, and glutaraldehyde as the second compound.

It is also preferred to use polydimethylsiloxane (PDMS) as the first compound, yeast such as Candida albicans as the object to be attached, and glutaraldehyde as the second compound.

It is also preferred to use polyethylene glycol (PEG) as the first compound, yeast such as Candida albicans as the object to be attached, and glutaraldehyde as the second compound.

It is furthermore preferred to use agarose as the first compound, yeast such as Candida albicans as the object to be attached, and (3-Aminopropyl)triethoxysilane (APTES) as the second compound.

It is also preferred to use agar as the first compound, yeast such as Candida albicans as the object to be attached, and (3-Aminopropyl)triethoxysilane (APTES) as the second compound.

It is also preferred to use alginate as the first compound, yeast such as Candida albicans as the object to be attached, and (3-Aminopropyl)triethoxysilane (APTES) as the second compound.

It is also preferred to use Nafion® as the first compound, yeast such as Candida albicans as the object to be attached, and (3-Aminopropyl)triethoxysilane (APTES) as the second compound.

It is also preferred to use polydimethylsiloxane (PDMS) as the first compound, yeast such as Candida albicans as the object to be attached, and (3-Aminopropyl)triethoxysilane (APTES) as the second compound.

It is also preferred to use polyethylene glycol (PEG) as the first compound, yeast such as Candida albicans as the object to be attached, and (3-Aminopropyl)triethoxysilane (APTES) as the second compound.

It is furthermore preferred to use agarose as the first compound, yeast such as Candida albicans as the object to be attached, and poly(sodium-p-styrene sulfonate)/poly(allylamine hydrochloride) copolymer (PSS/PAH) as the second compound.

It is also preferred to use agar as the first compound, yeast such as Candida albicans as the object to be attached, and poly(sodium-p-styrene sulfonate)/poly(allylamine hydrochloride) copolymer (PSS/PAH) as the second compound.

It is also preferred to use alginate as the first compound, yeast such as Candida albicans as the object to be attached, and poly(sodium-p-styrene sulfonate)/poly(allylamine hydrochloride) copolymer (PSS/PAH) as the second compound.

It is also preferred to use Nafion® as the first compound, yeast such as Candida albicans as the object to be attached, and poly(sodium-p-styrene sulfonate)/poly(allylamine hydrochloride) copolymer (PSS/PAH) as the second compound.

It is also preferred to use polydimethylsiloxane (PDMS) as the first compound, yeast such as Candida albicans as the object to be attached, and poly(sodium-p-styrene sulfonate)/poly(allylamine hydrochloride) copolymer (PSS/PAH) as the second compound.

It is also preferred to use polyethylene glycol (PEG) as the first compound, yeast such as Candida albicans as the object to be attached, and poly(sodium-p-styrene sulfonate)/poly(allylamine hydrochloride) copolymer (PSS/PAH) as the second compound.

It is furthermore preferred to use agarose as the first compound, yeast such as Candida albicans as the object to be attached, and Mussel Adhesive recombinant protein (MAPTrix™) as the second compound.

It is also preferred to use agar as the first compound, yeast such as Candida albicans as the object to be attached, and Mussel Adhesive recombinant protein (MAPTrix™) as the second compound.

It is also preferred to use alginate as the first compound, yeast such as Candida albicans as the object to be attached, and Mussel Adhesive recombinant protein (MAPTrix™) as the second compound.

It is also preferred to use Nafion® as the first compound, yeast such as Candida albicans as the object to be attached, and Mussel Adhesive recombinant protein (MAPTrix™) as the second compound.

It is also preferred to use polydimethylsiloxane (PDMS) as the first compound, yeast such as Candida albicans as the object to be attached, and Mussel Adhesive recombinant protein (MAPTrix™) as the second compound.

It is also preferred to use polyethylene glycol (PEG) as the first compound, yeast such as Candida albicans as the object to be attached, and Mussel Adhesive recombinant protein (MAPTrix™) as the second compound.

It is furthermore preferred to use agarose as the first compound, yeast such as Candida albicans as the object to be attached, and 4-Aminothiophenol (4-ATP) as the second compound.

It is also preferred to use agar as the first compound, yeast such as Candida albicans as the object to be attached, and 4-Aminothiophenol (4-ATP) as the second compound.

It is also preferred to use alginate as the first compound, yeast such as Candida albicans as the object to be attached, and 4-Aminothiophenol (4-ATP) as the second compound.

It is also preferred to use Nafion® as the first compound, yeast such as Candida albicans as the object to be attached, and 4-Aminothiophenol (4-ATP) as the second compound.

It is also preferred to use polydimethylsiloxane (PDMS) as the first compound, yeast such as Candida albicans as the object to be attached, and 4-Aminothiophenol (4-ATP) as the second compound.

It is also preferred to use polyethylene glycol (PEG) as the first compound, yeast such as Candida albicans as the object to be attached, and 4-Aminothiophenol (4-ATP) as the second compound.

The first solution preferably is an aqueous solution such as water, preferably an aqueous solution that may further comprise a buffer and/or a preferably dissolved salt such as sodium chloride. The buffer can be any buffer known in the art, such as a phosphate buffered saline (PBS) or another buffered solution such as Tris buffer, etc. The first solution may further comprise a growth medium.

The first solution preferably has a pH-value in the range of about 5 to 9, more preferably about 6 to 8, particularly preferably about 7. Furthermore, a rinsing solution can be provided. Said rinsing solution is preferably used to rinse the substrate after the biological object and/or non-biological object has been attached to the substrate in order to remove any non-attached objects from the substrate. The rinsing solution preferably corresponds to an aqueous solution such as water, phosphate buffered saline (PBS) or other buffered solutions such as Tris buffer, etc.

The growth medium is preferably used during the culturing of the biological objects that shall be attached to the substrate. When doing so the growth medium is added to a solution comprising the biological objects. Subsequently, said solution is washed, preferably by using a suitable buffer being known in the art. Thereafter, the at least one first compound is added to said solution, whereby the above-mentioned first dispersion is formed. Said first dispersion then comprises the biological objects, the at least one first compound, and the buffer. Then, said first dispersion is added to the substrate in order to attach the biological objects to the substrate. In a last step, the substrate comprising the attached biological objects is rinsed by the rinsing solution.

If a first solution comprising a growth medium is used, a growth medium known in the art is preferred. This could be, for example, at least one of pancreatic digest of casein, peptic digest of animal tissue, acid hydrolysate of casein, yeast extract, beef extract, starch such as corn starch, tryptone, peptone, dextrose and agar.

The first compound of the first solution preferably has a concentration in the range of between 0.0001% by weight to 10% by weight with respect to a total volume of the first solution, preferably between 0.001% by weight to 5% by weight with respect to the total volume of the first solution, particularly preferably between 0.02% by weight to 1% by weight with respect to the total volume of the first solution. Additionally or alternatively, the first compound of the first solution preferably has a concentration in the range of between 0.0001% by volume to 10% by volume with respect to a total volume of the first solution, preferably between 0.001% by volume to 5% by volume with respect to the total volume of the first solution, particularly preferably between 0.02% by volume to 1% by volume with respect to the total volume of the first solution. Additionally or alternatively the first compound of the first solution is preferably added to the first solution at a temperature between −20° C. to 120° C., preferably between 0° C. to 100° C., particularly preferably between 10° C. and 40° C. Here, the expression “volume with respect to the total volume” means “volume of the pure first compound per total volume of the first solution”.

The second compound of the second solution preferably has a concentration in the range of between 0.0001% by weight to 50% by weight with respect to a total volume of the second solution, preferably between 0.001% by weight to 5% by weight with respect to the total volume of the second solution, particularly preferably between 0.01% by weight to 2% by weight with respect to the total volume of the second solution. The second compound of the second solution preferably has a concentration in the range of between 0.0001% by volume to 50% by volume with respect to a total volume of the second solution, preferably between 0.001% by volume to 5% by volume with respect to the total volume of the second solution, particularly preferably between 0.01% by volume to 2% by volume with respect to the total volume of the second solution. Additionally or alternatively the second compound of the second solution is preferably added to the second solution at a temperature between −100° C. to 500° C., preferably between 0° C. and 100° C., particularly preferably between 10° C. and 40° C. Here, the expression “volume with respect to the total volume” means “volume of the pure second compound per total volume of the second solution”.

As indicated earlier, the second compound can be added to the substrate directly, i.e. in the absence of a second solution, or it can be provided together with a second solution. A preferred second solution is water. In the former case it is conceivable to apply the second compound to the surface of the substrate in the form of an aerosol, for example. In the latter case, it is conceivable to add the second compound to the second solution under standard pressure. However, depending on the chemical or physical characteristics of the second compound and the second solution, respectively, it might be preferred to add the second compound to the second solution under pressure and/or at high temperatures. For example, second compounds that are difficult to dissolve such as chitosan or APTES can be dissolved in the second solution, for example in water, at a pressure of between about 200 bar and at a temperature of about 370° C. Furthermore, more hydrophobic substances such as 4-ATP or APTES dissolve better in molten solids such as naphthalene or ethylene glycol. In order to melt these solids, temperatures above their melting points have to be applied. In the present example, said temperature should be above 197° C. in the case of ethylene glycol and above 218° C. in the case of naphthalene. Additionally or alternatively the second solution can comprise or consist of water or one or more solvents or mixtures thereof. The organic solvents are known in the art and can correspond to, e.g. ethanol or acetone and the like. An organic solvent is preferred in the event that the second compound is a hydrophobic compound.

As mentioned initially, it is conceivable to functionalize two or more parts of the surface of the substrate. In particular, at least a second part of the surface of the substrate can be functionalized, wherein a further first dispersion of a biological object and/or of a non-biological object in a further first solution is added to the functionalized second part of the surface, wherein the further first solution comprises at least one further first compound that differs from the first compound of the first solution that is added to the functionalized first part of the surface. Additionally or alternatively the functionalization of said second part of the surface of the substrate can differ from the functionalization of the first part of the surface of the substrate.

That is to say, it is conceivable to provide two or more first dispersions comprising two or more first compounds that differ from one another. To this end said two or more first compounds can differ in their chemical compositions. However, it is likewise conceivable that said two or more first compounds are chemically identical but differ in their concentration, for example. Additionally or alternatively it is conceivable to functionalize two or more parts of the surface of the substrate differently. For example, a first part could be chemically functionalized whereas a second part could be physically functionalized. However, it is likewise conceivable that both parts are chemically functionalized, wherein different second compounds are used for the surface treatment, or that both parts are physically functionalized, wherein the dimensions of their surface structures differ from one another. This procedure enables one to determine optimal compounds or conditions for the attachment of the biological objects and/or the non-biological objects in a rapid manner.

The substrate can be a flexible support, preferably a cantilever, a fibre such as a hollow fibre or a glass fibre, a membrane, a wire, a sponge, a flexible electrode, an integrated circuit, and a tuning fork. However, it is likewise conceivable that the substrate is a rigid support such as a glass cover slide, a ceramic tile, a rigid electrode, and a culture dish, for example. Additionally or alternatively the substrate can comprise a silicone-compound such as silicone dioxide or elementary silicone, plastic, ceramic, ceramic-metallic blend, a metal, a metal oxide or sulphide, and carbon such as graphite or diamond. The substrate preferably corresponds to a cantilever comprising or consisting of glass or quartz or to a silicone-chip.

Additionally or alternatively, at least part of the surface of the substrate can be coated with a coating prior to the functionalization of the surface of the substrate. The coating preferably comprising or consisting of at least one of a noble metal such as gold, a metal oxide such as titanium dioxide, a transition metal such as palladium, and a non-metal compound such as a nitride compound. For example, it is conceivable that part of the surface of the substrate, in particular a tip of the cantilever, is coated with a coating. A metal coating, for example, renders a flexibility of the substrate “tunable” as said coating increases the flexibility. A metallic surface, for example, has a better reactivity with reactive residues such as thiols. Metallic surfaces render the substrate conductive, making it an electrode, i.e. a sensor for other reactions such as the determination of a pH-value or the detection of redox compounds such hydrogen gas, quinones and so forth. Metal oxides or sulphides also confer further chemical properties to the substrate. Titanium dioxide, for example, can act as a catalyst in combination with ultraviolet radiation to kill microbes. Metal sulphides such as molybdenum sulphide can act as catalyst for redox reactions. Furthermore, oxides provide reactive surfaces that improve an attachment of the biological object and/or the non-biological object.

In any case it is preferred that after attachment of the object to the substrate the substrate comprises a layered structure. In particular, it is preferred that at least a first layer is arranged on at least a part of the surface of the structure, wherein said first layer is formed from at least a first dispersion as described above with reference to the method of attaching an object on a surface of the substrate. That is, the first layer is preferably formed from the first dispersion comprising the object, the first solution and the first compound. In fact, by adding the first dispersion to the substrate and subsequently incubating the substrate, a first layer can be formed. Said first layer can be directly arranged on the surface of the substrate. However, it is likewise conceivable that said first layer is indirectly arranged on the surface of the substrate. In fact, in this latter case it is conceivable that the surface of the substrate is functionalized, and wherein the first layer is arranged on the functionalized surface. For example, the functionalization of the substrate could be provided by means of a second layer being arranged on the surface of the substrate, and wherein the first layer in turn is arranged on said second layer. The second layer could be provided by the second solution comprising the second compound as described above, and wherein said second solution is allowed to solidify after its addition to the surface. For example, a solidification of the second solution could be achieved by allowing the second solution to dry.

A thickness of the first layer preferably is in the nanometer range to micrometer range or larger. For example, the first layer could have a thickness of 100 nanometer or more, for example of 1000 nanometer or more. Other thicknesses are however likewise conceivable and depend on the particular first compound, the particular object (the size of the object) the amount of first compound, etc. being used.

A thickness of the second layer preferably is in the nanometer range or larger. For example, the second layer could have a thickness of 10 nanometer or more, for example of 100 nanometer or more. Here, too, it should be noted that other thicknesses are however likewise conceivable and depend on the particular second compound, the amount of the second compound, etc. being used.

An overall thickness encompassing the first layer, the second layer and the objects attached thereby is preferably in the micrometer range or larger.

In the following, preferred examples for the first solution and the second solution and their application during the attachment of a biological object on a substrate are given.

For example, the biological objects can correspond to Escherichia coli ATCC 25922 strain or to Klebsiella pneumoniae ATCC 27736 strain that are grown on Columbia medium (contains sheep blood) agar plates at 37° C.

The Columbia agar ingredients are the following:
Pancreatic digest of casein, 12.0 gram
Peptic digest of animal tissue, 5.0 gram
Yeast extract, 3.0 gram
Beef extract, 3.0 gram
Corn starch, 3.0 gram
Sodium chloride, 5.0 gram
Agar, 13.5 gram
Water, 1.0 litre.

The pH-value is 7.3±0.2.

Once per week the transferred strain is renewed to reduce the risk of mutations. That is, a frozen −80° C. culture is defrosted and plated on Columbia agar. Cells for attachment tests are taken from these plates, starting with the first plate-to-plate transfer. To harvest the cells, a fair amount of material is scraped off the agar surface using an inoculation loop and used to inoculate 3 ml of lysogeny broth (LB), see below. This step stimulates the activity of the cells and can be omitted if this is not necessary. After 20 minutes of incubation at 37° C., the cells are precipitated by centrifuging at 5,000 rpm and re-suspended in 1 ml PBS at pH 7.4. In other cases, longer stimulation periods are necessary. The cell material is then washed in PBS by centrifuging 4 times at 5,000 rounds per minute and re-suspending the cells again. After the fourth centrifuging, the cells are re-suspended in 200 microlitre PBS.

A different number of centrifuging steps may be used, depending the cell material and the medium. The optical density (OD, wavelength of 600 nm) of the washed suspension is between 1.0 and 1.3. This OD₆₀₀ corresponds to a McFarland standard between 8 and 15. To measure the corresponding McFarland turbidity, the cell suspension has to be diluted 1/10 in 0.85% NaCl. The OD may also be lower or higher or determined at a different wavelength. If a higher cell concentration is needed, the cell suspension is precipitated again and re-suspended in a lower volume of the buffer, for example in ⅕^{th}. Starting from the final dilution, cell forming units (CFU) are estimated using Mueller-Hinton agar plates as shown below. While in this example cells of E. coli are used, other cells, tissues, organisms, or viruses may be used as well. For example, yeasts such as Saccharomyces cerevisiae may be used. Cells of S. cerevisiae are grown on yeast extract peptone dextrose agar (YPD, see below) overnight. The cells are then stimulated for 2 h in the YPD medium and harvested in the same fashion as described above with reference to the E. coli ATCC 25922 strain.

The LB medium ingredients are the following:
Tryptone, 10.0 gram
Yeast extract, 5.0 gram
Sodium chloride, 10.0 gram
Water, 1.0 litre.

The pH-value is 7.0±0.2.

The Mueller-Hinton agar ingredients are the following:
Beef extract, 3.0 gram
Acid hydrolysate of casein, 17.5 gram
Starch, 1.5 gram
Water, 1.0 litre.

The pH-value is 7.3±0.1.

The yeast extract peptone dextrose agar (YPD) medium ingredients are the following:
Yeast extract, 2.0 gram
Peptone, 17.5 gram
Dextrose, 1.5 gram
Water, 1.0 litre.

The pH-value is 6.5±0.2.

### Example 1: The First Compound is Agar and the Second Compound is Glutaraldehyde

(1) Heat agar (2 g/l) in de-ionized (DI) water until it melts at 90-100° C.; prepare ˜1 millilitre liquid agar;
(2) Insert a substrate into a substrate holder and place the assembled piece on a layer of Parafilm® that adheres tightly to the inside of a plastic petri dish;
(3) Harvest and wash the biological objects to obtain a suspension of OD₆₀₀ being 1.0-1.3. If needed, a 5-fold concentrate of the biological objects can be used instead of an OD 1.0-1.3.
(4) To prepare the first solution, mix 800 microlitres of the biological object/PBS suspension (room temperature) with 200 microlitres of the 0.2% hot or cold liquid agar of step (1). The final agar concentration is 0.04%. Alternatively, the 0.2% or 0.001% agar solution can be prepared used.
(5) To prepare the second solution, dilute 25% glutaraldehyde to 0.5% in 0.85% sodium chloride;
(6) Place about 50 microlitres of the glutaraldehyde solution gently on the substrate to avoid breaking it. Incubate for 20 minutes at room temperature, optionally shake on an orbital shaker at 50 rounds per minute or optionally mix by pipetting up and down a few times every 2 minutes, and cover the dish to slow evaporation;
(7) Remove the glutaraldehyde solution of step (3) and rinse the substrate once with de-ionised water to remove any excess of the glutaraldehyde solution.
(8) A drop of the biological object/agar suspension is placed onto the substrate. The suspension on the functionalized substrate is incubated for 5 minutes at room temperature, optionally shaken on an orbital shaker at 50 rounds per minute or optionally mixing by pipetting up and down a few times every 2 minutes, and the dish is overed to slow evaporation.
(9) The biological object suspension is removed and the result is verified using a microscope. If desired, steps (8) and (9) are repeated.

### Example 2: The First Compound is Agar and the Second Compound is Chitosan

(1) To prepare a chitosan stock solution, dissolve 1 gram of chitosan (deacetylated crustacean chitin) in 100 millilitres 1% acetic acid solution by stirring overnight at room temperature;
(2) Filter the stock solution through 0.22 micrometer filters to remove remnant particles, store at 2-8° C.;
(3) Dilute the 1% chitosan stock solution to the desired final concentration using 0.85% NaCl, for example 1 milligram/millilitre will result in a coverage of 100 microgram/cm²
(4) Steps (1) to (9) of example 1 are carried out replacing the glutaraldehyde solution by the chitosan solution. Step (5) is omitted.

### Example 3: The First Compound is Agar and the Second Component is Poly-D-Lysine

(1) To prepare a stock solution, 10 milligrams poly-D-lysine (PDL) are dissolved in 1 millilitre DI water, filter-sterilized and stored at −20° C.;
(2) The 1% PDL stock solution is diluted to the desired final concentration using 0.85% NaCl, for example 0.1 milligram/millilitre (10 microgram/cm²);
(3) Steps (1) to (9) of example 1 are carried out replacing the glutaraldehyde solution by the PDL solution. Step (5) is omitted.

### Example 4: The First Compound is Agar and the Second Component is Cell-Tak™

(1) To obtain a stock solution of 50 microgram/millilitre, 22 microlitres of Cell-Tak™ in 778 microlitres 0.85% NaCl to obtain a stock solution of 50 microgram/millilitre (1.83 milligram/millitre are in the shipped product), store at −20° C. The stock solution corresponds to 7 micrograms/cm²;
(2) Dilute the stock solution to the desired concentration if necessary;
(3) Steps (1) to (9) of example 1 are carried out replacing the glutaraldehyde solution by the Cell-Tak™ solution. Step (5) is omitted.

### Example 5: The First Compound is Agar and the Second Component is BACproll®

(1) Two hundred microlitres of the BACproll® polymer solution is mixed with 200 microlitres of the reaction buffer;
(2) The stock solution is diluted to the desired concentration if necessary;
(3) Steps (1) to (9) of example 1 are carried out replacing the glutaraldehyde solution by the BACproll® solution. Step (5) is omitted.
BACproll® was used from the commercially available kit from Nittobo: https://nittobo-nmd.co.jp/english/special/rapidBACpro.html

### Example 6: The First Compound is Agar and the Second Component is APTES

(1) One hundred microlitres of APTES are mixed with 900 microlitres of de-ionised water to obtain a final concentration of 10%;
(2) Steps (1) to (9) of example 1 are carried out replacing the glutaraldehyde solution by the APTES solution. Step (5) is omitted.

### Example 7: The First Compound is Agar and the Second Component is 4-ATP

(1) To obtain a concentration of 10 mM, 4-ATP is dissolved in 1 millilitre of water acidified with HCl (pH=2) while stirring at 60° C. for 3 h;
(2) Steps (1) to (9) of example 1 are carried out replacing the glutaraldehyde solution by the 4-ATP solution. Omit step (5).

The first compound and its usage in the above examples 1 to 7 can be replaced by a first compound according to one of the below examples 8 to 11.

### Example 8: The First Compound is a Silicone Defoamer

(1) Replacing agar, 200 microlitres of a 25% Si defoamer stock solution is heated to 95° C. and added hot to 800 microlitres cell suspension in PBS.

In one example polydimethylsiloxane (PDMS) was used as silicone defoamer.

### Example 9: The First Compound is Alginate

(1) To prepare a stock solution, 1% (w/v) alginate is dissolved in DI water and filtered through a 0.22 micrometer membrane filter;
(2) Replacing agar, 50 microlitres of 1% alginate solution and 10 microlitres of 100 mM of a CaCl₂) solution are mixed with 940 microlitres of the cell suspension suspension

### Example 10: The First Compound is Polyacrylamide

(1) A polyacrylamide stock solution is diluted to obtain a final concentration 0.2% according to the protocol of the manufacturer;
(2) Replacing agar, 200 microlitres of the actively polymerizing solution are added to 200 microlitres of the cell suspension.

### Example 11: The First Compound is Nafion™

(1) Add 50 microlitres of a 5% (w/v) Nafion™ solution to 950 microlitres cell suspension. The final concentration will be 0.25% (w/v). If a dilution of Nafion® is needed, dilute 260 microlitres of 2-propanol or ethanol with 540 microlitres of a 5% (w/w) ethanesulfonyl fluoride polymer solution. The final concentration will be 0.03%.

In another example, Nafion™ was not diluted prior to the addition of the cell suspension. Instead, Nafion™ was diluted to a dispersion having a final concentration of 0.25% (w/v) Nafion with the cell suspension. In this case, water served as the solvent instead of 2-propanol or ethanol.

### Example 12: The First Compound is Agarose and the Second Compound is Poly-D-Lysine

(1) Heat agarose (2 g/l) in de-ionized (DI) water until it melts at 90-100° C.; prepare ˜1 millilitre liquid agarose;
(2) Insert a substrate into a substrate holder and place the assembled piece on a layer of Parafilm® that adheres tightly to the inside of a plastic petri dish;
(3) Harvest and wash the biological objects to obtain a suspension of OD₆₀₀ being 1.0-1.3. If needed, a 5-fold concentrate of the biological objects can be used instead of an OD 1.0-1.3.
(4) To prepare the first solution, mix 800 microlitres of the biological object/PBS suspension (room temperature) with 200 microlitres of the 0.2% hot or cold liquid agar of step (1). The final agarose concentration is 0.04%.
(5) To prepare the second solution, dissolve 10 milligram poly-D-lysine in 1 millilitre deionized water, filter-sterilize said second solution and store it at −20° C. This 1% stock solution is then diluted to the desired final concentration using 0.85% sodium chloride solution, for example 0.11 milligram/millilitre sodium chloride (or 10 microgram/cm² sodium chloride);
(6) Place about 50 microlitres of the poly-D-lysine solution gently on the substrate to avoid breaking it. Incubate for 20 minutes at room temperature, and cover the dish to slow evaporation;
(7) Remove the poly-D-lysine solution of step (3) and rinse the substrate once with de-ionised water to remove any excess of the poly-D-lysine solution.
(8) A drop of the biological object/agarose suspension is placed onto the substrate. The suspension on the functionalized substrate is incubated for 5 minutes at room temperature, mixed by pipetting up and down a few times every 2 minutes, and the dish is covered to slow evaporation.

The biological object suspension is removed and the result is verified using a microscope. If desired, steps (7) and (8) are repeated.

### Example 13: The First Compound is Agar and the Second Component is APTES

(1) Ten microlitres of APTES are mixed with 900 microlitres of ethanol to obtain a final concentration of 1%; The final stock solution of APTES was made of: 1% APTES, 94% of absolute ethanol and 5% of de-ionized water;
(2) Steps (1) to (8) of example 12 are carried out replacing the poly-D-lysine solution by the APTES solution.

### Example 14: The First Compound is Agar and the Second Component is 4-ATP

(1) To obtain a concentration of 10 mM, 125.19 milligrams of 4-ATP (125.19 Da) is dissolved in 100 millilitres ethanol;
(2) Steps (1) to (8) of example 12 are carried out replacing the poly-D-lysine solution by the 4-ATP solution.

### Example 15: The First Compound is Alginate

(1) To prepare a 1% (w/v) stock solution, 1 gram alginate is dissolved in 100 millilitres DI water and filtered through a 0.22 micrometer membrane filter;
(2) Replacing agar in Example 1, 250 microlitre of the 1% stock solution was mixed with 750 microlitres of the cell suspension. The final alginate concentration is 0.25%.

### BRIEF DESCRIPTION OF THE DRAWINGS

Preferred embodiments of the invention are described in the following with reference to the drawings, which are for the purpose of illustrating the present preferred embodiments of the invention and not for the purpose of limiting the same. In the drawings,

FIG. 1 shows a top view on a substrate according to a first embodiment;

FIG. 2 shows a perspective view on a substrate according to a further embodiment, wherein the substrate is attached to a mount;

FIG. 3a shows a side view of the substrate according to FIG. 2;

FIG. 3b shows a top view of the substrate according to FIG. 2;

FIG. 4 shows a top view of a substrate according to a further embodiment;

FIG. 5 shows a top view of a substrate according to a further embodiment;

FIG. 6 shows a top view of a substrate according to a further embodiment;

FIG. 7a shows a photograph of a substrate that has been treated with glutaraldehyde, and that has been subjected to E. coli B1;

FIG. 7b shows a photograph of a substrate that has been treated with glutaraldehyde, and that has been subjected to E. coli B1 submerged in an agar solution;

FIG. 8a shows a photograph of a substrate that has been treated with poly-D-lysine, and that has been subjected to E. coli ATCC 25922;

FIG. 8b shows a photograph of a substrate that has been treated with poly-D-lysine, and that has been subjected to E. coli ATCC 25922 submerged in an agar solution;

FIG. 9a shows a photograph of a substrate that has been treated with glutaraldehyde and that has been subjected to the E. coli resistant strain B1;

FIG. 9b shows a photograph of a substrate that has been treated with poly-D-lysine and that has been subjected to the E. coli resistant strain B1 submerged in an agar solution;

FIG. 9c shows a photograph of the substrate according to FIG. 9b after an incubation time of three hours;

FIG. 10 shows a photograph of an untreated substrate, and that has been subjected to E. coli ATCC 25922;

FIG. 11 shows a photograph of an untreated substrate, and that has been subjected to E. coli ATCC 25922 submerged in an agar solution;

FIG. 12 shows a photograph of a substrate that has been treated with glutaraldehyde, and that has been subjected to E. coli ATCC 25922;

FIG. 13 shows a photograph of a substrate that has been treated with glutaraldehyde, and that has been subjected to E. coli ATCC 25922, submerged in an agar solution;

FIG. 14 shows a photograph of a substrate that has been treated with poly-D-lysine, and that has been subjected to E. coli ATCC 25922;

FIG. 15 shows a photograph of a substrate that has been treated with poly-D-lysine, and that has been subjected to E. coli ATCC 25922 submerged in an agar solution;

FIG. 16 shows a photograph of an untreated substrate, and that has been subjected to E. coli ATCC 25922 submerged in a Nafion® solution;

FIG. 17 shows a photograph of a substrate that has been treated with glutaraldehyde, and that has been subjected to E. coli ATCC 25922 submerged in a Nafion® solution;

FIG. 18 shows a photograph of a substrate that has been treated with chitosan, and that has been subjected to E. coli ATCC 25922 submerged in a Nafion® solution;

FIG. 19 shows a photograph of an untreated substrate, and that has been subjected to E. coli ATCC 25922 submerged in a polyacrylamide solution,

FIG. 20 shows a photograph of a substrate that has been treated with glutaraldehyde, and that has been subjected to E. coli ATCC 25922 submerged in a polyacrylamide solution;

FIG. 21 shows a photograph of a substrate that has been treated with chitosan, and that has been subjected to E. coli ATCC 25922 submerged in a polyacrylamide solution;

FIG. 22a-22f show photographs of a substrate that has been treated with poly-D-lysine and that has been subjected to E. coli ATCC 25922 submerged in an agarose solution (a), in an agar solution (b), in an alginate solution (c), in a Nafion® solution (d), in a polydimethylsiloxane solution (e), and in a polyethylene glycol solution (f);

FIG. 23a-23g show photographs of a substrate that has been treated with laminin and that has been subjected to E. coli ATCC 25922 submerged in an agarose solution (a), in an agar solution (b), in an alginate solution (c), in a Nafion® solution (d), in a polydimethylsiloxane solution (e), and in a polyethylene glycol solution (f) as well as a photograph of a substrate that has been treated with laminin and that has been subjected to an E. coli ATCC 25922 suspension (g);

FIG. 24a-24g show photographs of a substrate that has been treated with chitosan and that has been subjected to E. coli ATCC 25922 submerged in an agarose solution (a), in an agar solution (b), in an alginate solution (c), in a Nafion® solution (d), in a polydimethylsiloxane solution (e), and in a polyethylene glycol solution (f) as well as a photograph of a substrate that has been treated with chitosan and that has been subjected to an E. coli ATCC 25922 suspension (g);

FIG. 25a-25f show photographs of a substrate that has been treated with glutaraldehyde and that has been subjected to E. coli ATCC 25922 submerged in an agarose solution (a), in an agar solution (b), in an alginate solution (c), in a Nafion® solution (d), in a polydimethylsiloxane solution (e), and in a polyethylene glycol solution (f);

FIG. 26a-26g show photographs of a substrate that has been treated with (3-aminopropyl)triethoxysilane (APTES) and that has been subjected to E. coli ATCC 25922 submerged in an agarose solution (a), in an agar solution (b), in an alginate solution (c), in a Nafion® solution (d), in a polydimethylsiloxane solution (e), and in a polyethylene glycol solution (f) as well as a photograph of a substrate that has been treated with (3-aminopropyl)triethoxysilane and that has been subjected to an E. coli ATCC 25922 suspension (g);

FIG. 27a-27f show photographs of a substrate that has been treated with poly(sodium-p-styrene sulfonate)/poly(allylamine hydrochloride) copolymer and that has been subjected to E. coli ATCC 25922 submerged in an agarose solution (a), in an agar solution (b), in an alginate solution (c), in a Nafion® solution (d), in a polydimethylsiloxane solution (e), and in a polyethylene glycol solution (f);

FIG. 28a-28g show photographs of a substrate that has been treated with MAPTrix™ and that has been subjected to E. coli ATCC 25922 submerged in an agarose solution (a), in an agar solution (b), in an alginate solution (c), in a Nafion® solution (d), in a polydimethylsiloxane solution (e), and in a polyethylene glycol solution (f) as well as a photograph of a substrate that has been treated with MAPTrix™ and that has been subjected to an E. coli ATCC 25922 suspension (g);

FIG. 29a-29g show photographs of a substrate that has been treated with 4-aminothiophenol and that has been subjected to E. coli ATCC 25922 submerged in an agarose solution (a), in an agar solution (b), in an alginate solution (c), in a Nafion® solution (d), in a polydimethylsiloxane solution (e), and in a polyethylene glycol solution (f) as well as a photograph of a substrate that has been treated with 4-aminothiophenol and that has been subjected to an E. coli ATCC 25922 suspension (g);

FIG. 30a-30g show photographs of an untreated substrate that has been has been subjected to E. coli ATCC 25922 submerged in an agarose solution (a), in an agar solution (b), in an alginate solution (c), in a Nafion® solution (d), in a polydimethylsiloxane solution (e), and in a polyethylene glycol solution (f) as well as a photograph of an untreated substrate that has been been subjected to an E. coli ATCC 25922 suspension (g);

FIG. 31a-31f show photographs of a substrate that has been treated with poly-D-lysine and that has been subjected to Mycobacterium smegmatis MC(2)155 submerged in an agar solution (a) and in an agarose solution (b), as well as a photograph of a substrate that has been treated with poly-D-lysine and that has been subjected to a Mycobacterium smegmatis MC(2)155 suspension (c), as well as photographs of an untreated substrate that has been subjected to Mycobacterium smegmatis MC(2)155 submerged in an agar solution (d) and in an agarose solution (e), as well as a photograph of an untreated substrate that has been subjected to a Mycobacterium smegmatis MC(2)155 suspension (f);

FIG. 32a-32f show photographs of a substrate that has been treated with poly-D-lysine and that has been subjected to Vero ATCC CCL-81 submerged in an agar solution (a) and in an agarose solution (b), as well as a photograph of a substrate that has been treated with poly-D-lysine and that has been subjected to a Vero ATCC CCL-81 suspension (c), as well as photographs of an untreated substrate that has been subjected to Vero ATCC CCL-81 submerged in an agar solution (d) and in an agarose solution (e), as well as a photograph of an untreated substrate that has been subjected to a Vero ATCC CCL-81 suspension (f);

FIG. 33a-33g show photographs of a substrate that has been treated with poly-D-lysine and that has been subjected to Candida albicans SC5314 submerged in an agarose solution (a), in an agar solution (b), in an alginate solution (c), in a Nafion® solution (d), in a polydimethylsiloxane solution (e), and in a polyethylene glycol solution (f) as well as a photograph of a substrate that has been treated with poly-D-lysine and that has been subjected to a Candida albicans SC5314 suspension (g);

FIG. 34a-34g show photographs of a substrate that has been treated with laminin and that has been subjected to Candida albicans SC5314 submerged in an agarose solution (a), in an agar solution (b), in an alginate solution (c), in a Nafion® solution (d), in a polydimethylsiloxane solution (e), and in a polyethylene glycol solution (f) as well as a photograph of a substrate that has been treated with laminin and that has been subjected to a Candida albicans SC5314 suspension (g);

FIG. 35a-35g show photographs of a substrate that has been treated with chitosan and that has been subjected to Candida albicans SC5314 submerged in an agarose solution (a), in an agar solution (b), in an alginate solution (c), in a Nafion® solution (d), in a polydimethylsiloxane solution (e), and in a polyethylene glycol solution (f) as well as a photograph of a substrate that has been treated with chitosan and that has been subjected to a Candida albicans SC5314 suspension (g);

FIG. 36a-36f show photographs of a substrate that has been treated with glutaraldehyde and that has been subjected to Candida albicans SC5314 submerged in an agarose solution (a), in an agar solution (b), in an alginate solution (c), in a Nafion® solution (d), in a polydimethylsiloxane solution (e), and in a polyethylene glycol solution (f);

FIG. 37a-37f show photographs of a substrate that has been treated with (3-aminopropyl)triethoxysilane and that has been subjected to Candida albicans SC5314 submerged in an agarose solution (a), in an agar solution (b), in an alginate solution (c), in a Nafion® solution (d), in a polydimethylsiloxane solution (e), and in a polyethylene glycol solution (f);

FIG. 38a-38f show photographs of a substrate that has been treated with poly(sodium-p-styrene sulfonate)/poly(allylamine hydrochloride copolymer and that has been subjected to Candida albicans SC5314 submerged in an agarose solution (a), in an agar solution (b), in an alginate solution (c), in a Nafion® solution (d), in a polydimethylsiloxane solution (e), and in a polyethylene glycol solution (f);

FIG. 39a-39f show photographs of a substrate that has been treated with MAPTrix™ and that has been subjected to Candida albicans SC5314 submerged in an agarose solution (a), in an agar solution (b), in an alginate solution (c), in a Nafion® solution (d), in a polydimethylsiloxane solution (e), and in a polyethylene glycol solution (f);

FIG. 40a-40f show photographs of a substrate that has been treated with 4-aminothiophenol and that has been subjected to Candida albicans SC5314 submerged in an agarose solution (a), in an agar solution (b), in an alginate solution (c), in a Nafion® solution (d), in a polydimethylsiloxane solution (e), and in a polyethylene glycol solution (f);

FIG. 41a-41g show photographs of an untreated substrate that has been has been subjected to Candida albicans SC5314 submerged in an agarose solution (a), in an agar solution (b), in an alginate solution (c), in a Nafion® solution (d), in a polydimethylsiloxane solution (e), and in a polyethylene glycol solution (f) as well as a photograph of an untreated substrate that has been been subjected to an Candida albicans SC5314 suspension (g);

FIG. 42a shows an image of an untreated substrate being attached to a mount recorded with an electron microscope;

FIG. 42b shows another image of the untreated substrate being attached to the mount according to FIG. 42a recorded with an electron microscope;

FIG. 43 shows an image of a substrate that has been treated with poly-D-lysine recorded with an electron microscope;

FIG. 44 shows an image of a substrate that has been treated with poly-D-lysine in a first step and that has been treated with an agarose solution in a subsequent step recorded with an electron microscope;

FIG. 45a-45f show images of a substrate that has been treated with poly-D-lysine in a first step and that has been treated with E. coli ATCC 25922 submerged in an agarose solution in a subsequent step recorded with an electron microscope.

### DESCRIPTION OF PREFERRED EMBODIMENTS

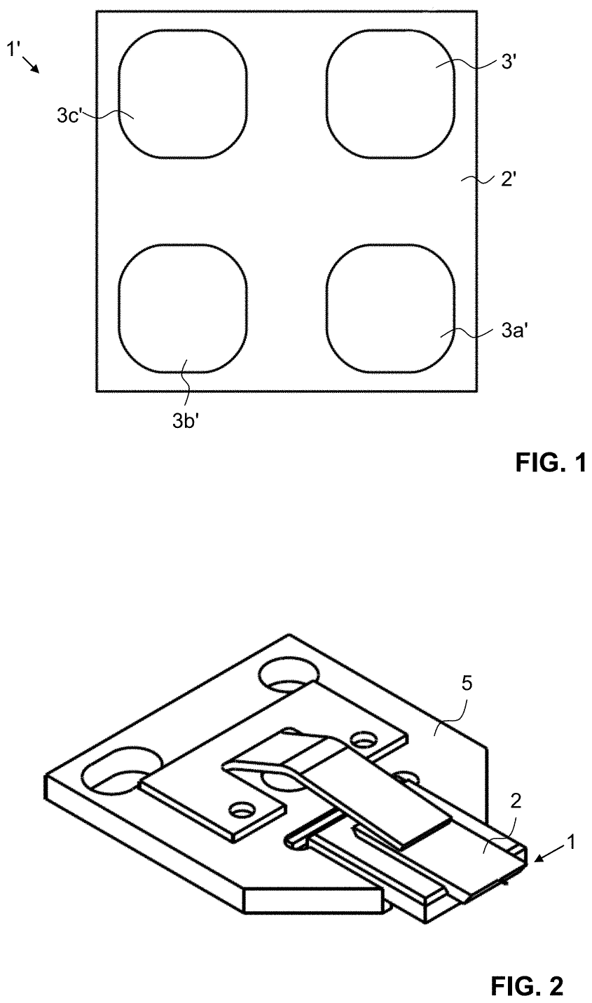

FIGS. 1 to 6 depict different embodiments of a substrate 1, 1′ for attaching biological objects and/or non-biological objects for illustrative purposes.

In fact, the substrate 1′ depicted in FIG. 1 corresponds to a rigid support in the form of a glass cover slide. Four parts 3′, 3a′, 3b′, 3c′ of a top surface 2′ of the glass cover slide 1 have been functionalized so as to allow an attachment of biological objects and/or of non-biological objects at four different conditions at a time. To this end the four parts 3′, 3a′, 3b′, 3c′ of the surface 2′ of the glass cover slides 1′ have been subjected to four different second compounds. For example, said different second compounds can correspond to different concentrations of the same second compound, different mixtures of different second compounds, or different incubation times, depending on the investigator's interest. In other words, FIG. 1 illustrates different chemical functionalizations of the surface 3′, 3a′, 3b′, 3c′ of the substrate 1′.

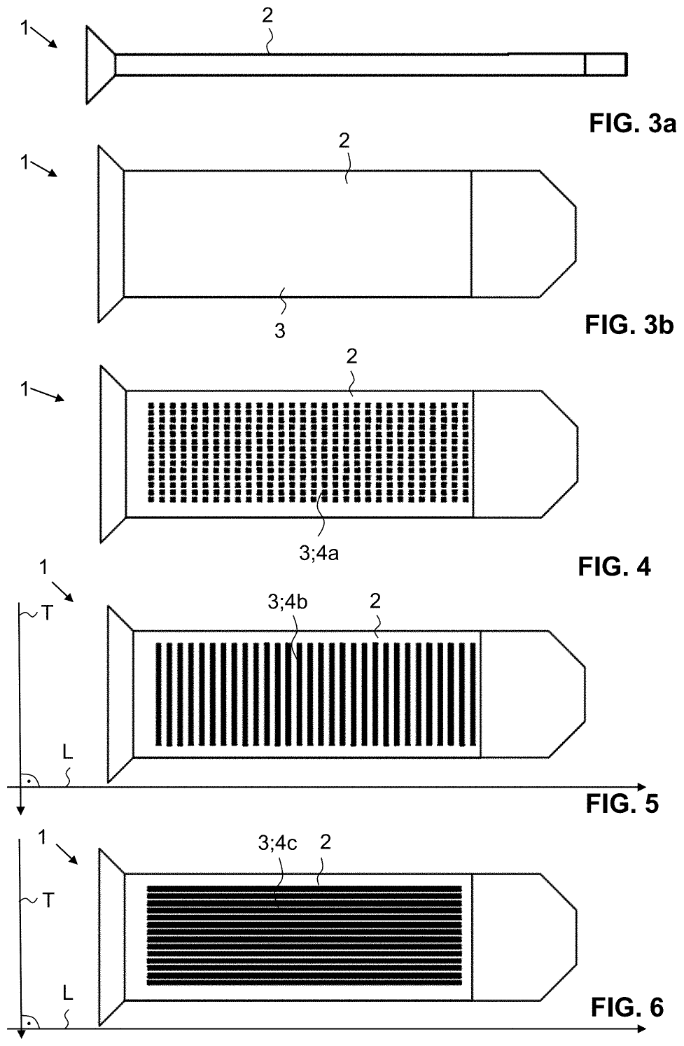

As has already been mentioned in the introduction, the attachment of biological objects is of great interest in the field of nanomotion AST, for example. In doing so the biological objects can be attached to a flexible support such as a cantilever, and wherein the movements of the cantilever that are caused by the attached biological objects are measured. FIGS. 2 to 6 depict different embodiments of a flexible substrate 1 in the form of a cantilever according to the invention which have proven to be very suitable and effective in these types of measurements. In fact, the cantilevers 1 can simply be attached to a mount 5 and be subject to measurements as disclosed in EP 2 766 722 B1, for example. The cantilevers 1 depicted in FIGS. 3a to 6 correspond here to essentially rectangular substrates that are etched from silicone wafers. The cantilevers 1 according to FIGS. 3a and 3b have in each case a surface 2 that has been chemically functionalized, wherein one or more second compounds preferably in a second solution have been added to the surface 2 of the cantilever, and wherein said one or more second compounds interact with the surface 2 of the cantilever, whereby the chemically functionalized surface 3 is formed. This is in contrast to the physically functionalized cantilevers 1 according to FIGS. 4 to 6, wherein a surface structure 4a, 4b, 4c has been generated in the surface 2 of the cantilever 1, whereby the physically functionalized surfaces 3 are formed. In particular, the cantilever 1 according to FIG. 4 comprises a surface structure 4a in the form of a dotted pattern, wherein the dots are recesses that reach into the surface 2 of the cantilever 1. The surface structures 4b, 4c of the cantilevers 1 depicted in FIGS. 5 and 6 correspond to a striped pattern, wherein the stripes are recesses that reach into the surface 2 of the cantilever 1. The stripes 4b of the cantilever 1 according to FIG. 5 extend along a transverse direction T of the cantilever 1, and the stripes 4c of the cantilever 1 according to FIG. 6 extend along a longitudinal direction L of the cantilever 1 that runs perpendicularly to the transverse direction T. These patterns 4a, 4b, 4c confer a surface topography to the cantilever 1 and are generated here by means of a KOH etching process.

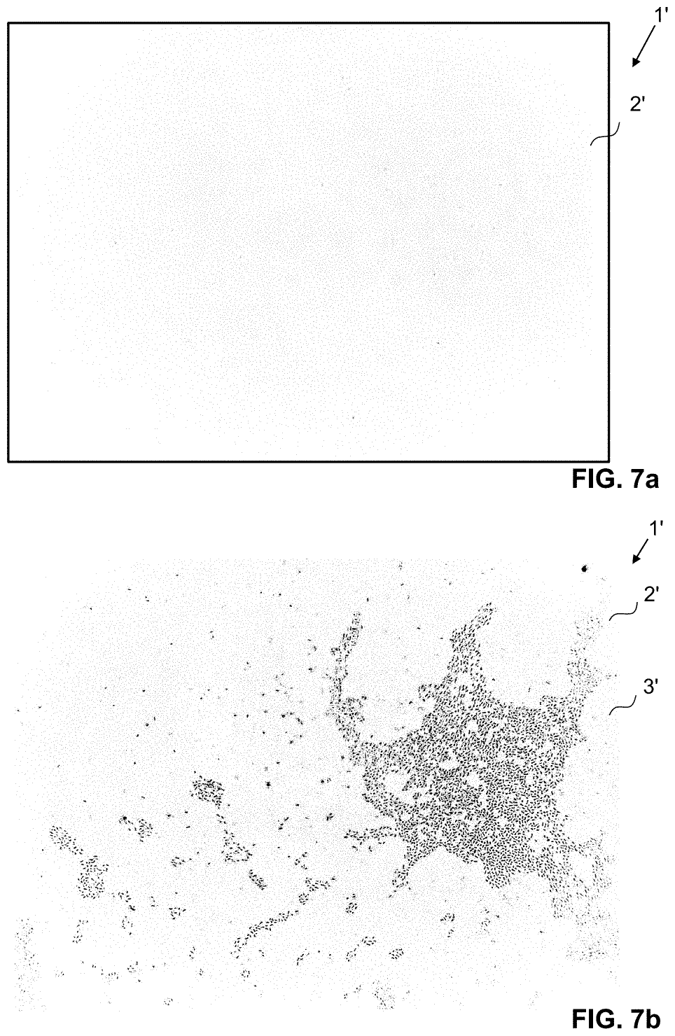

As has already been discussed in great detail above, the inventors have found out that the use of a first solution comprising at least one of a gelling agent, gellable agent, and a thickening agent in combination with a functionalized surface 3, 3′ of the substrate 1, 1′ results in an improved attachment of the biological object as compared to the attachment methods known in the state of the art. FIGS. 7a to 21 shall illustrate this effect.

Namely, FIGS. 7a and 7b depict photographs of an attached E. coli ceftriaxone resistant strain B1. The substrate 1′ in these figures corresponds in each case to a glass slide cover. The substrate 1′ depicted in FIG. 7a has been treated with a dispersion comprising a solution of 0.5% glutaraldehyde by weight per total volume of the solution and a suspension comprising the E. coli ceftriaxone resistant strain B1. The substrate 1′ depicted in FIG. 7b, however, has been functionalized with a dispersion comprising a solution of 0.5% glutaraldehyde by weight per total volume of the solution in a first step. The same surface has subsequently been treated with a solution comprising 0.04% agar by weight per total volume of the solution and the E. coli ceftriaxone resistant strain B1. As is readily evident from a comparison between FIGS. 7a and 7b, a much higher number of E. coli bacteria is attached on the substrate 1′ according to the invention and as depicted in FIG. 7b.

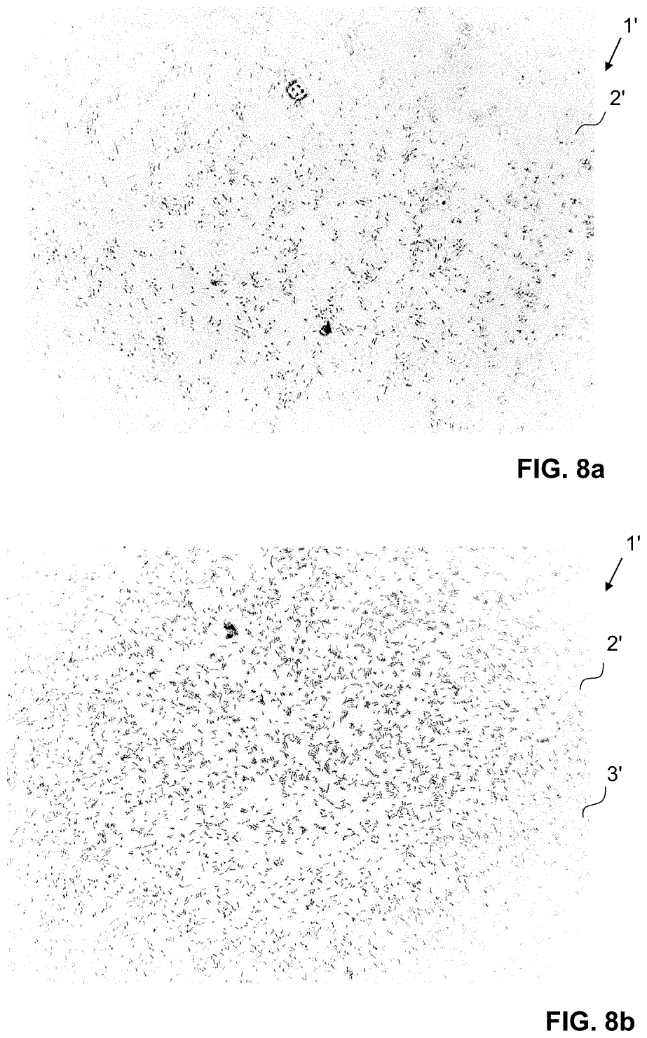

The same findings are found with respect to FIGS. 8a and 8b. Namely, FIG. 8a depicts a photograph of a substrate 1′ that has been functionalized with a dispersion comprising a solution of 0.1% poly-D-lysine by weight and subsequently with E. coli susceptible strain ATCC 25922. The substrate 1′ depicted in FIG. 8b, has been functionalized with a solution of 0.1% poly-D-lysine by weight. In a subsequent step, however the surface 2′ of the substrate 1′ of FIG. 8b has been with a solution comprising 0.04% agar by weight per total volume of the solution and E. coli susceptible strain ATCC 25922.

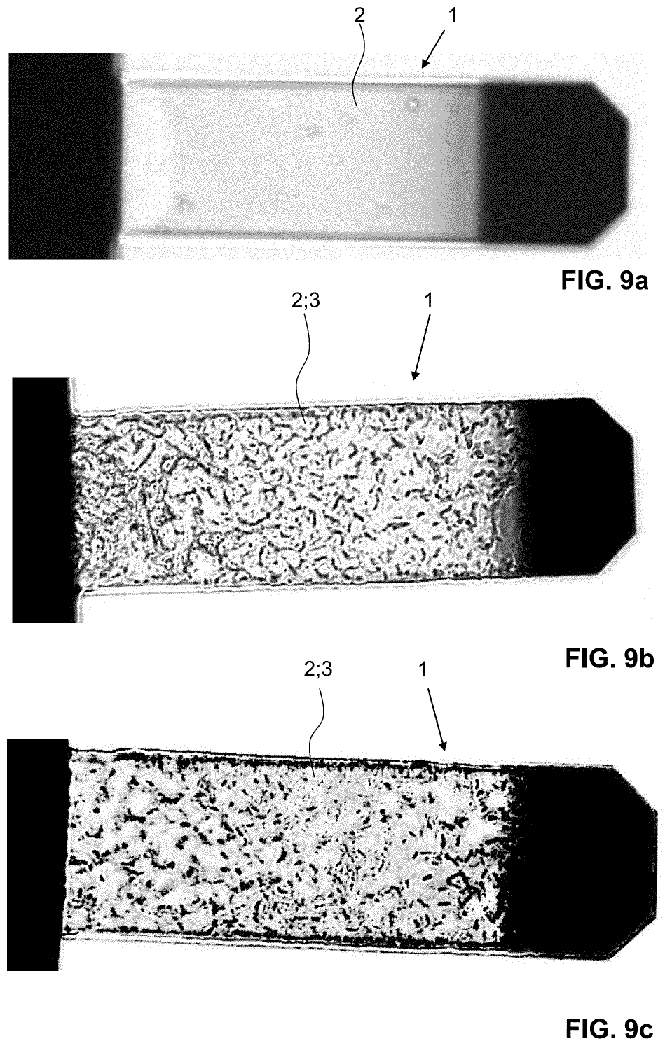

FIGS. 9a to 9c illustrate an attachment of biological objects on a substrate 1 in the form of a cantilever. The cantilever 1 of FIG. 9a has functionalized prior to the addition of the biological object with a solution comprising 0.5% glutaraldehyde by weight per total volume. However, no first compound was added to the suspension of the biological object E. coli resistant strain B1. The surface 2 of the cantilever 1 has been functionalized with a solution of 0.01% poly-D-lysine by weight prior to the addition of the biological object in the situations depicted in FIGS. 9b and 9c. Subsequently, a dispersion comprising a solution comprising 0.04% agar by weight per total volume of the solution and cells of the E. coli resistant strain B1 have been added to the functionalized surface 3 of the substrate 1 in a second step. FIG. 9c depicts a micrograph of the functionalized substrate 1 of FIG. 9b that has been recorded about 3 hours after the attachment of the cells to said substrate 1. As is readily evident from a comparison between FIGS. 9a to 9c, a much higher number of E. coli bacteria is attached on the cantilevers 1 according to the invention and as depicted in FIGS. 9b and 9c as compared to the cantilever 1 according to FIG. 9a whose surface 2 has functionalized but the cell suspension did not contain a first compound such as agar. Furthermore, FIG. 9c clearly shows that even after some time there is still a high number of attached cells present on the substrate 1. Hence, the invention allows a reliable attachment, wherein the cells remain attached to the surface during a period of time that is at least as long as a testing time.

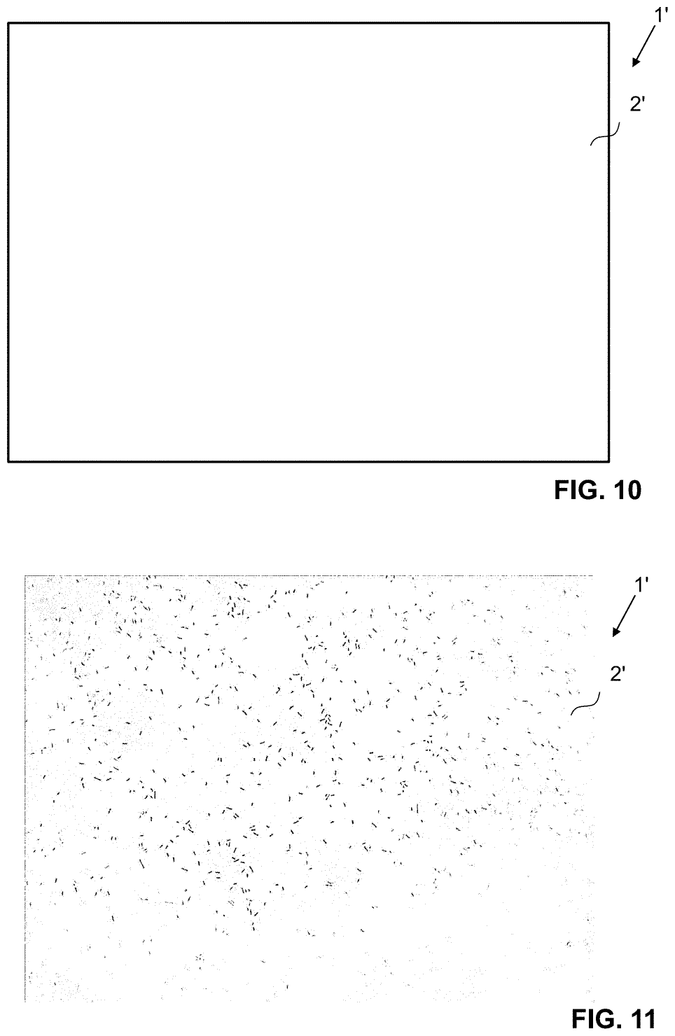

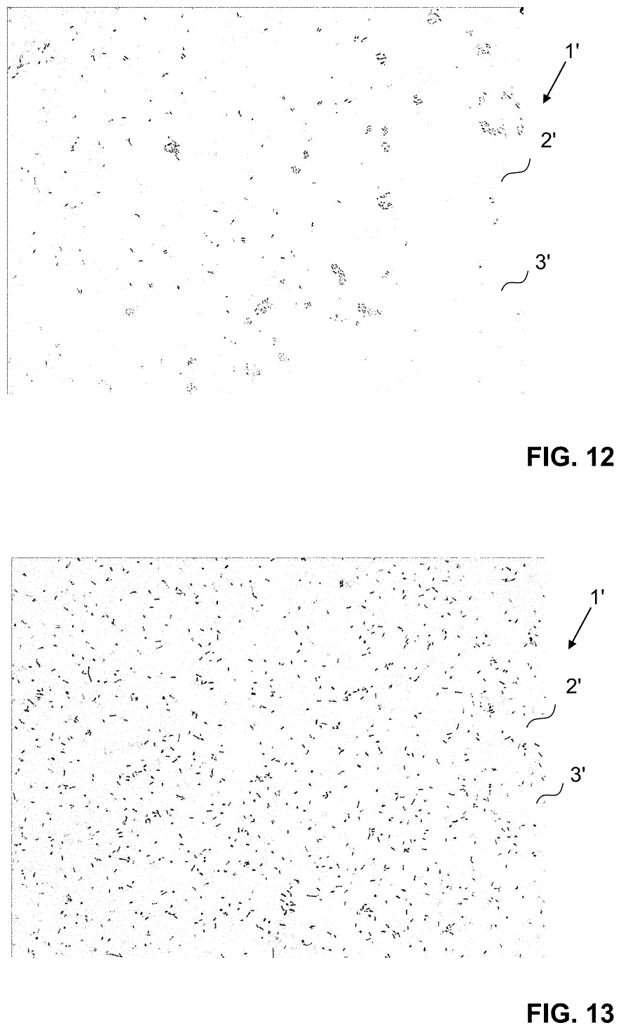

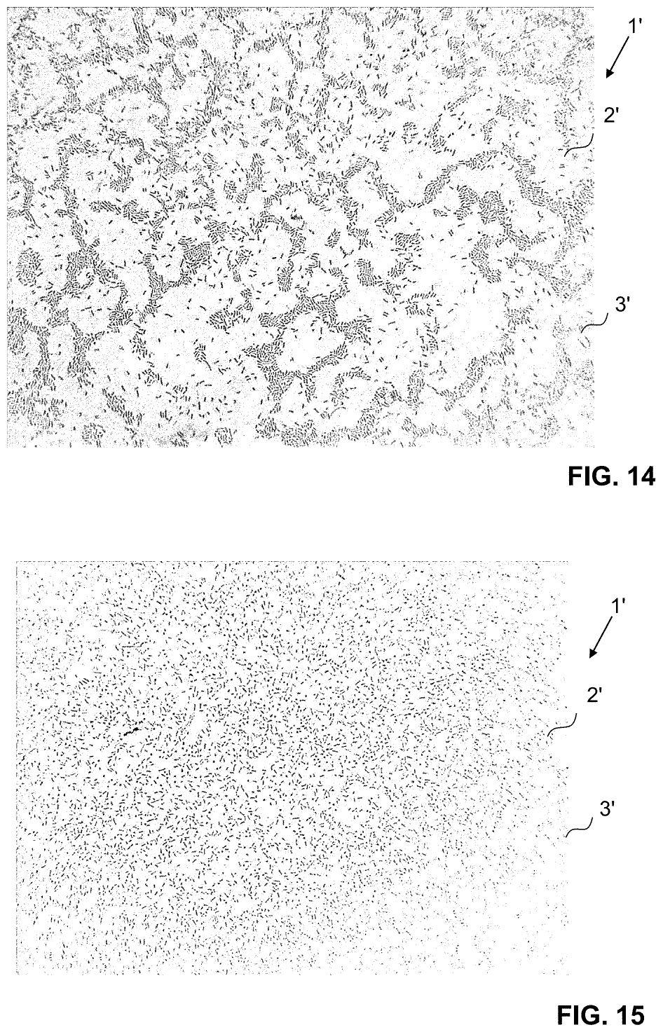

FIGS. 10 to 21 in each case depict an attachment of the E. coli ceftriaxone susceptible strain ATCC 25922 to differently treated substrates with different first compounds in the cell suspension. In particular, FIG. 10 shows a glass substrate that is a microscopic cover slide that has not been functionalized and where there has no first compound been added to the cell suspension. As can be seen, no cells of E. coli ceftriaxone susceptible strain ATCC 25922 attached to the surface after washing with water twice. The E. coli cells were grown overnight on Columbia agar plates as described above. The OD₆₀₀ of the cell suspension was 1.2 prior to the addition of the same to the surface. In FIG. 11, another untreated glass surface is shown that was treated with the same cells but this time the first compound was added to the cell suspension. The first compound was agar at a concentration 0.04% by weight. More cells adhered to the surface compared with FIG. 10. The FIGS. 12 and 13 show the same cells attached to another glass surface that has been functionalized using glutaraldehyde at a concentration of 0.5% by weight. No first compound was added to the cell suspension. FIG. 12 shows that cells adhered in small aggregates on the surface. The test was repeated with the same cells and another glass surface that was functionalized using 0.5% glutaraldehyde by weight but this time 0.04% agar by weight were added to the cell suspension. The result in FIG. 13 shows that a similar amount of cells had attached to the surface but they were evenly dispersed without cell aggregates. This test was repeated but this time, glutaraldehyde as second compound was replaced by 0.01% poly-D-lysine by weight to functionalize the surface. FIG. 14 shows that without agar more cells attached compared with FIG. 12 where the surface was functionalized using glutaraldehyde. The cells attached in larger aggregates compared with glutaraldehyde. As shown in FIG. 15, when 0.04% agar by weight was added to the surface, more cells attached compared to the test without agar. The attached cells were also more evenly distributed over the glass surface.

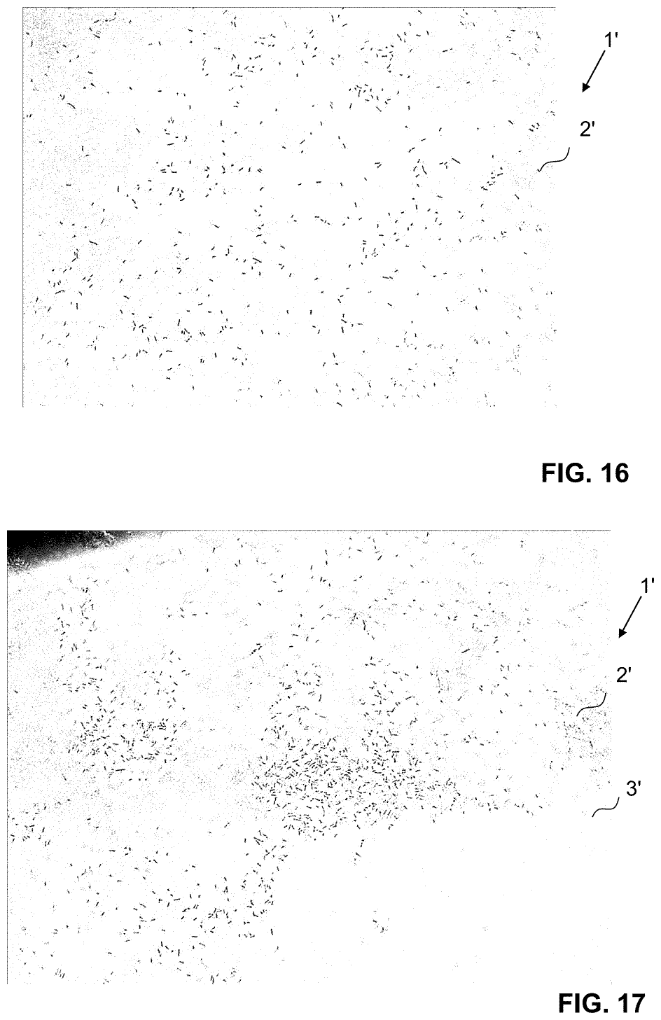

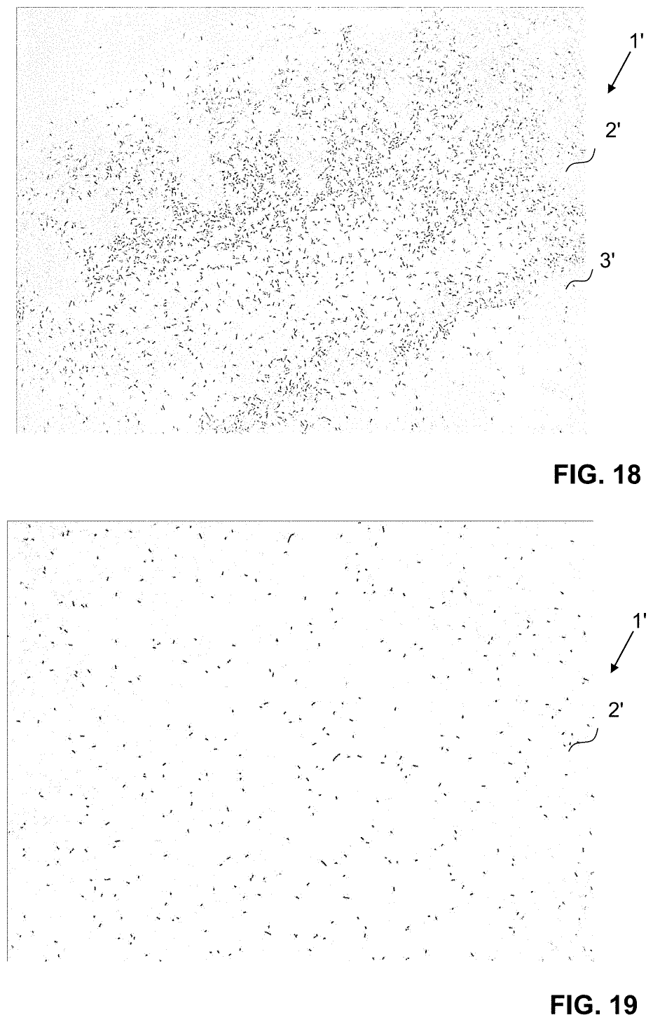

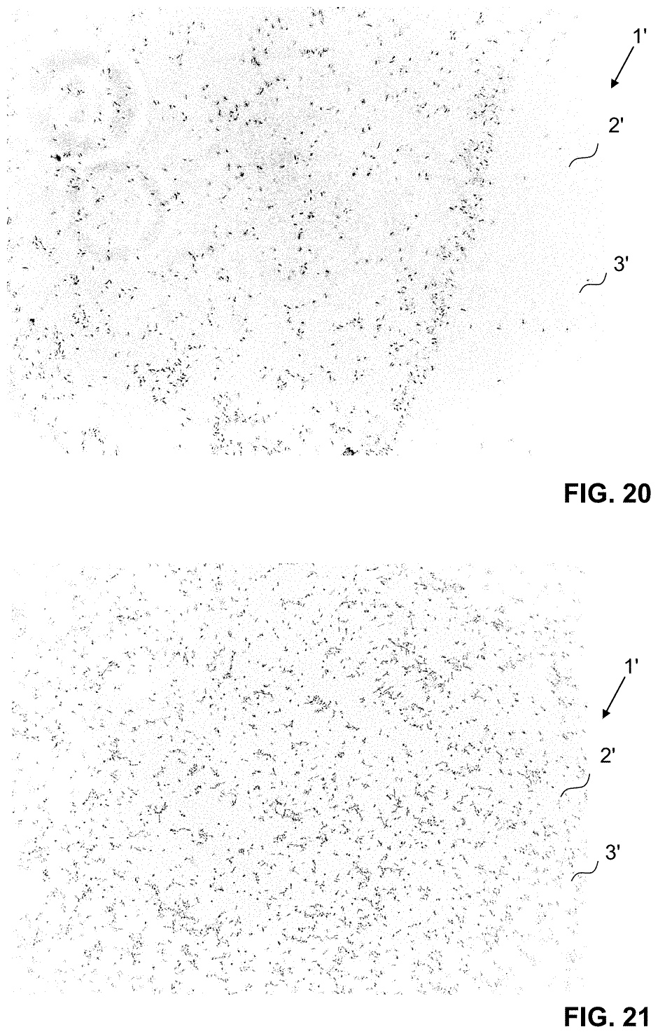

FIG. 16 depicts an attachment of the cells to the substrate, wherein 0.03% Nafion® as the first compound but no second compound were used. That is, the surface of the substrate was not functionalized but only a first dispersion comprising the cells and 0.03% Nafion® by weight was added to the substrate. FIG. 17 depicts an attachment of the cells to a substrate that has been treated with both, a first compound and a second compound. In particular, 0.5% glutaraldehyde as the second compound was used to functionalize the surface of the substrate in a first step. In a second step, a dispersion of the cells and 0.03% Nafion® by weight was added to the functionalized surface of the substrate. FIG. 18 depicts an attachment of the cells to the substrate that has likewise been treated with a first and a second compound. In this case, a solution of 0.1% chitosan by weight was used to functionalize the surface of the substrate. Thereafter, a dispersion comprising the cells and 0.03% Nafion® was added to the functionalized surface. A further improvement of cell attachment is apparent. FIG. 19 in turn depicts an attachment of the cells where no second compound has been used, i.e. the surface of the substrate has not been functionalized. Instead, a dispersion comprising the cells and 0.1% acrylamide was added to the unfunctionalized surface of the substrate. FIG. 20 depicts an attachment of cells to a functionalized surface. That is, the surface of the substrate has been functionalized by 0.5% glutaraldehyde, wherein subsequently the dispersion comprising the cells and 0.1% acrylamide by weight have been added to the functionalized surface. Similarly, FIG. 21 depicts an attachment of cells to a functionalized surface, wherein the surface has been functionalized with 0.1% chitosan by weight, to which a dispersion comprising the cells and 0.1% acrylamide by weight has been added. From these figures it is readily apparent that a cell attachment takes place if a first compound is added to the cell dispersion, and where no functionalization of the surface of the substrate has taken place, see FIGS. 16 and 19. Said attachment can however be further enhanced if the surface of the substrate is functionalized, see FIGS. 17 to 18 and 20 to 21.

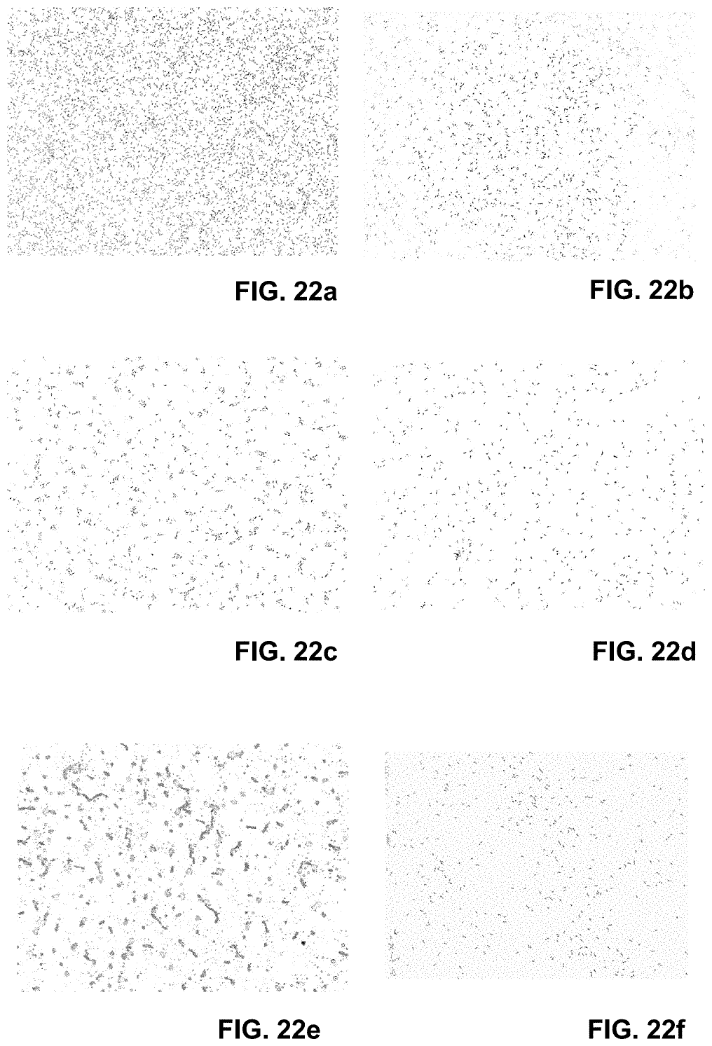

FIGS. 22a to 41g depict different images of a substrate in the form of a glass surface to which different objects have been added along with different first compounds and/or different second compounds.

In particular, FIGS. 22a to 22f depict the attachment of Enterobacteria Escherichia coli ATCC 25922 that were grown in a subculture for 1 hour at 37° C. and provided at a cell concentration OD₆₀₀=1 to hydrolytic class 1 glass, wherein the images were recorded after an attachment time of 5 minutes at 25° C. In all these figures the glass surface has been functionalized with a solution of 0.1 milligram per millilitre poly-D-lysine solution during 5 minutes at 25° C. in a first step. Thereafter, a dispersion comprising Escherichia coli ATCC 25922 and (a) 0.04% agarose by weight per total volume of the first solution (b) 0.04% agar by weight per total volume of the first solution, (c) 0.25% alginate by volume per total volume of the first solution, (d) 0.25% Nafion® by weight per total volume of the first solution, (e) 2% polydimethylsiloxane by volume per total volume of the first solution (stock solution at 20 Centistokes (cst)) resulting in 0.4 cst polydimethylsiloxane, and (f) 0.125% polyethylene glycol by weight per total volume of the first solution were added to the functionalized surface, respectively. Here and below, the expression weight per total volume of the first solution means “gram per 100 millilitre of solvent”, wherein water was used as the solvent. The expression volume per total volume of the first solution means “millilitre of a 100% stock solution per total volume of the first solution”, wherein the expression “100% stock solution” refers to a solution comprising 100% of the first compound. In other words, a “100% stock solution” is an undiluted solution of the first compound. It follows from these images that a good and homogeneous attachment of Escherichia coli ATCC 25922 was achieved.

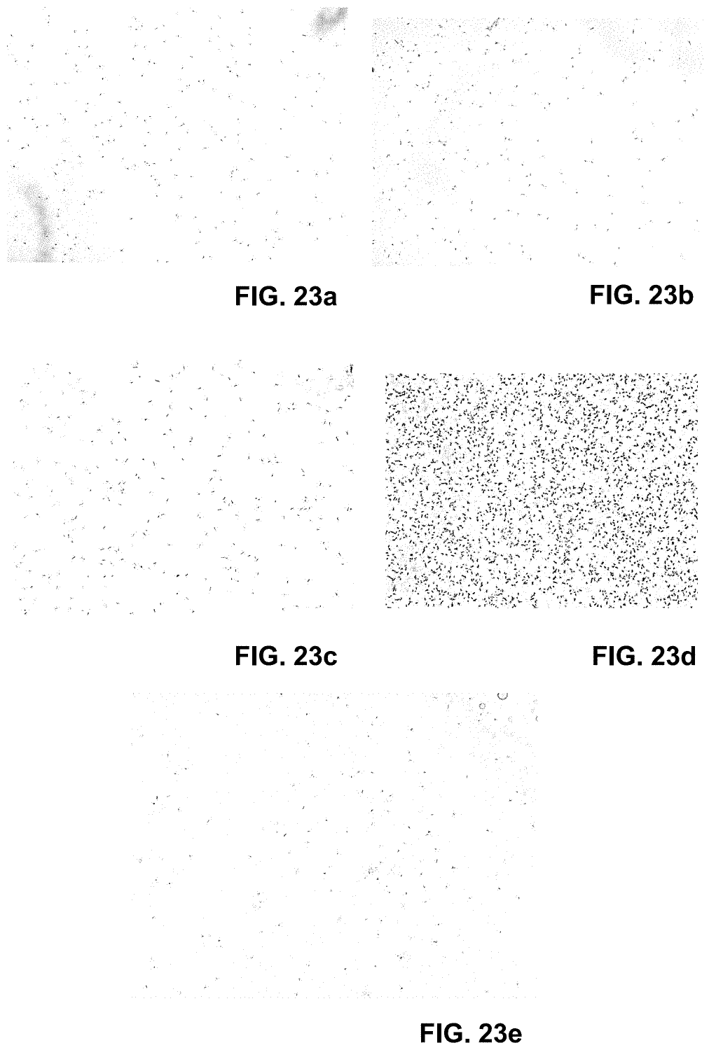

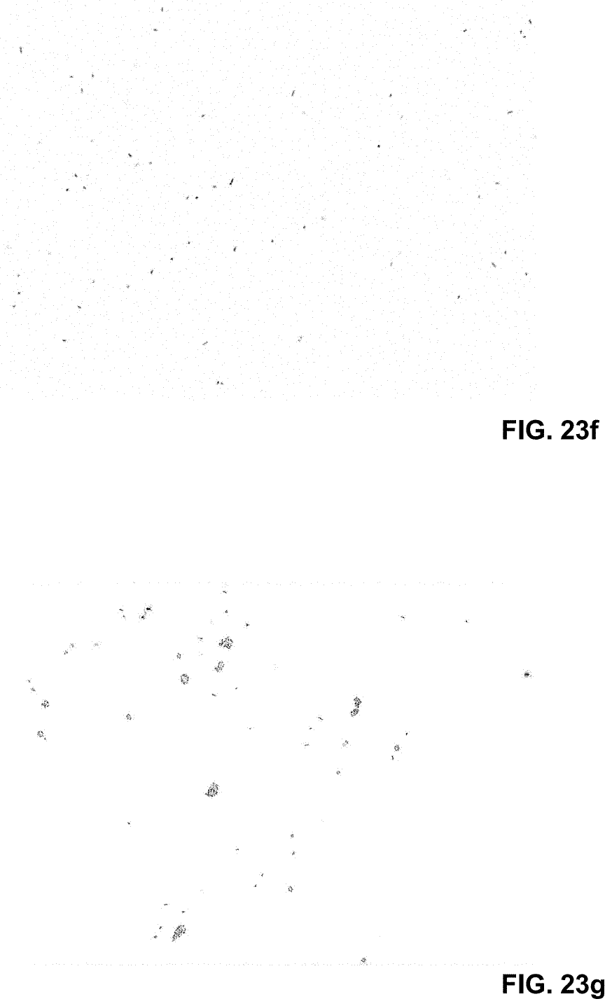

FIGS. 23a to 23g depict the attachment of Enterobacteria Escherichia coli ATCC 25922 that were grown in a subculture for 1 hour at 37° C. and provided at a cell concentration OD₆₀₀=1 to hydrolytic class 1 glass, wherein the images were recorded after an attachment time of 5 minutes at 25° C. In all these figures the glass surface has been functionalized with a solution of 5 microgram per square centimeter (μg/cm²) laminin solution during 60 minutes at 25° C. in a first step. Thereafter, a dispersion comprising Escherichia coli ATCC 25922 and (a) 0.04% agarose by weight per total volume of the first solution (b) 0.04% agar by weight per total volume of the first solution, (c) 0.25% alginate by volume per total volume of the first solution, (d) 0.25% Nafion® by weight per total volume of the first solution, (e) 0.4 cst polydimethylsiloxane, and (f) 0.125% polyethylene glycol by weight per total volume of the first solution were added to the functionalized surface, respectively. In (g) the attachment of a suspension of Escherichia coli ATCC 25922 in the absence of a first compound is depicted. It follows, that usage of a functionalized surface only resulted in only a few cells to be attached while several agglomerates are formed. The addition of a first compound to the cell suspension significantly increases the attachment quality and homogeneity, especially when Nafion® is used (FIG. 23d).

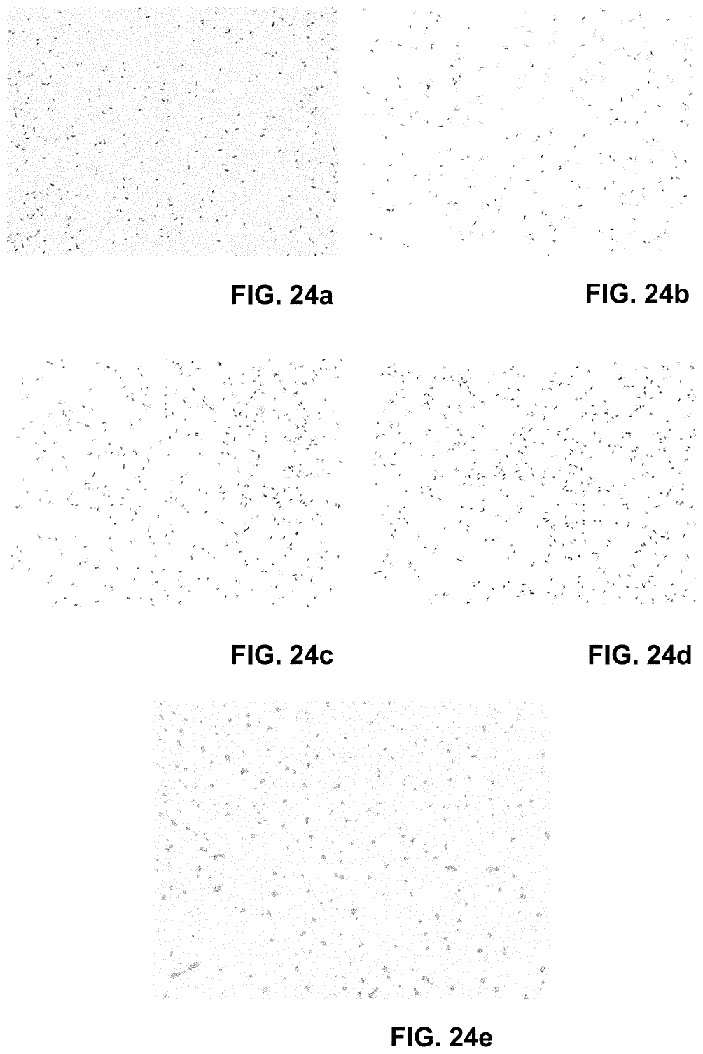

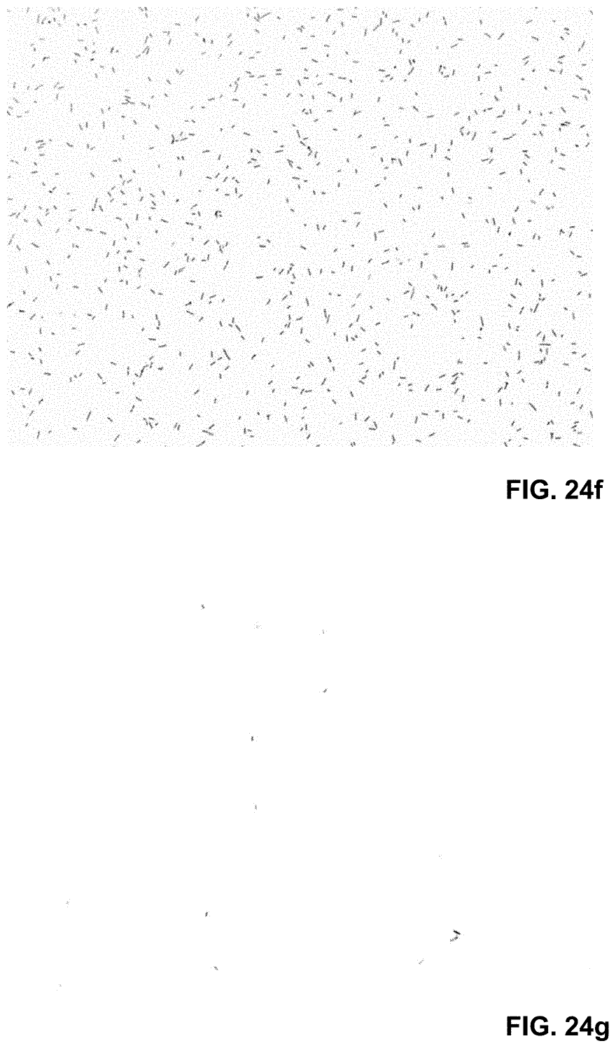

FIGS. 24a to 24g depict the attachment of Enterobacteria Escherichia coli ATCC 25922 that were grown in a subculture for 1 hour at 37° C. and provided at a cell concentration OD₆₀₀=1 to hydrolytic class 1 glass, wherein the images were recorded after an attachment time of 5 minutes at 25° C. In all these figures the glass surface has been functionalized with a solution of 0.1 milligram per millilitre chitosan solution during 5 minutes at 25° C. in a first step. Thereafter, a dispersion comprising Escherichia coli ATCC 25922 and (a) 0.04% agarose by weight per total volume of the first solution (b) 0.04% agar by weight per total volume of the first solution, (c) 0.25% alginate by volume per total volume of the first solution, (d) 0.25% Nafion® by weight per total volume of the first solution, (e) 0.4 cst polydimethylsiloxane, and (f) 0.125% polyethylene glycol by weight per total volume of the first solution were added to the functionalized surface, respectively. In (g) the attachment of a suspension of Escherichia coli ATCC 25922 in the absence of a first compound is depicted. It follows, that usage of a functionalized surface only resulted in only a few cells to be attached while several agglomerates are formed. As follows from these figures, if the cell suspension is added to the functionalized surface only and in the absence of a first compound, almost no cells are attached. The addition of a first compound drastically increased the number of cells as well as the quality, i.e. homogeneity of the attachment.

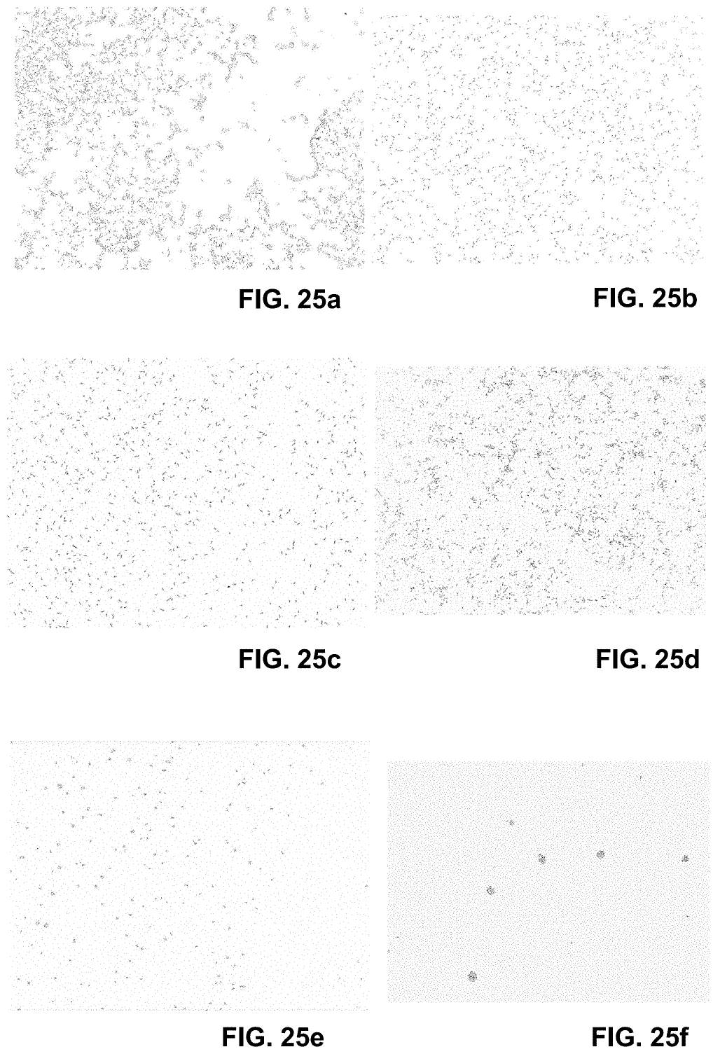

FIGS. 25a to 25f depict the attachment of Enterobacteria Escherichia coli ATCC 25922 that were grown in a subculture for 1 hour at 37° C. and provided at a cell concentration OD₆₀₀=1 to hydrolytic class 1 glass, wherein the images were recorded after an attachment time of 5 minutes at 25° C. In all these figures the glass surface has been functionalized with a solution of 0.5% glutaraldehyde by volume per total volume of said solution in a first step. Thereafter, a dispersion comprising Escherichia coli ATCC 25922 and (a) 0.04% agarose by weight per total volume of the first solution (b) 0.04% agar by weight per total volume of the first solution, (c) 0.25% alginate by volume per total volume of the first solution, (d) 0.25% Nafion® by weight per total volume of the first solution, (e) 0.4 cst polydimethylsiloxane, and (f) 0.125% polyethylene glycol by weight per total volume of the first solution were added to the functionalized surface, respectively. As follows from these figures the addition of a first compound to the cell suspension reduces the presence of agglomerates and increases the numbers of cells attached.

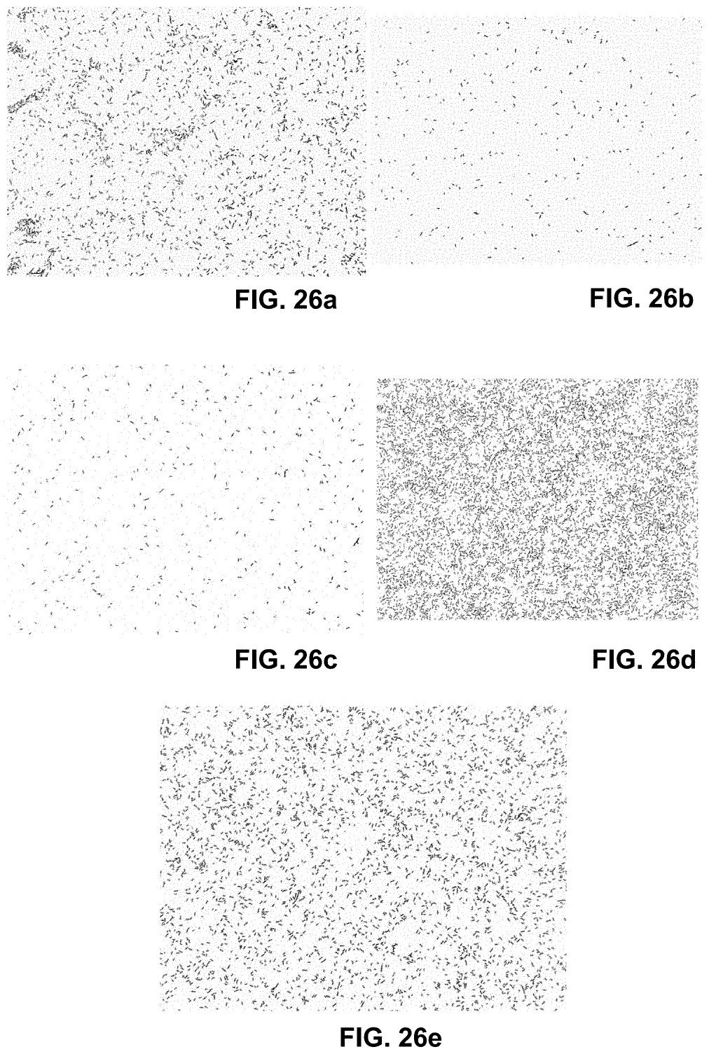

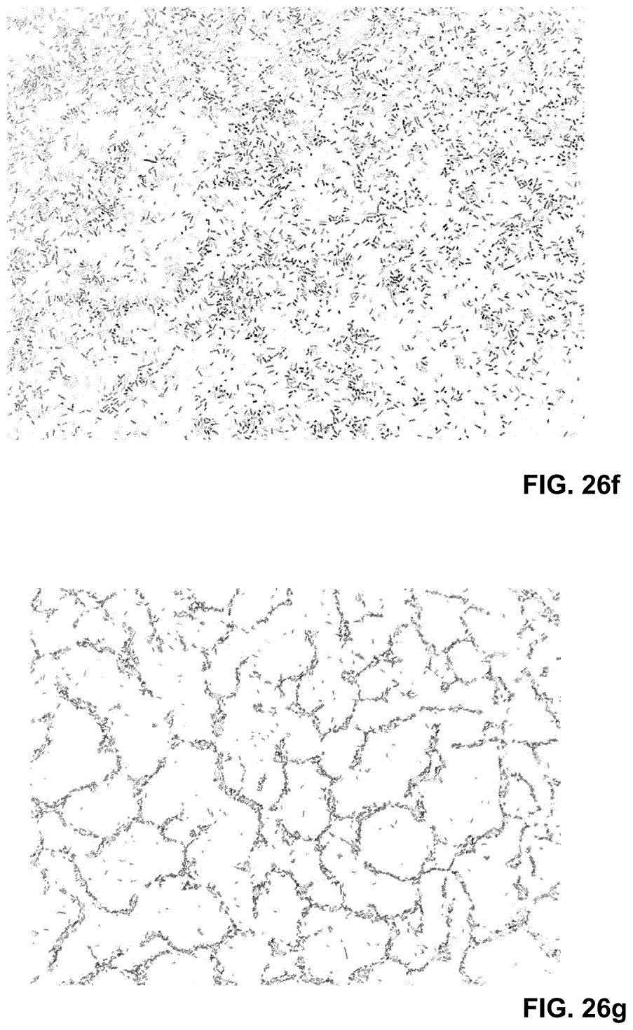

FIGS. 26a to 26g depict the attachment of Enterobacteria Escherichia coli ATCC 25922 that were grown in a subculture for 1 hour at 37° C. and provided at a cell concentration OD₆₀₀=1 to hydrolytic class 1 glass, wherein the images were recorded after an attachment time of 5 minutes at 25° C. In all these figures the glass surface has been functionalized with a solution of (3-aminopropyl)triethoxysilane of 1% by volume per total volume of said solution during 5 minutes at 25° C. in a first step. Thereafter, a dispersion comprising Escherichia coli ATCC 25922 and (a) 0.04% agarose by weight per total volume of the first solution (b) 0.04% agar by weight per total volume of the first solution, (c) 0.25% alginate by volume per total volume of the first solution, (d) 0.25% Nafion® by weight per total volume of the first solution, (e) 0.4 cst polydimethylsiloxane, and (f) 0.125% polyethylene glycol by weight per total volume of the first solution were added to the functionalized surface, respectively. In (g) the attachment of a suspension of Escherichia coli ATCC 25922 in the absence of a first compound is depicted. It follows that usage of 3-aminopropyl)triethoxysilane alone results in highly agglomerated cells and an unhomogeneous attachment. The addition of a first compound to the cell suspension significantly reduces the generation of agglomerates and the number of attached cells was increased.

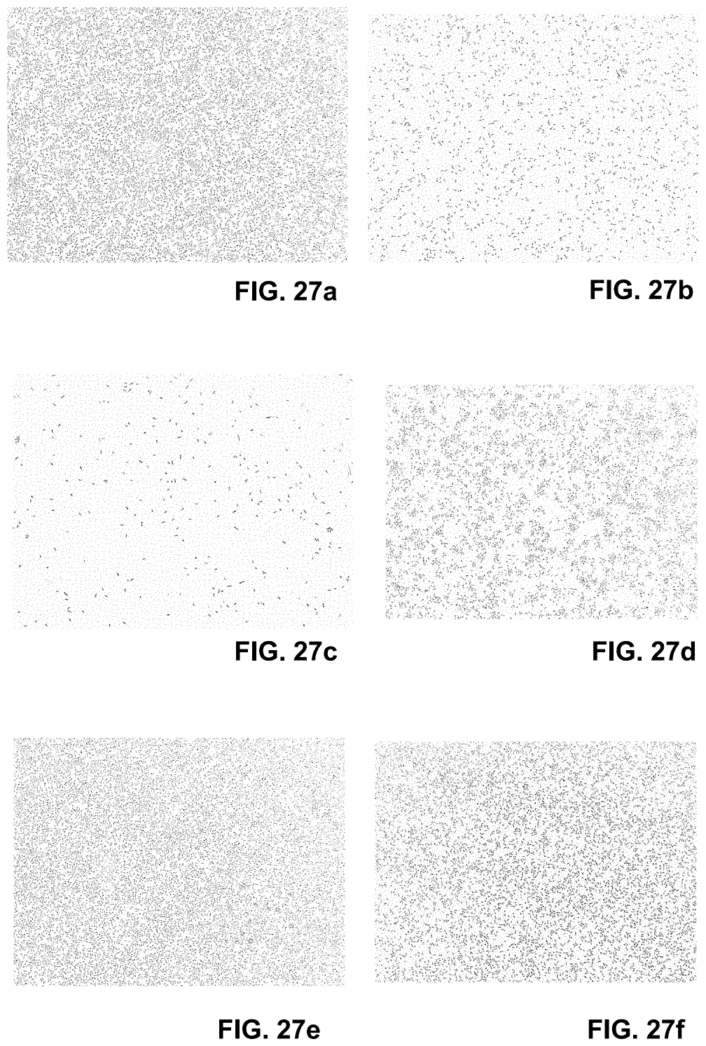

FIGS. 27a to 27f depict the attachment of Enterobacteria Escherichia coli ATCC 25922 that were grown in a subculture for 1 hour at 37° C. and provided at a cell concentration OD₆₀₀=1 to hydrolytic class 1 glass, wherein the images were recorded after an attachment time of 5 minutes at 25° C. In all these figures the glass surface has been functionalized first with a solution comprising 1 milligram per millilitre of poly(sodium-p-styrene sulfonate) and then by a 1 milligram per millilitre solution of poly(allylamine hydrochloride to finally form a co-polymer (i.e. a PAH/PSS co-polymer) during 20 minutes at 25° C. for each solution. The PAH/PSS co-polymer is a so-called layer-by-layer polymer, wherein the co-polymer is formed by incubating one compound at a time. In the present case the PSS polymer was incubated in a first step and the PAH polymer was incubated in a subsequent second step. The formation of this layer-by-layer polymer as a surface coating of the glass was performed initially. Thereafter, a dispersion comprising Escherichia coli ATCC 25922 and (a) 0.04% agarose by weight per total volume of the first solution (b) 0.04% agar by weight per total volume of the first solution, (c) 0.25% alginate by volume per total volume of the first solution, (d) 0.25% Nafion® by weight per total volume of the first solution, (e) 0.4 cst polydimethylsiloxane, and (f) 0.125% polyethylene glycol by weight per total volume of the first solution were added to the functionalized surface, respectively. As follows from these figures the addition of a first compound to the cell suspension reduces the presence of agglomerates and increases the numbers of cells attached. Also from these figures, it follows that a high number of cells are attached homogeneously when a first compound is added to the cell suspension.

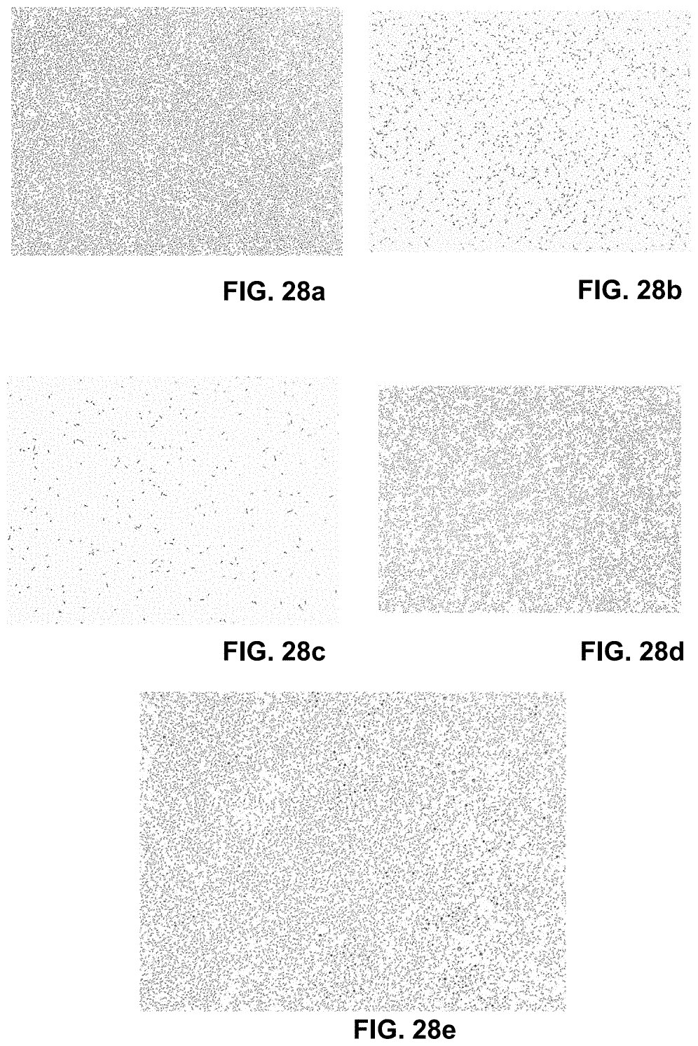

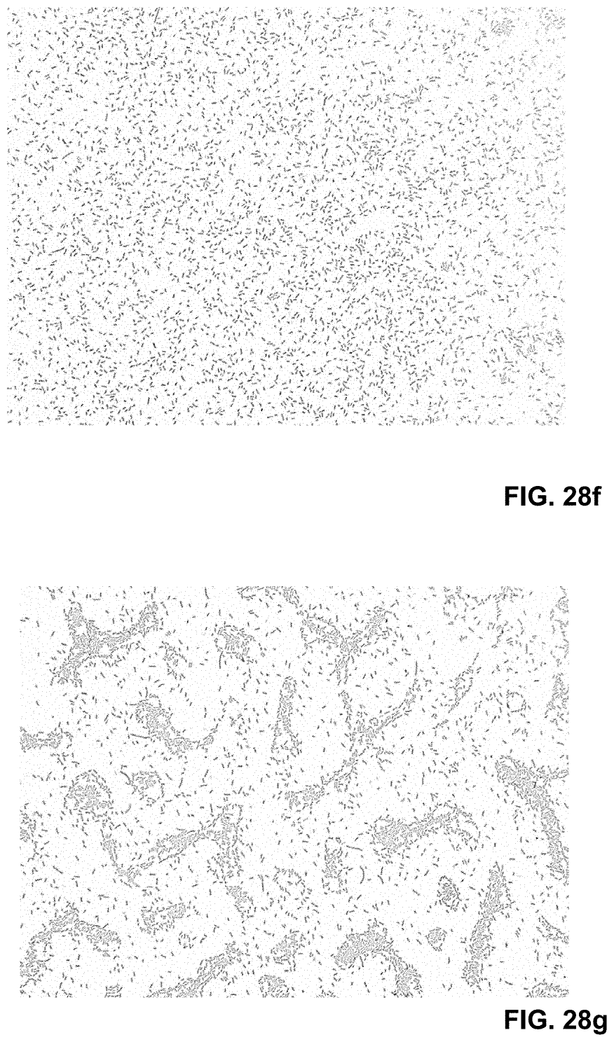

FIGS. 28a to 28g depict the attachment of Enterobacteria Escherichia coli ATCC 25922 that were grown in a subculture for 1 hour at 37° C. and provided at a cell concentration OD₆₀₀=1 to hydrolytic class 1 glass, wherein the images were recorded after an attachment time of 5 minutes at 25° C. In all these figures the glass surface has been functionalized with a solution of 1 milligram per millilitre Mussel Adhesive recombinant protein (MAPTrix™) solution during 30 minutes at 25° C. in a first step. Thereafter, a dispersion comprising Escherichia coli ATCC 25922 and (a) 0.04% agarose by weight per total volume of the first solution (b) 0.04% agar by weight per total volume of the first solution, (c) 0.25% alginate y volume per total volume of the first solution, (d) 0.25% Nafion® by weight per total volume of the first solution, (e) 0.4 cst polydimethylsiloxane, and (f) 0.125% polyethylene glycol by weight per total volume of the first solution were added to the functionalized surface, respectively. In (g) the attachment of a suspension of Escherichia coli ATCC 25922 in the absence of a first compound is depicted. As can be seen from these figures MAPTrix™ alone attaches the cells with a high amount of agglomerates. The tested first compounds resulted in an increased number of cells attached in a very homogeneous manner.

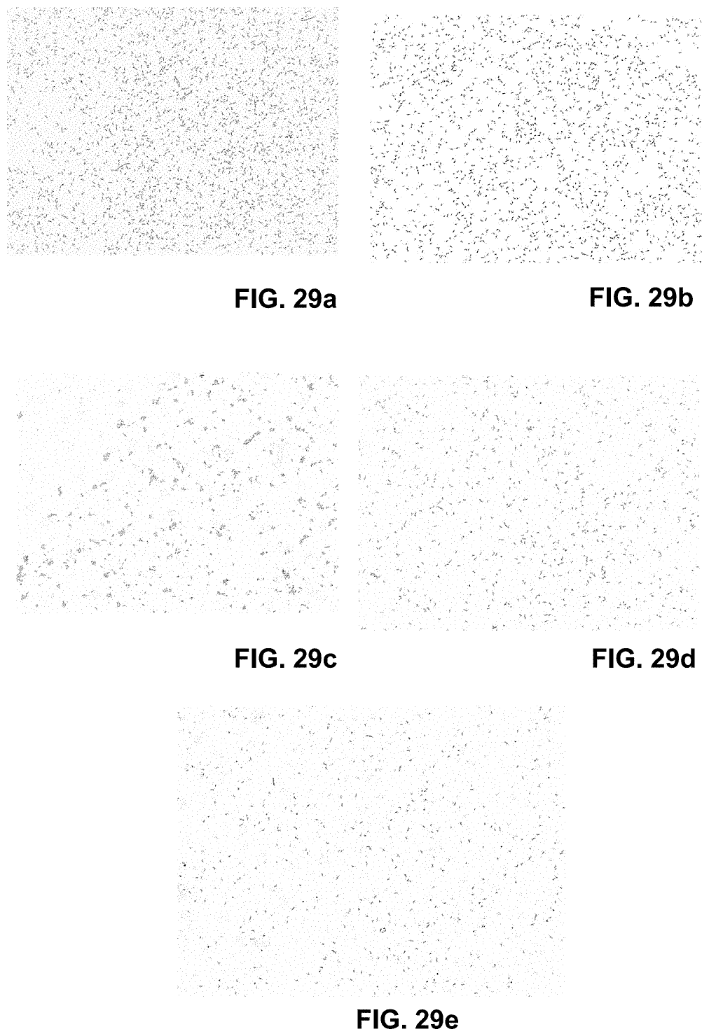

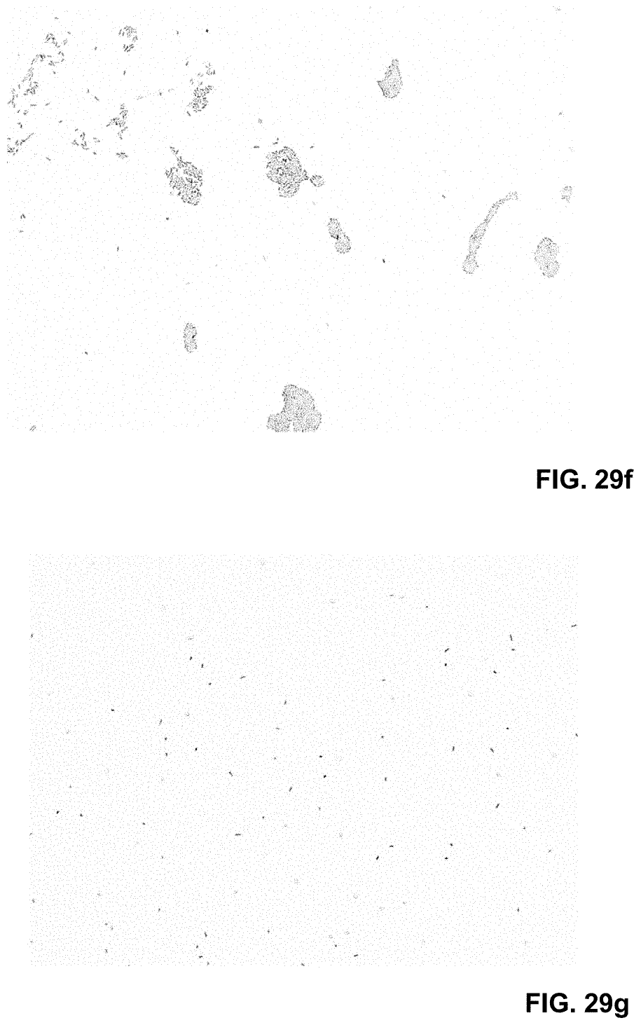

FIGS. 29a to 29g depict the attachment of Enterobacteria Escherichia coli ATCC 25922 that were grown in a subculture for 1 hour at 37° C. and provided at a cell concentration OD₆₀₀=1 to hydrolytic class 1 glass, wherein the images were recorded after an attachment time of 5 minutes at 25° C. In all these figures the glass surface has been functionalized with a 10 mM solution of 4-aminothiophenol during 20 minutes at 25° C. in a first step. Thereafter, a dispersion comprising Escherichia coli ATCC 25922 and (a) 0.04% agarose by weight per total volume of the first solution (b) 0.04% agar by weight per total volume of the first solution, (c) 0.25% alginate by volume per total volume of the first solution, (d) 0.25% Nafion® by weight per total volume of the first solution, (e) 0.4 cst polydimethylsiloxane, and (f) 0.125% polyethylene glycol by weight per total volume of the first solution were added to the functionalized surface, respectively. In (g) the attachment of a suspension of Escherichia coli ATCC 25922 in the absence of a first compound is depicted. As can be seen from these FIGS. 4-aminothiophenol resulted in almost no cells being attached. Usage of a first compound significantly increased the attachment quality.

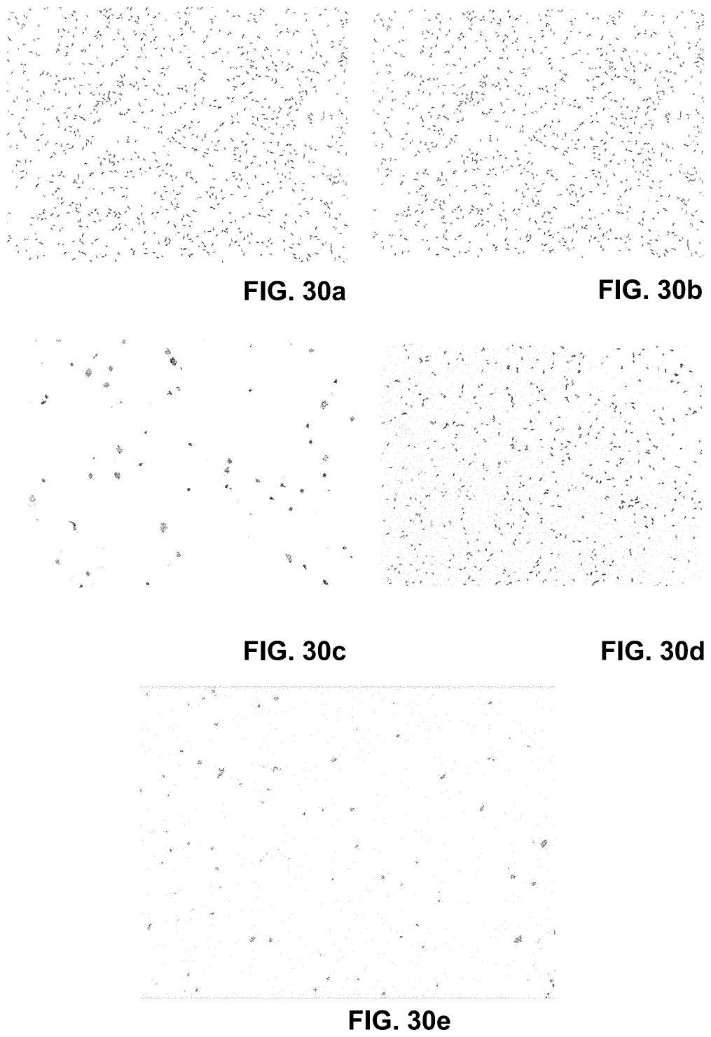

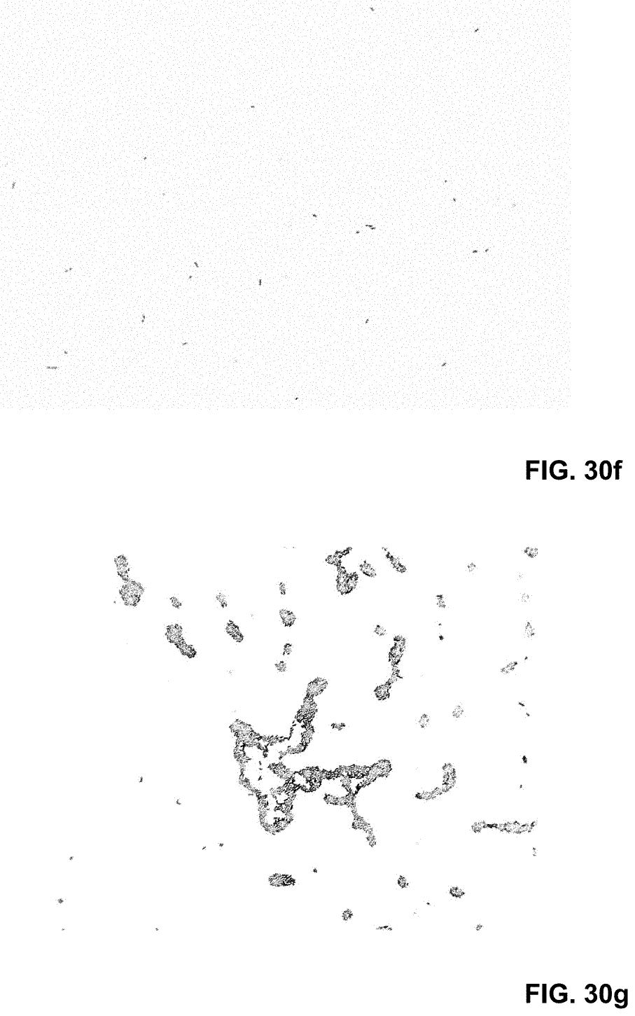

FIGS. 30a to 30g depict the attachment of Enterobacteria Escherichia coli ATCC 25922 that were grown in a subculture for 1 hour at 37° C. and provided at a cell concentration OD₆₀₀=1 to an unfunctionalized hydrolytic class 1 glass. In FIGS. 30a to 30f the cell dispersion comprising the bacteria and a first compound was added to the unfunctionalized substrate. The first compounds were as follows: (a) 0.04% agarose by weight per total volume of the first solution (b) 0.04% agar by weight per total volume of the first solution, (c) 0.25% alginate by volume per total volume of the first solution, (d) 0.25% Nafion® by weight per total volume of the first solution, (e) 0.4 cst polydimethylsiloxane, and (f) 0.125% polyethylene glycol by weight per total volume of the first solution. FIG. 30g depicts an image of an untreated substrate to which the said cell suspension in the absence of any first compound has been added. It follows that the absence of a functionalized surface as well as the absence of a first compound result in the formation of compact cell agglomerates. The presence of a first compound results in a homogeneous attachment of many cells.

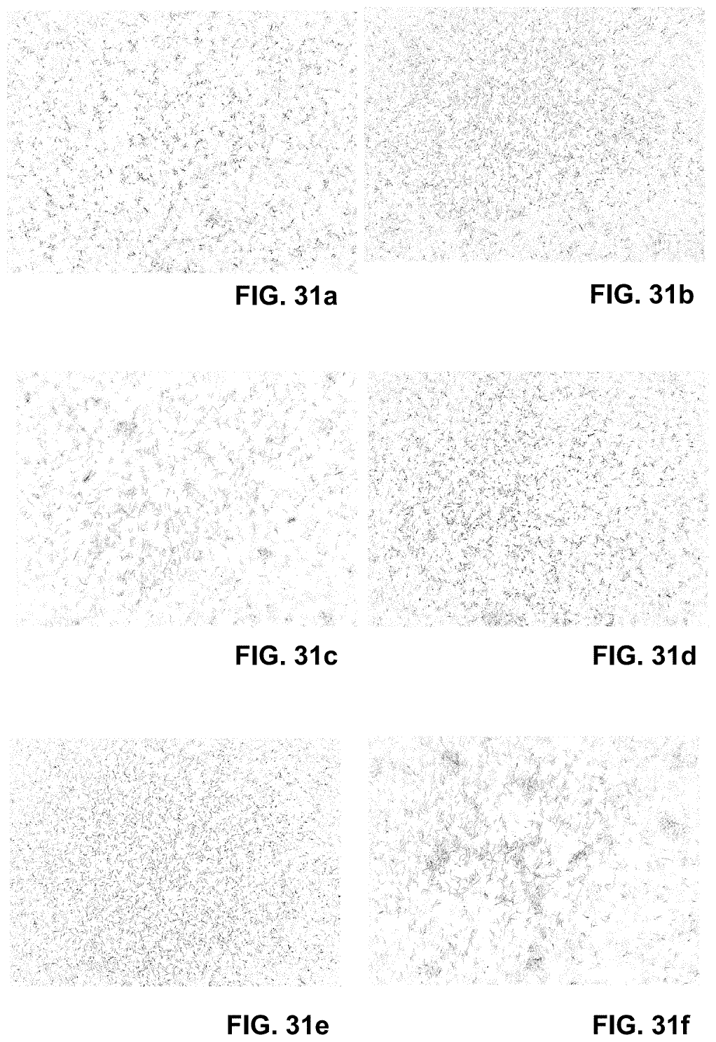

FIGS. 31a to 31f depict the attachment of Mycobacteria Mycobacterium smegmatis MC(2)155 that were grown in a subculture for 1 hour at 37° C. and provided at a cell concentration OD₆₀₀=1 to hydrolytic class 1 glass, wherein the images were recorded after an attachment time of 5 minutes at 25° C. In FIGS. 31a to 31c the glass surface has been functionalized with a solution comprising 0.1 milligram per millilitre poly-D-lysine during 5 minutes at 25° C. in a first step. Furthermore, FIGS. 31a and 31b depict the substrate after the addition of a dispersion comprising Mycobacterium smegmatis MC(2)155 and (a) 0.04% agar by weight per total volume of the first solution and (b) 0.04% agarose by weight per total volume of the first solution, respectively. FIG. 31c depicts the addition of a cell suspension in the absence of a first compound. The presence of the first compound increase the numbers of cells attached. FIGS. 31d and 31e depict an untreated substrate, wherein the cell dispersion comprising the cells and the first compound have been added to the unfunctionalized glass substrate. To this end FIG. 31d depicts the attachment of a dispersion comprising 0.04 agar by weight per total volume of the first solution and FIG. 31e depicts the attachment of a dispersion comprising 0.04% agarose by weight per total volume of the first solution. FIG. 31f depicts an untreated, i.e. unfunctionalized substrate wherein the cell suspension per se, i.e. in the absence of a first compound has been added to the substrate. It follows that the presence of a first compound decreases the presence of agglomerates and increases the number of cells being attached.

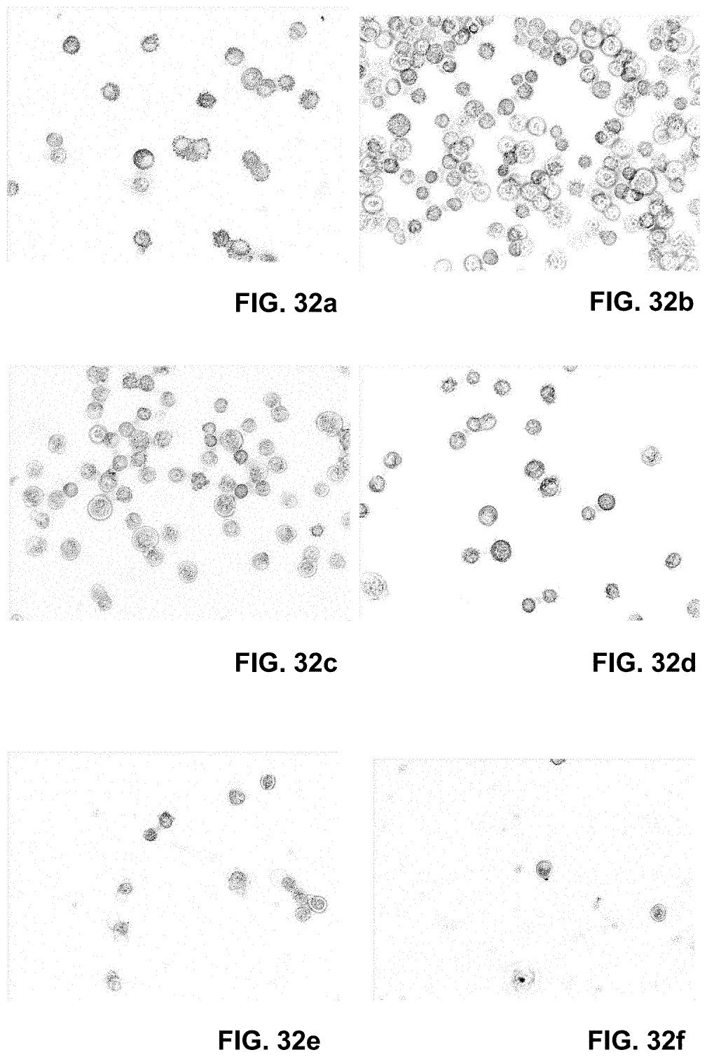

FIGS. 32a to 32f depict the attachment of Mammalian cells, here of Vero cells of the strain ATCC CCL-81. It should be noted that the cellular shape of the depicted attached cells does not correspond to the real shape of Vero cells. This is due to the trypsinization of the cells, necessary to detach them from their initial culture (Vero cells are so-called adherent cells). This process results in detachment of the cells which, without their substrate and sister cells (Vero cells naturally grow to form a confluent cell monolayer), in a loss of shape characterized by a circularization of the cells. Detached cells can be counted and diluted to the desired concentration. This step is therefore necessary to work with such cells. In the present case, as the images were recorded only a few minutes after attachment, the cells did not have the time to reacquire their natural shape. It usually takes them several hours to recover totally from trypsinization. However, despite their unnatural shape the cells still behave in their natural manner such that the present images can be seen as an adequate proof of attachment of Vero cells. To this end, FIGS. 32a to 32f depict the attachment of Vero cells of the strain ATCC CCL-81 that were grown in a subculture for 1 hour at 37° C. and provided at a cell concentration of 3.35×10³ cells per millilitre to hydrolytic class 1 glass, wherein the images were recorded after an attachment time of 5 minutes at 25° C. In FIGS. 32a and 32b the glass surface has been functionalized with a solution comprising 0.1 milligram per millilitre poly-D-lysine during 5 minutes at 25° C. in a first step. Furthermore, FIGS. 32a and 32b depict the substrate after the addition of a dispersion comprising Vero cells ATCC CCL-81 and (a) 0.04% agar by weight per total volume of the first solution and (b) 0.04% agarose by weight per total volume of the first solution, respectively. FIG. 32c depicts the addition of a cell suspension in the absence of a first compound. The presence of the first compound increases the numbers of cells attached, especially in the case of agarose. FIGS. 32d and 32e depict an untreated substrate, wherein the cell dispersion comprising the cells and the first compound have been added to the unfunctionalized glass substrate. To this end FIG. 32d depicts the attachment of a dispersion comprising 0.04 agar by weight per total volume of the first solution and FIG. 32e depicts the attachment of a dispersion comprising 0.04% agarose by weight per total volume of the first solution. FIG. 32f depicts an untreated, i.e. unfunctionalized substrate wherein the cell suspension per se, i.e. in the absence of a first compound has been added to the untreated substrate. It follows that without a second compound for the surface treatment and a first compound as additive to the cell suspension only a few cells are able to attach (Vero cells are adherent cells and therefore they have the innate capacity to attach to surfaces with time. The addition of Agarose alone resulted in slightly more cells being attached. Agar on the other hand resulted in more cells attached. It is assumed that this difference is due to the difference of the polysaccharide mesh strength (i.e. Agar is more “dense” than Agarose for a same concentration).

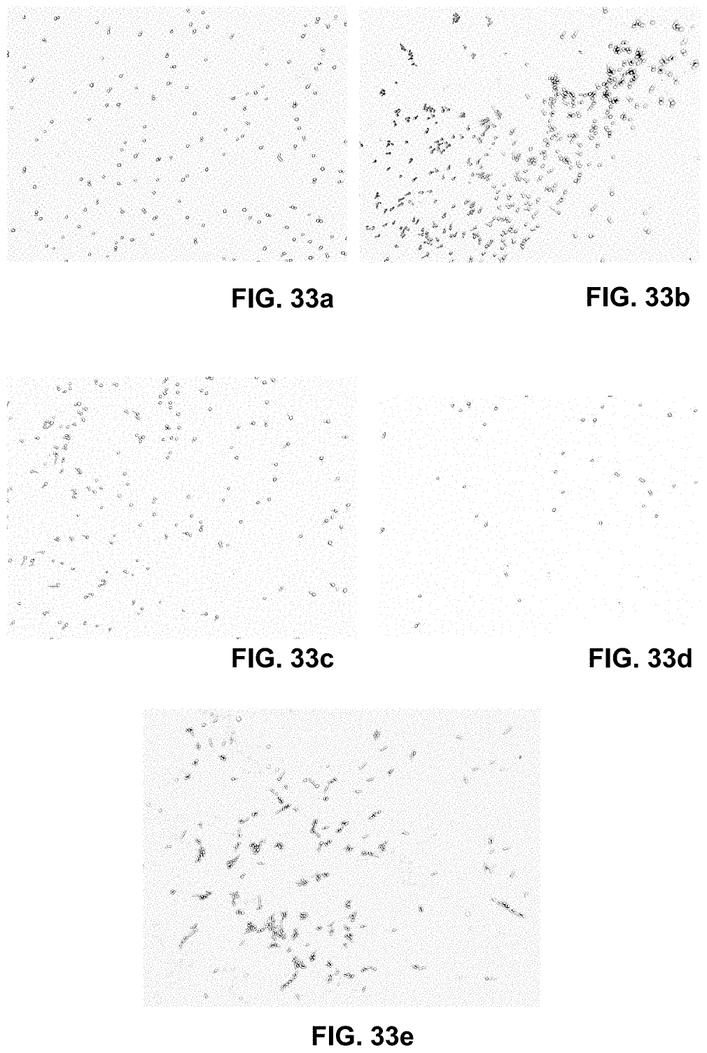

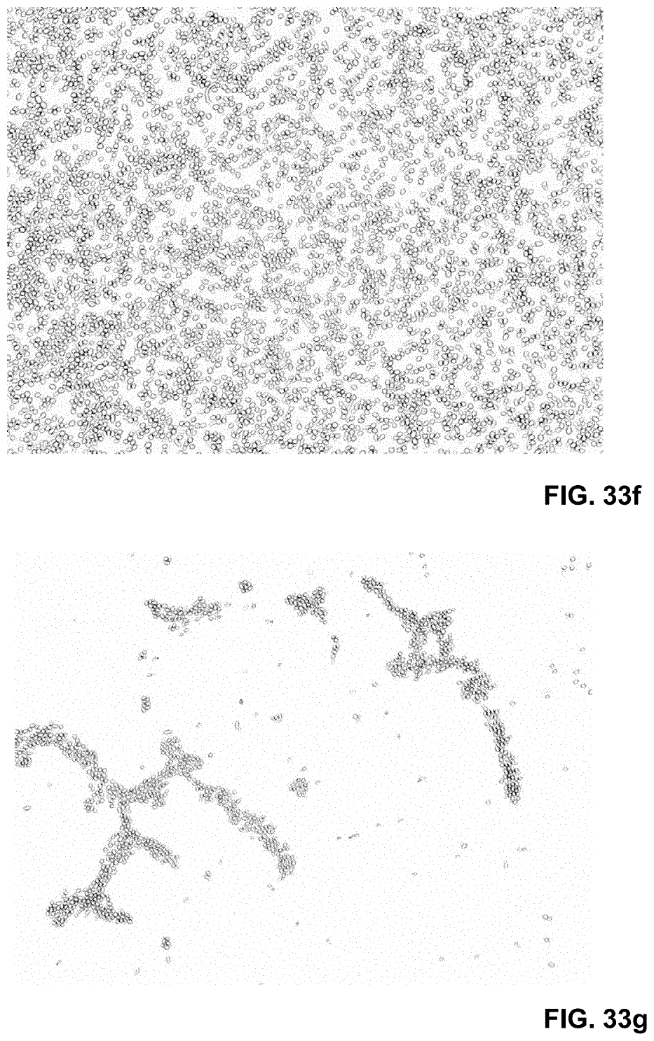

FIGS. 33a to 41g depict the attachment of yeast Candida albicans SC5314 that were grown in a subculture for 1 hour at 37° C. and provided at a cell concentration of OD₆₀₀=1 to hydrolytic class 1 glass, wherein the images were recorded after an attachment time of 5 minutes at 25° C.

In FIGS. 33a to 33g the glass surface has been functionalized with a solution of 0.1 milligram per millilitre poly-D-lysine solution during 5 minutes at 25° C. in a first step. Thereafter, a dispersion comprising Candida albicans SC5314 and (a) 0.04% agarose by weight per total volume of the first solution (b) 0.04% agar by weight per total volume of the first solution (c) 0.25% alginate by volume per total volume of the first solution, (d) 0.25% Nafion® by weight per total volume of the first solution, (e) 0.4 cst polydimethylsiloxane, and (f) 0.125% polyethylene glycol by weight per total volume of the first solution were added to the functionalized surface, respectively. FIG. 33g depicts the attachment of a suspension of Candida albicans SC5314 in the absence of a first compound. It follows that the presence of a functionalized surface alone, i.e. an addition of a cell suspension in the absence of a first compound, results in the presence of large agglomerates. The addition of a first compounds reduces the presence of agglomerates significantly. Furthermore, the number of cells being attached is increased.

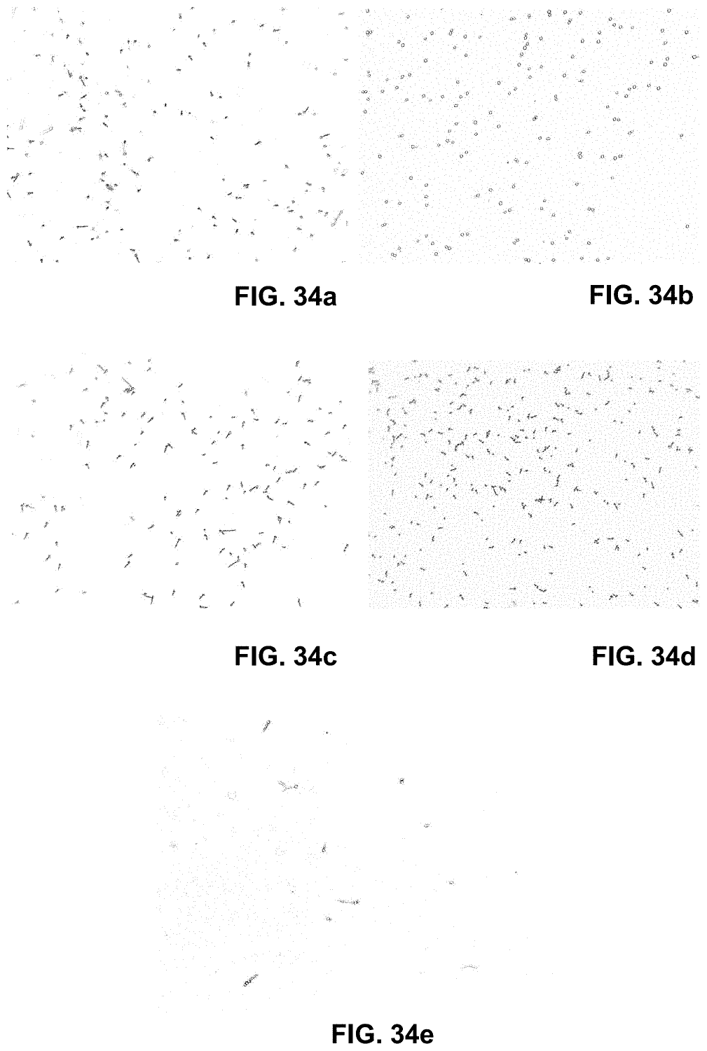

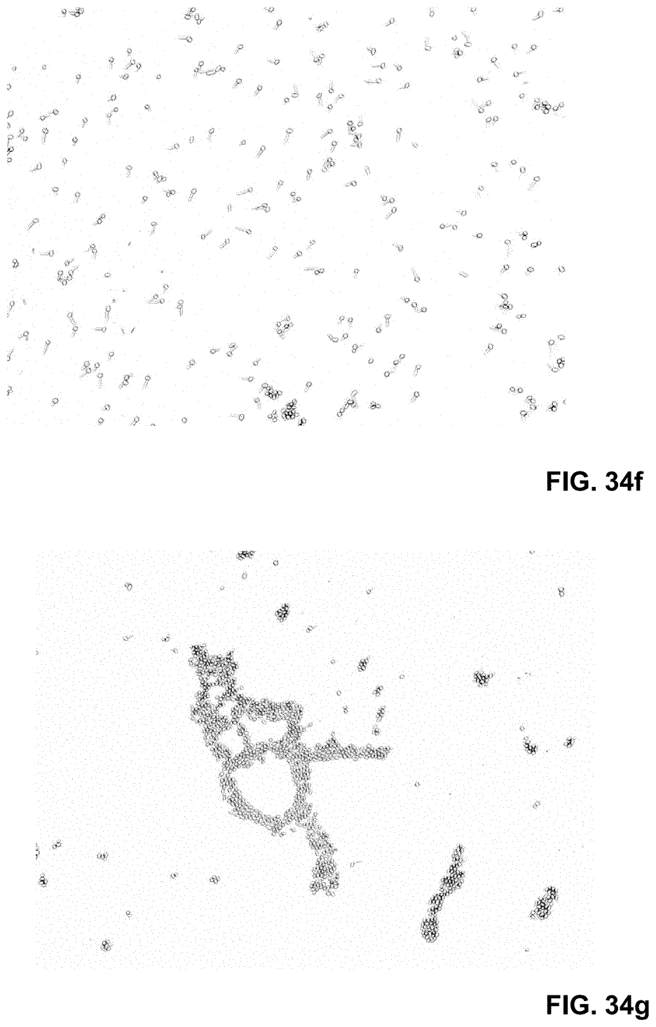

In FIGS. 34a to 34g the glass surface has been functionalized with a solution of 5 microgram per square centimeter (μg/cm²) laminin solution during 5 minutes at 25° C. in a first step. Thereafter, a dispersion comprising Candida albicans SC5314 and (a) 0.04% agarose by weight per total volume of the first solution (b) 0.04% agar by weight per total volume of the first solution, (c) 0.25% alginate by volume per total volume of the first solution, (d) 0.25% Nafion® by weight per total volume of the first solution, (e) 0.4 cst polydimethylsiloxane, and (f) 0.125% polyethylene glycol by weight per total volume of the first solution were added to the functionalized surface, respectively. In (g) the attachment of a suspension of Candida albicans SC5314 in the absence of a first compound is depicted. It follows that an attachment in the absence of a first compound in the cell suspension resulted in an attachment that is not homogeneous throughout the substrate but with some zones showing a very dense population and some zones showing only a few agglomerates. The addition of a first compound homogenizes the attachment throughout the substrate, while reducing the presence of agglomerates.

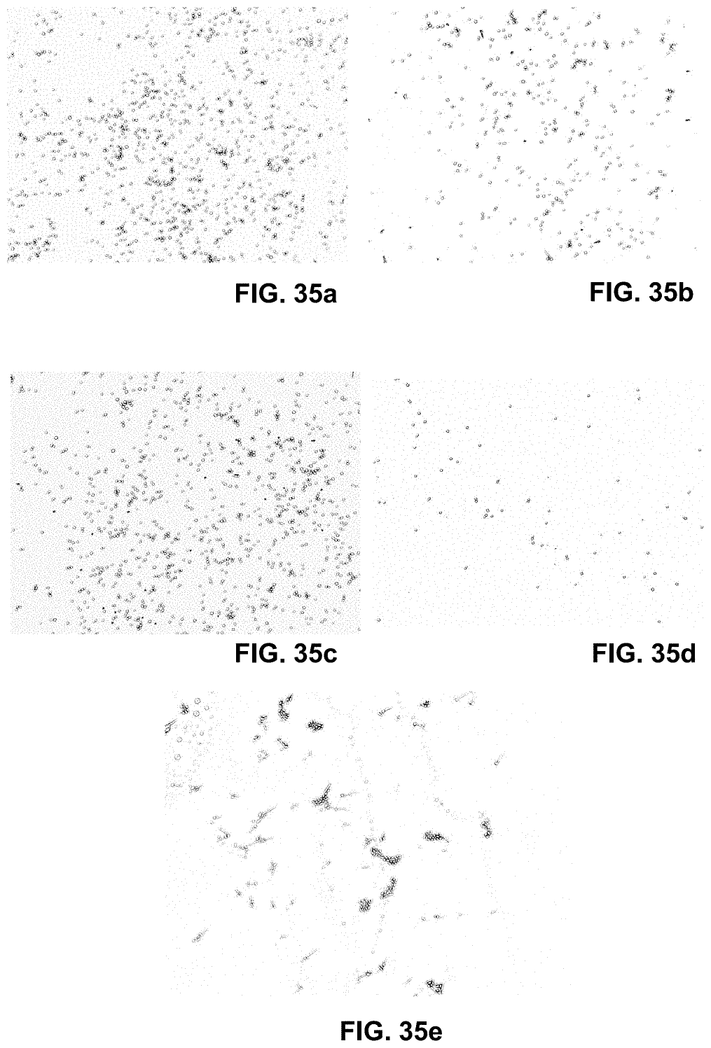

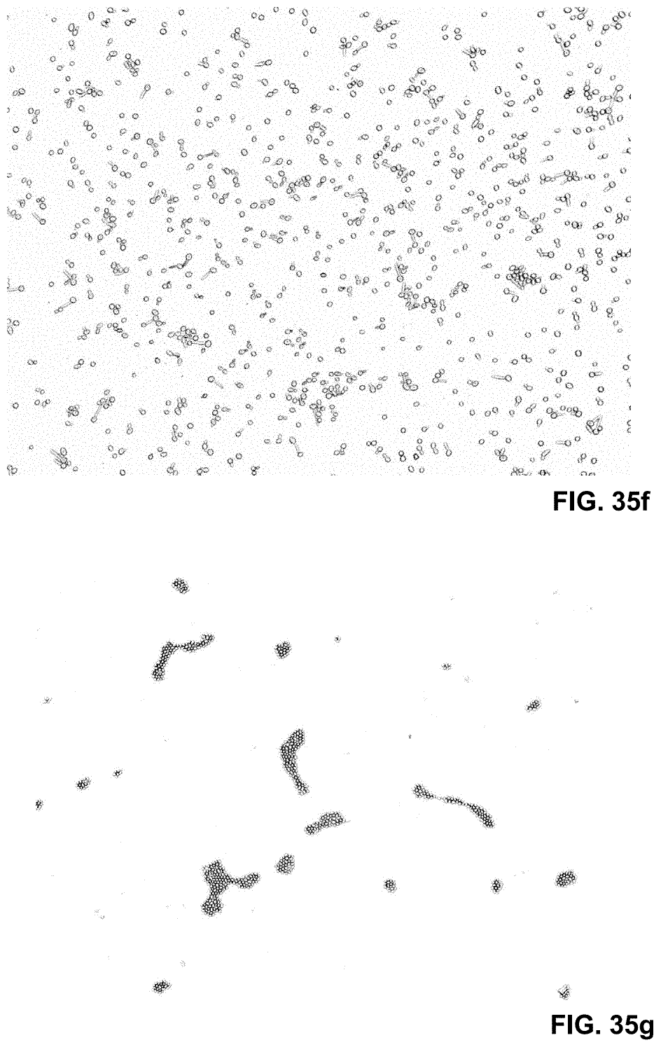

In FIGS. 35a to 35g the glass surface has been functionalized with a solution of 0.1 milligram per millilitre chitosan solution during 5 minutes at 25° C. in a first step. Thereafter, a dispersion comprising Candida albicans SC5314 and (a) 0.04% agarose by weight per total volume of the first solution (b) 0.04% agar by weight per total volume of the first solution (c) 0.25% alginate by volume per total volume of the first solution, (d) 0.25% Nafion® by weight per total volume of the first solution, (e) 0.4 cst polydimethylsiloxane, and (f) 0.125% polyethylene glycol d by weight per total volume of the first solution were added to the functionalized surface, respectively. In (g) the attachment of a suspension of Candida albicans SC5314 in the absence of a first compound is depicted. As follows from these figures the presence of chitosan alone resulted in few cells attached and regrouped in agglomerates. Usage of a first compound in the cell dispersion increased the number of cells attached and the homogeneity of the attachment.

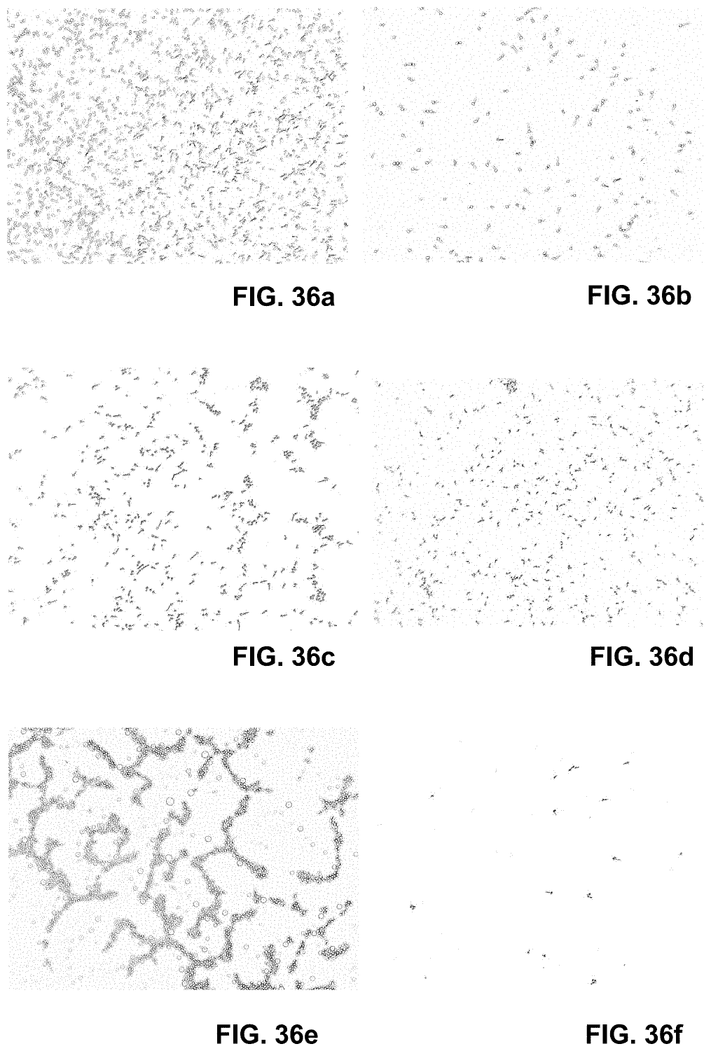

FIGS. 36a to 36f depict a glass surface that has been functionalized with a solution of 0.5% glutaraldehyde by volume per total volume of said solution in a first step. Thereafter, a dispersion comprising Candida albicans SC5314 and (a) 0.04% agarose by weight per total volume of the first solution (b) 0.04% agar by weight per total volume of the first solution (c) 0.25% alginate by volume per total volume of the first solution, (d) 0.25% Nafion® by weight per total volume of the first solution (e) 0.4 cst polydimethylsiloxane, and (f) 0.125% polyethylene glycol by weight per total volume of the first solution were added to the functionalized surface, respectively. As follows from these figures the addition of a first compound to the cell suspension results in a homogeneous repartition of the cells.

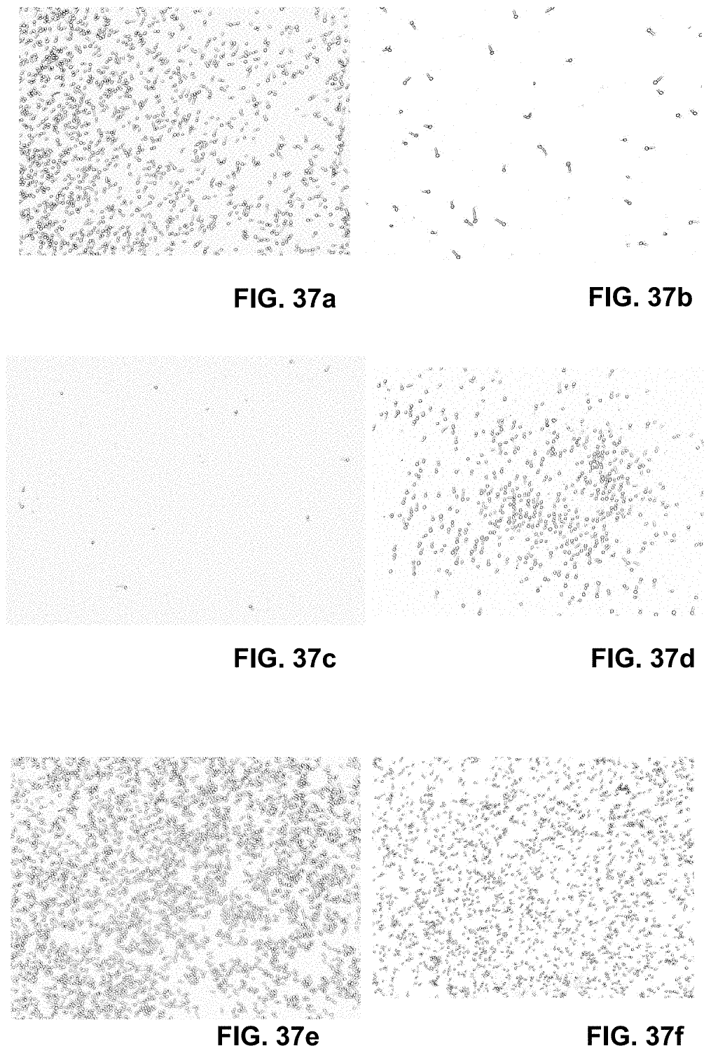

In FIGS. 37a to 37f the glass surface has been functionalized with a solution of (3-aminopropyl)triethoxysilane of 1% by volume per total volume of said solution during 5 minutes at 25° C. in a first step. Thereafter, a dispersion comprising Candida albicans SC5314 and (a) 0.04% agarose by weight per total volume of the first solution (b) 0.04% agar by weight per total volume of the first solution, (c) 0.25% alginate by volume per total volume of the first solution, (d) 0.25% Nafion® by weight per total volume of the first solution, (e) 0.4 cst polydimethylsiloxane, and (f) 0.125% polyethylene glycol by weight per total volume of the first solution were added to the functionalized surface, respectively. The addition of these first compounds to the cell suspension resulted in an absence of any agglomerates and in a homogeneous cell dispersion instead.

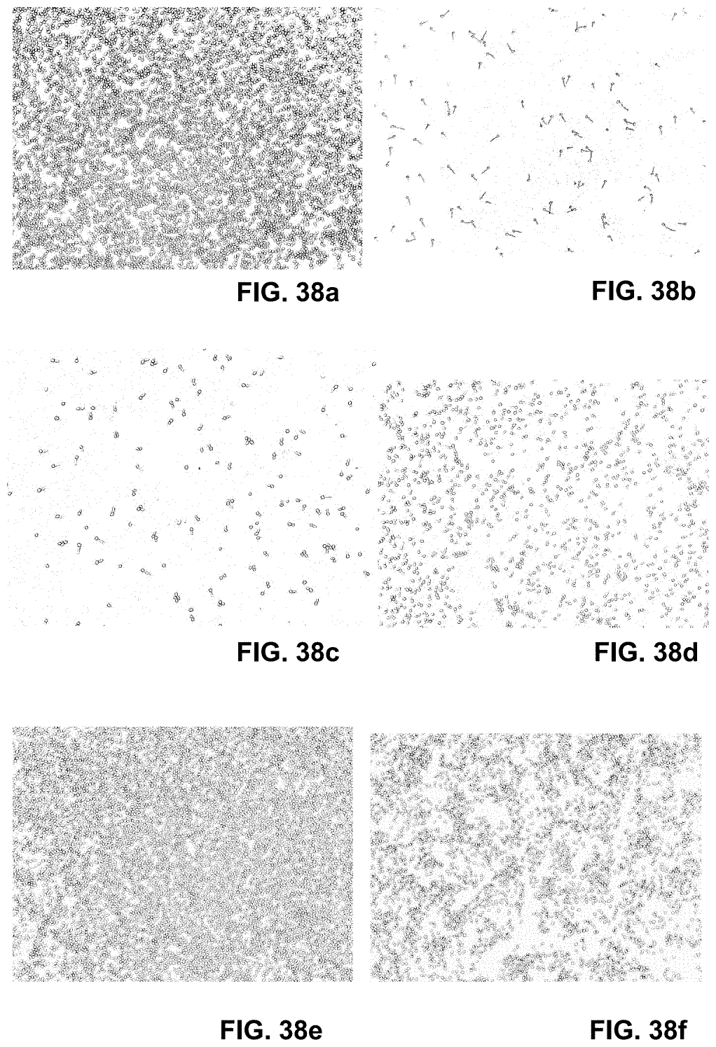

In FIGS. 38a to 38f the glass surface has been functionalized with a solution comprising 1 milligram per millilitre of poly(sodium-p-styrene sulfonate) and 1 milligram per millilitre of poly(allylamine hydrochloride copolymer during 20 minutes at 25° C. in a first step as described above with reference to FIGS. 27a to 27f. Thereafter, a dispersion comprising Candida albicans SC5314 and (a) 0.04% agarose by weight per total volume of the first solution (b) 0.04% agar by weight per total volume of the first solution, (c) 0.25% alginate by volume per total volume of the first solution (d) 0.25% Nafion® by weight per total volume of the first solution, (e) 0.4 cst polydimethylsiloxane, and (f) 0.125% polyethylene glycol by weight per total volume of the first solution were added to the functionalized surface, respectively. As is readily apparent from these figures, the cell dispersion according to the invention enabled a homogeneous attachment of many cells.

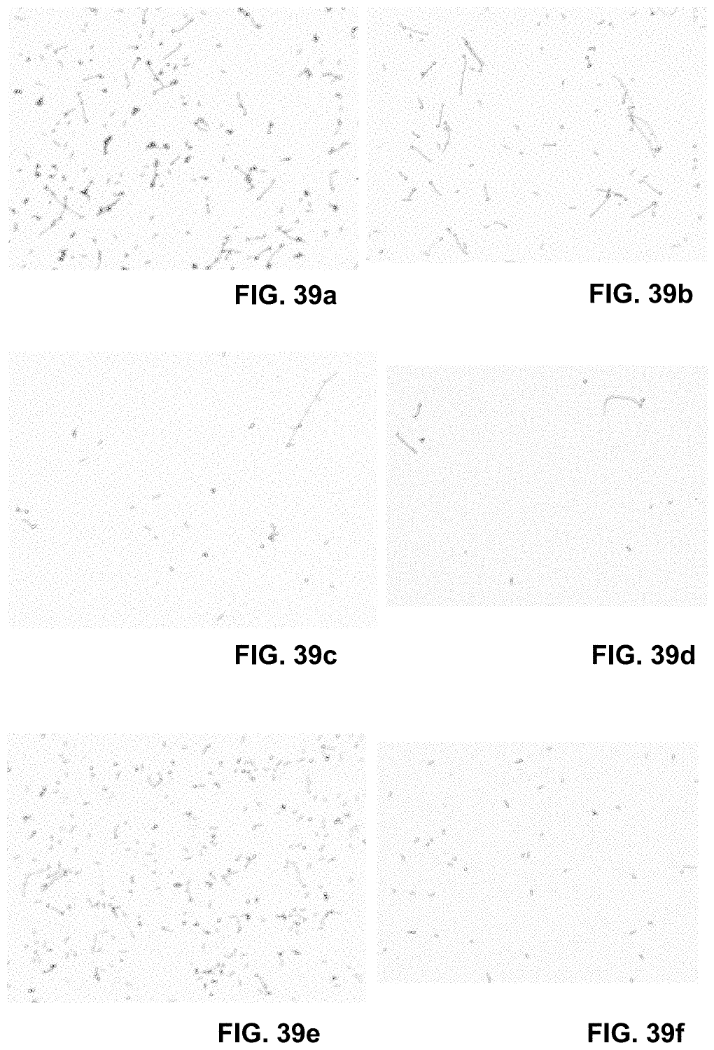

In FIGS. 39a to 39f the glass surface has been functionalized with a solution of 1 milligram per millilitre Mussel Adhesive recombinant protein (MAPTrix™) solution during 30 minutes at 25° C. in a first step. Thereafter, a dispersion comprising Candida albicans SC5314 and (a) 0.04% agarose by weight per total volume of the first solution (b) 0.04% agar by weight per total volume of the first solution, (c) 0.25% alginate by volume per total volume of the first solution, (d) 0.25% Nafion® by weight per total volume of the first solution, (e) 0.4 cst polydimethylsiloxane, and (f) 0.125% polyethylene glycol by weight per total volume of the first solution were added to the functionalized surface, respectively. Also in this case it is noted that a homogeneous attachment was achieved, wherein a rather high number of attachment was achieved especially with agarose and PDMS as first compound.

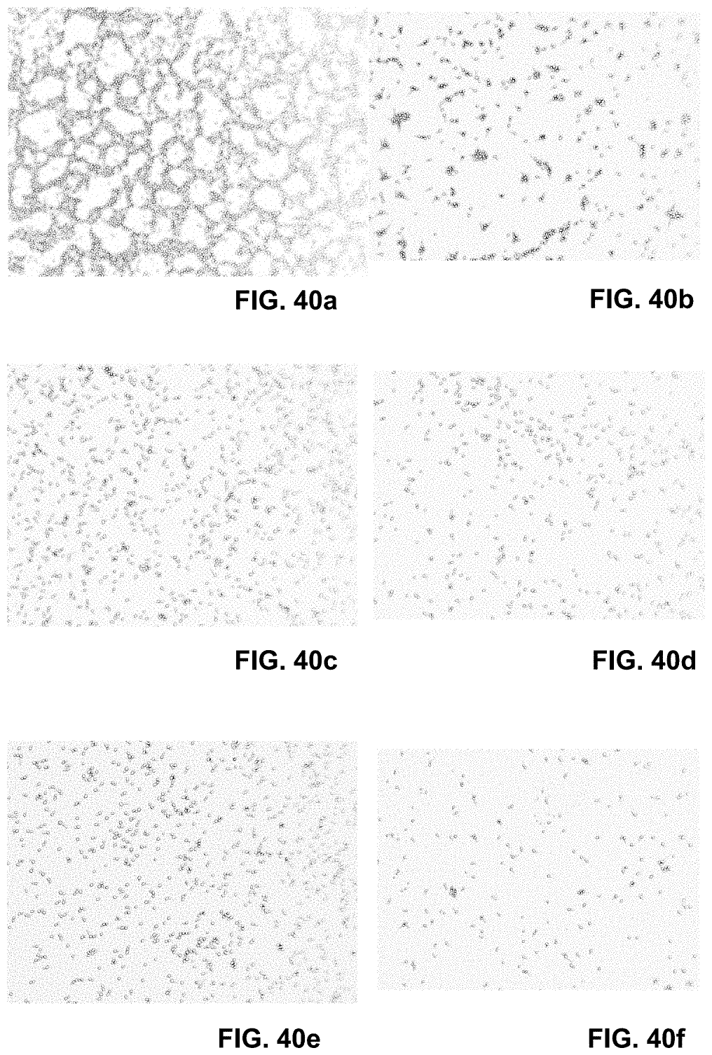

In FIGS. 40a to 40f the glass surface has been functionalized with a 10 mM solution of 4-aminothiophenol during 20 minutes at 25° C. in a first step. Thereafter, a dispersion comprising Candida albicans SC5314 and (a) 0.04% agarose by weight per total volume of the first solution (b) 0.04% agar by weight per total volume of the first solution, (c) 0.25% alginate by volume per total volume of the first solution, (d) 0.25% Nafion® by weight per total volume of the first solution, (e) 0.4 cst polydimethylsiloxane, and (f) 0.125% polyethylene glycol by weight per total volume of the first solution were added to the functionalized surface, respectively. The addition of the first compound to the cell suspension increased the homogeneity of the attachment. Especially Nafion® and alginate as first compounds gave homogeneous cell attachment with almost no agglomerates.

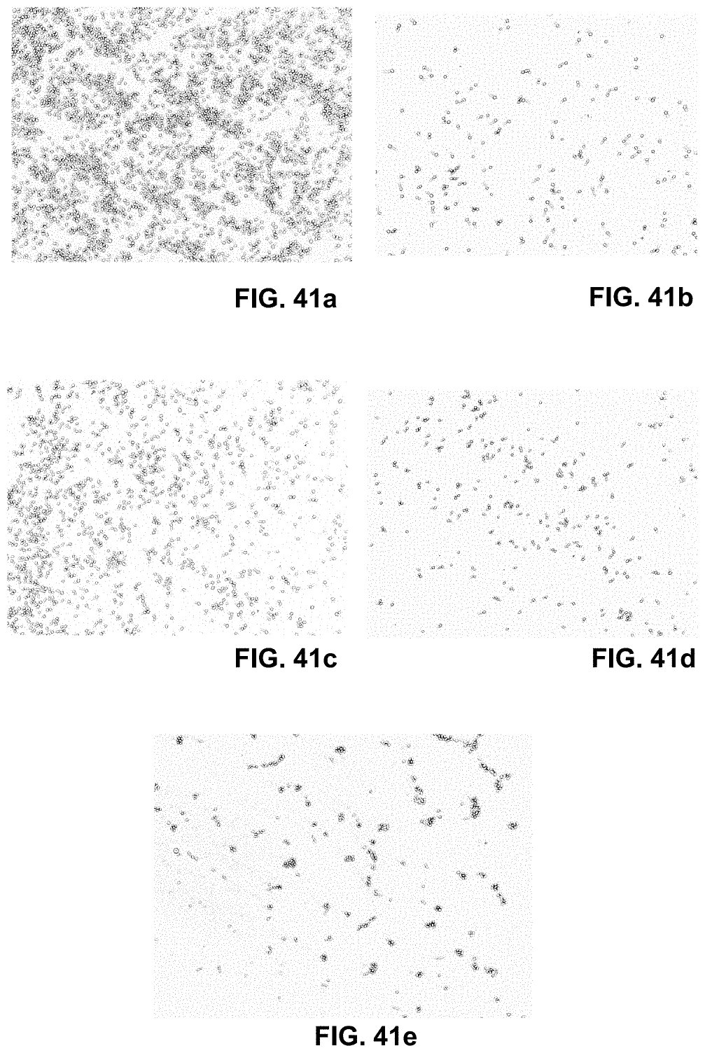

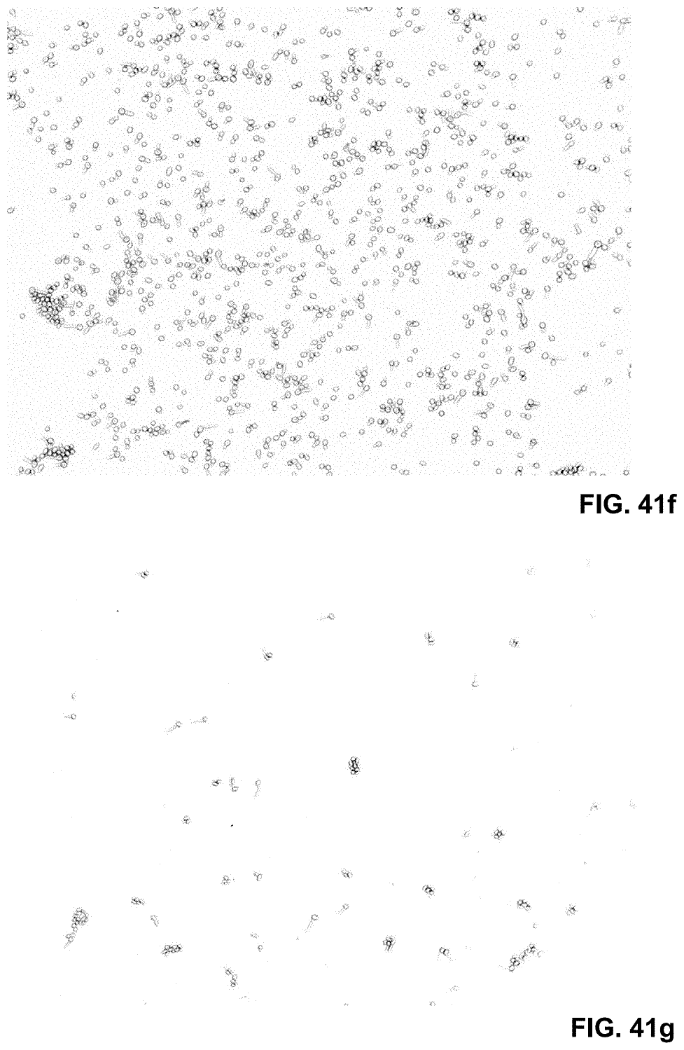

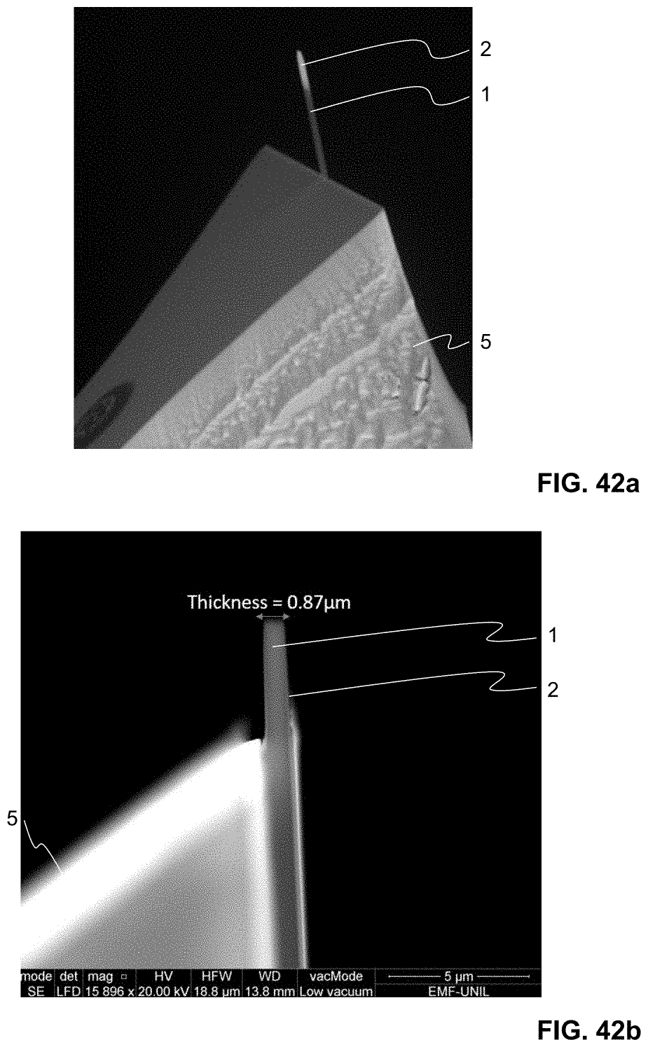

In FIGS. 41a to 41f the cell dispersion comprising Candida albicans SC5314 and a first compound was added to the unfunctionalized substrate. The first compounds were as follows: (a) 0.04% agarose by weight per total volume of the first solution (b) 0.04% agar by weight per total volume of the first solution, (c) 0.25% alginate by volume per total volume of the first solution, (d) 0.25% Nafion® by weight per total volume of the first solution, (e) 0.4 cst polydimethylsiloxane, and (f) 0.125% polyethylene glycol by weight per total volume of the first solution. FIG. 41g depicts an image of the untreated substrate to which the said cell suspension in the absence of any first compound has been added. The addition of a first compound resulted in increased attachment quality. Especially the addition of agarose or PEG resulted in a high amount of cells being attached.

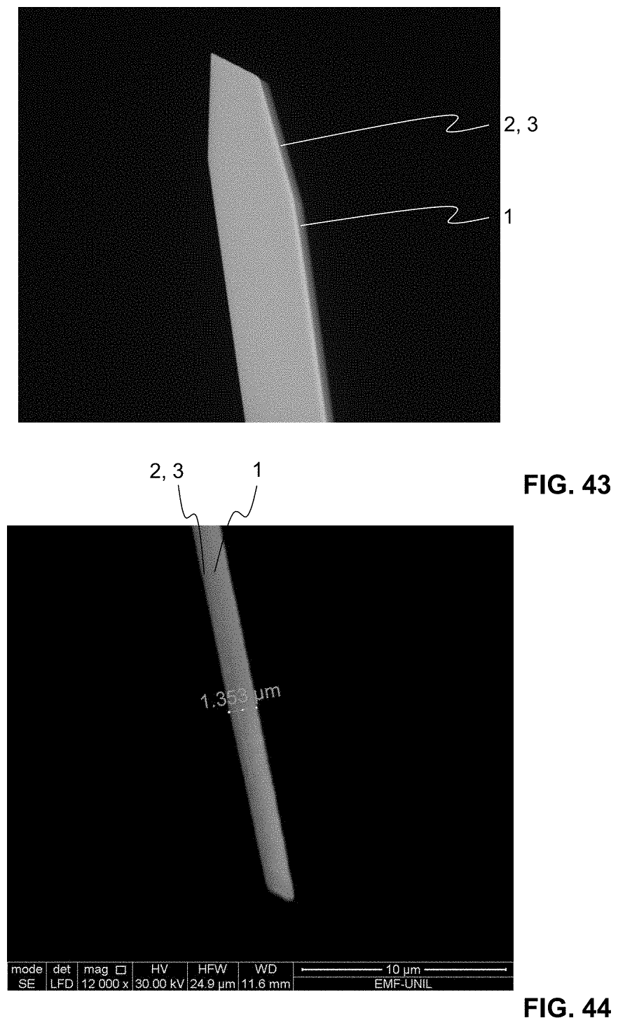

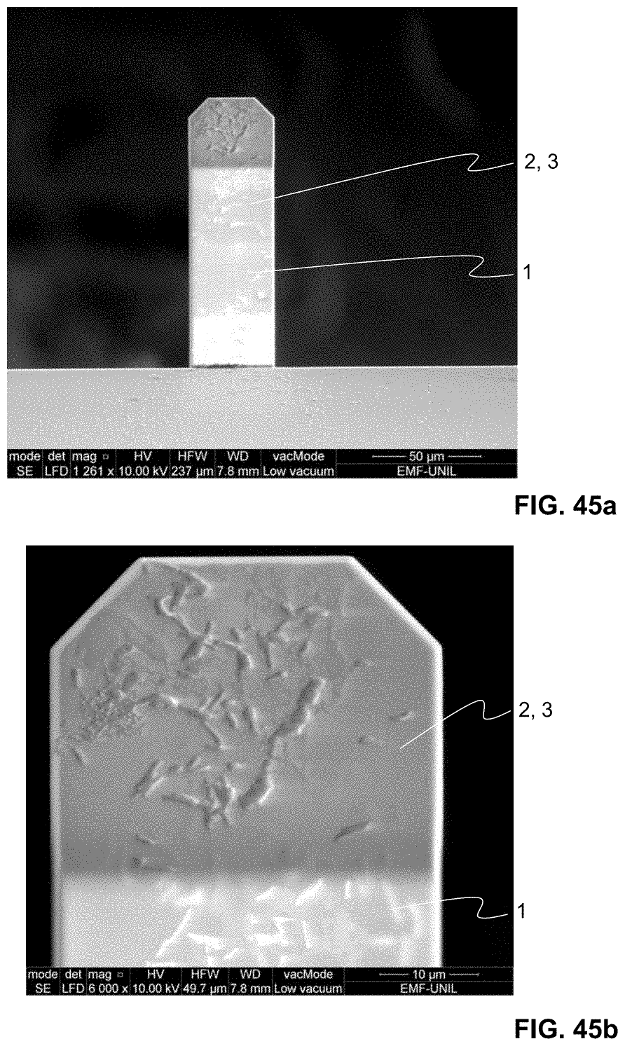

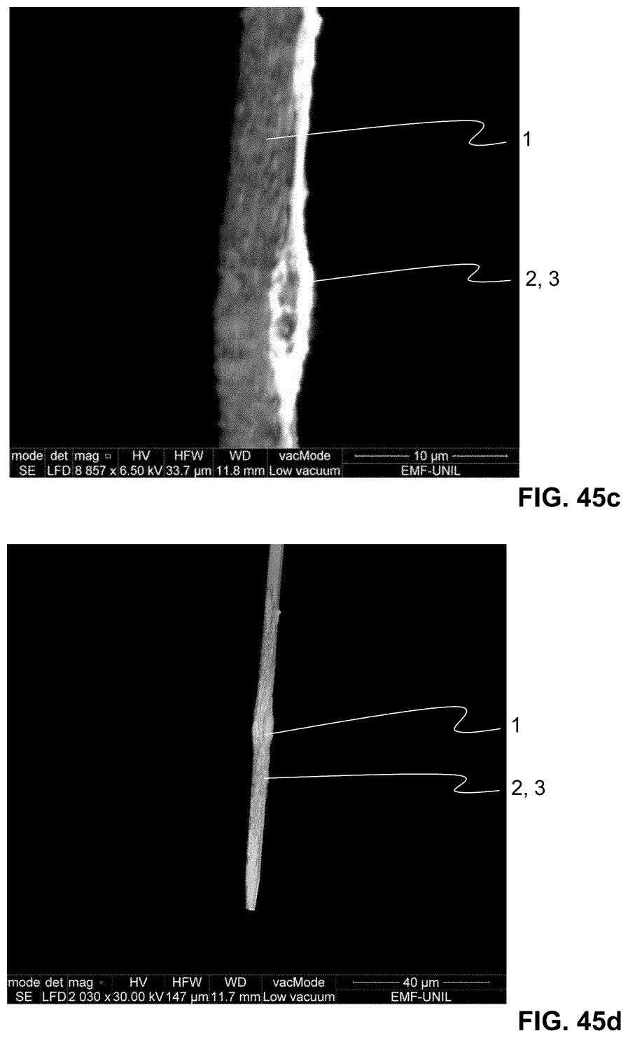

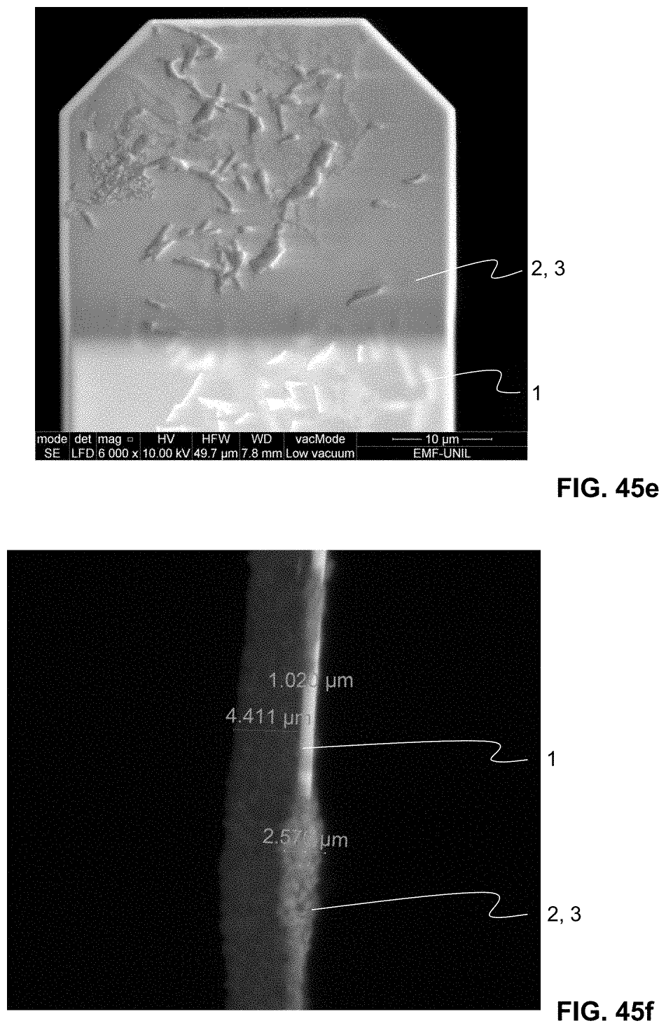

FIGS. 42a to 45f depict images of a substrate in the form of a cantilever being attached to a mount that were recorded with an electron microscope. To this end FIGS. 42a and 42b depict an untreated cantilever 1 whose surface 2 has not been functionalized yet. FIG. 43 depicts the cantilever 1 whose surface 2, 3 has been functionalized with a solution comprising 0.1 milligram per millilitre of poly-D-lysine solution for 20 minutes at 25° C. FIG. 44 depict the cantilever 1 according to FIG. 43 wherein the functionalized surface 3 has been additionally treated with a solution of 0.04% agarose by weight per total volume of said solution, incubated during 5 minutes at 25° C. FIGS. 45a to 45f depict images of the cantilever according to FIG. 43, wherein the functionalized surface has been additionally treated with a dispersion comprising E. coli ATCC 25922 submerged in an agarose solution. As just described, the functionalization of the cantilever was achieved by adding a solution comprising 0.1 milligram per millilitre of poly-D-lysine for 20 minutes at 25° C. in a first step and by incubating said functionalized cantilever with a dispersion comprising 0.04% weight by volume of agarose and Escherichia coli ATCC 25922 with an OD600=5 during 5 minutes at 25° C. in a second step. From these images it follows that the poly-D-lysine solution that is used to functionalize the surface of the cantilever forms a coating or a layer on the surface. It is said coating or layer that provides the functionalization of the cantilever. The addition of the agarose solution only (FIG. 44) as well as the addition of a cell dispersion comprising the bacteria as well as agarose (FIGS. 45a to 45f) in each case results in the formation of a further layer that is arranged on top of the layer constituting the functionalization of the cantilever. In other words, the functionalized cantilever to which the cells are attached can be seen as a layered device, wherein a first layer is arranged on top of a second layer. A thickness of the cantilever comprising the second layer only, i.e. a cantilever whose surface has been functionalized with a solution comprising the second compound according to the invention, has here a thickness in the range of about 720 to 780 nanometer. The thickness of the cantilever comprising the said second layer as well as a first layer being constituted by the agarose solution only has a thickness of about 1 micrometer to 1.5 micrometer. The thickness of a cantilever comprising the said second layer as well as a first layer being constituted by the dispersion comprising the bacteria being dispersed in the agarose solution is about 2.5 micrometer.

Regarding the attachment of other objects such as DNA the following is noted. DNA is negatively charged just as Gram-neg and Gram-pos bacteria. DNA backbone is constituted of phosphate groups which are negatively charged. In Gram-positive bacteria the reason of this negative charge is the presence of teichoic acids linked to either the peptidoglycan or to the underlying plasma membrane. These teichoic acids are negatively charged because of presence of phosphate in their structure. The Gram-negative bacteria have an outer covering of phospholipids and lipopolysaccharides. The lipopolysaccharides impart a strongly negative charge to surface of Gram-negative bacterial cells. The addition of a first compound according to the invention, i.e. the addition of a gelling agent and/or gellable agend and/or thickening agent, will help with the DNA distribution and the attachment on the substrate. Besides, it is noted that polysaccharides such as agar and agarose are commonly used in laboratories working with DNA already in these days, wherein agar and agarose are used to create hydrogels allowing DNA extraction and verification, for example.

### LIST OF REFERENCE SIGNS

1, 1′ substrate
2, 2′ surface
3, 3′, 3a′, 3b′, 3c′ functionalized surface
4a, 4b, 4c surface structure
5 mount
T transverse direction
L longitudinal direction

1. A kit-of-parts for attaching an object on a substrate comprising:
(i) At least a first solution comprising at least one first compound, wherein the first compound is at least one of a gelling agent, a gellable agent, and a thickening agent; and
(ii) At least a first substrate comprising a surface;
wherein the first solution is suitable for forming at least a first dispersion of at least one object in the first solution when at least one object is added to the first solution, and
wherein the first dispersion is suitable for attaching the object on the surface of the substrate when the first dispersion is added to the surface of the substrate.

2. The kit-of-parts according to claim 1, further comprising instructions for the attachment of the object, wherein the instructions comprise the step of preparing the first dispersion by dispersing the at least one object in the first solution, and
wherein the instructions further comprise the step of adding the first dispersion to the optionally functionalized surface of the substrate so as to attach the object on the optionally functionalized surface of the substrate.

3. The kit-of-parts according to claim 1, wherein the first solution comprises a pH-value that is at least one of physiological, in the range of about 6 to 8, or about 7.

4. The kit-of-parts according to claim 1, wherein at least one of i) at least a first part of the surface of the substrate is at least one of physically or chemically functionalized and ii) the kit-of-parts further comprises at least one second compound being suitable for chemically functionalizing at least a first part of the surface of the substrate, and
wherein the first dispersion and the functionalized surface of the substrate are suitable for attaching the object on the functionalized surface of the substrate when the first dispersion is added to the functionalized surface of the substrate.

5. The kit-of-parts according to claim 4, wherein at least one of i) the surface of the substrate is chemically functionalized, and wherein the at least one second compound is provided in at least one second solution, or
ii) wherein the surface of the substrate is physically functionalized, and wherein at least one of a) at least one surface structure is generated in the surface of the substrate or b) at least one layer is generated on the surface of the substrate.

6. The kit-of-parts according to claim 5, wherein the at least one layer comprises at least one of at least one metal compound, at least one oxide compound, at least one silicon compound, at least one nitride compound, at least one sulphide compound.

7. The kit-of-parts according to claim 5, wherein the second compound is at least one of a polymer or a copolymer thereof, a polymerizable agent, a cross-linking agent, and a compound comprising at least one functional group.

8. The kit-of-parts according to claim 7, wherein at least one of i) the polymer or the copolymer thereof or ii) the polymerizable agent is at least one of a polysaccharide compound, a polyaminosaccharide compound, a polyaminoacid compound, a polydopamine compound, a glycoproteine compound, a nucleic acid compound, an epoxy resin compound, a polysilane compound, a polysiloxane compound, a polyphosphate compound, a boron nitride polymer compound, a fluoropolymer compound, a polyallylamine compound, a polysulfide compound, and a polyphenol compound.

9. The kit-of-parts according to claim 8, wherein at least one of:
the polyaminosaccharide compound is chitosan,
the polyaminoacid compound is polylysine,
the glycoprotein compound is laminin,
the nucleic acid compound is desoxy ribonucleic acid,
the epoxy resin compound is at least one of a bisphenol polymer compound and polyacetylene compound,
the polyphenol compound is a polyphenolic protein, or
the polyallylamine compound comprises at least one of primary, secondary or tertiary polymers.

10. The kit-of-parts according to claim 7, wherein the cross-linking agent is an aldehyde-comprising cross-linking agent.

11. The kit-of-parts according to claim 7, wherein the functional group is at least one of an organosilicon compound or an organosulfur compound.

12. The kit-of-parts according to claim 5, wherein at least one of:
i) the second solution comprises at least one of a protic solvent, an aprotic solvent, a nonpolar solvent, a polar solvent, an organic compound, an inorganic compound, a liquid gas, and a melt, or
ii) the second solution is an aqueous solution that further comprises at least one of a polar water-soluble solvent such as an alcohol, a dissolved salt such as sodium chloride, and an acid such as acetic acid or hydrochloric acid.

13. The kit-of-parts according to claim 1, wherein the first compound is at least one of a polysaccharide, an amide-based polymer, a silicon-based polymer, and an ionomer.

14. The kit-of-parts according to claim 13, wherein at least one of:
the polysaccharide is selected from agarose, agar, alginate, dextran,
the amide-based polymer corresponds to polyacrylamide,
the silicon-based polymer corresponds to a polymeric organosilicon compound, or
the ionomer corresponds to an inorganic polymer.

15. The kit-of-parts according to claim 1, wherein at least one of:
i) the first solution is an aqueous solution, or
ii) the first solution is an aqueous solution that further comprises at least one of a growth medium or a preferably dissolved salt such as sodium chloride.

16. The kit-of-parts according to claim 1, wherein at least one of:
i) the first compound of the first solution has a concentration in the range of between 0.0001% by weight to 10% by weight with respect to a total volume of the first solution or between 0.001% by weight to 5% by weight with respect to the total volume of the first solution or between 0.02% by weight to 1% by weight with respect to the total volume of the first solution,
ii) wherein the first compound of the first solution has a concentration in the range of between 0.0001% by volume to 10% by volume with respect to a total volume of the first solution or between 0.001% by volume to 5% by volume with respect to the total volume of the first solution or between 0.02% by volume to 1% by volume with respect to the total volume of the first solution, or
iii) the first compound of the first solution is added to the first solution at a temperature between −20° C. to 120° C. or between 0° C. to 100° C. or between 10° C. and 40° C.

17. The kit-of-parts according to claim 5, wherein at least one of:
i) the second compound of the second solution has a concentration in the range of between 0.0001% by weight to 50% by weight with respect to a total volume of the second solution or between 0.001% by weight to 5% by weight with respect to the total volume of the second solution or between 0.01% by weight to 2% by weight with respect to the total volume of the second solution,
ii) wherein the second compound of the second solution has a concentration in the range of between 0.0001% by volume to 50% by volume with respect to a total volume of the second solution or between 0.001% by volume to 5% by volume with respect to the total volume of the second solution or between 0.01% by volume to 2% by volume with respect to the total volume of the second solution, or
iii) the second compound of the second solution is added to the second solution at a temperature between −100° C. to 500° C. or between 0° C. and 100° C. or between 10° C. and 40° C.

18. The kit-of-parts according to claim 1, wherein at least a second part of the surface of the substrate is functionalized, and
wherein at least one of:
i) the kit-of-parts comprises at least a further first solution comprising at least one further first compound that differs from the first compound of the first solution, or
ii) the functionalization of said second part of the surface of the substrate differs from the functionalization of the first part of the surface of the substrate.

19. The kit-of-parts according to claim 1, wherein at least one of:
i) the substrate is a flexible support or a rigid support;
ii) the substrate comprises a silicone-compound such as silicone dioxide or elementary silicone, plastic, ceramic, ceramic-metallic blend, a metal, a metal oxide or sulphide, and carbon such as graphite or diamond; or
iii) at least part of the surface of the substrate is coated with a coating prior to the functionalization of the surface of the substrate.

20. A method of producing a kit-of-parts for attaching an object on a substrate, the method comprising the steps of:
(i) Providing at least a first solution comprising at least one first compound, wherein the first compound is at least one of a gelling agent, a gellable agent, and a thickening agent; and
(ii) Providing at least a first substrate comprising a surface;
wherein the first solution is suitable for forming at least a first dispersion of at least one object in the first solution when at least one object is added to the first solution, and
wherein the first dispersion is suitable for attaching the object on the surface of the substrate when the first dispersion is added to the surface of the substrate.

21. The method according to claim 20, wherein at least one of i) at least a first part of the surface of the substrate is at least one of physically or chemically functionalized and ii) the method further comprises the step of providing at least one second compound being suitable for chemically functionalizing at least a first part of the surface of the substrate, and
wherein the first dispersion and the functionalized surface of the substrate are suitable for attaching the object on the functionalized surface of the substrate when the first dispersion is added to the functionalized surface of the substrate.

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. A method of attaching an object on a surface of a substrate comprising the steps of:
(i) Preparing at least a first dispersion of at least one object in a first solution, wherein the first solution comprises at least one first compound, wherein the first compound is at least one of a gelling agent, a gellable agent, and a thickening agent; and
(ii) Adding the first dispersion to the surface of the substrate, whereby the object is attached on the surface of the substrate.

35. The method according to claim 34, further comprising the step of functionalizing at least a first part of the surface of the substrate, and wherein the first dispersion is attached to the functionalized surface of the substrate, whereby the object is attached on the functionalized surface of the substrate.

36. The method according to claim 35, wherein at least one of:
i) the functionalization of the surface of the substrate corresponds to a chemical functionalization that is achieved by applying at least one second compound to the surface of the substrate, wherein the second compound is provided in at least one second solution and interacts with the surface of the substrate, or
ii) the functionalization of the surface of the substrate corresponds to a physical functionalization that is achieved by at least one of a) generating at least one surface structure in the surface of the substrate or by b) generating at least one layer on the surface of the substrate.

37. The method according to claim 36, wherein the at least one layer comprises at least one of: at least one metal compound, at least one oxide compound, at least one silicon compound, at least one nitride compound, or at least one sulphide compound.

38. (canceled)

39. (canceled)

40. The method according to claim 34, wherein at least a second part of the surface of the substrate is functionalized, and
wherein at least one of:
i) a further first dispersion of an object in a further first solution is added to the functionalized second part of the surface, wherein the further first solution comprises at least one further first compound that differs from the first compound of the first solution that is added to the functionalized first part of the surface, or
ii) the functionalization of said second part of the surface of the substrate differs from the functionalization of the first part of the surface of the substrate.

41. The method according to claim 34, wherein the object is a biological object being at least one of a cell, a virus such as a phage, and a matter of biological origin such as peptides, proteins, polysaccharides, vesicles, protein-RNA co-polymers, protein-DNA co-polymers, capsules, spores, or
wherein the object is a non-biological object being at least one of a protein, a lipid, a nucleic acid such as DNA, a nanotube or nano bead, a nanodevice, a glucide, a hydrocarbon, an aliphatic or aromatic polymer such as a phenolic polymer, and the like.

42. The method according to claim 34, wherein at least one of:
i) the substrate is a flexible support or a rigid support such as a glass cover slide, a ceramic tile, a rigid electrode, a dish;
ii) the substrate comprises a silicone-compound such as silicone dioxide or elementary silicone, plastic, ceramic, ceramic-metallic blend, a metal, a metal oxide or sulphide, and carbon such as graphite or diamond; or
iii) at least part of the surface of the substrate is coated with a coating prior to the functionalization of the surface of the substrate in step (ii).

43. (canceled)

44. A substrate comprising at least one object being attached thereon, and wherein the substrate comprises:
at least one surface, and
at least one first layer,
wherein the first layer is arranged on at least a part of the surface and is formed from at least a first dispersion as obtained in the method according to claim 34.

45. (canceled)

46. The substrate according to claim 44, wherein at least one of:
i) a thickness of the first layer is about 100 nanometer or more or about 1000 nanometer or more, or
ii) a thickness of the second layer is about 10 nanometer or more, or about 100 nanometer or more.
